SEQUENCE LISTING (113 sequences)

SEQ ID NO: 1 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
DLGVFRX

SEQ ID NO: 2 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
RCVFSGX

SEQ ID NO: 3 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
HVVGVIX

SEQ ID NO: 4 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
HVHSYLX

SEQ ID NO: 5 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
LNYFHGX

SEQ ID NO: 6 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
YILSIGX

SEQ ID NO: 7 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
CGLYNIX

SEQ ID NO: 8 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
CHSFFRX

SEQ ID NO: 9 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
GIRSLGX

SEQ ID NO: 10 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
ISCHYGX

SEQ ID NO: 11 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
IYFHHHX

SEQ ID NO: 12 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
VSYILLX

SEQ ID NO: 13 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
FNLVVDX

SEQ ID NO: 14 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
FFRGSDX

SEQ ID NO: 15 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
NRLDVSX

SEQ ID NO: 16 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
GLGHGNX

SEQ ID NO: 17 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
RVWQLCX

SEQ ID NO: 18 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
IVWQLCX

SEQ ID NO: 19 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
KVWQLAX

SEQ ID NO: 20 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
RVWCARX

SEQ ID NO: 21 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
RVYQVLX

SEQ ID NO: 22 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
QVWSAAX

SEQ ID NO: 23 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
RVSQVLX

SEQ ID NO: 24 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
KVWGGLX

SEQ ID NO: 25 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
RVYPVLX

SEQ ID NO: 26 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
QVWSARX

SEQ ID NO: 27 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
QVWCARX

SEQ ID NO: 28 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
TVWTCLX

SEQ ID NO: 29 - protein, 7 aa, synthetic construct
  VARIANT 7: note = Residue may be deleted
  VARIANT 6..7: note = Up to 29 "X" variables between position numbers 6 and 7 can be inserted
  REGION 1..7: note = Circular
KVYTAPX

SEQ ID NO: 30 - protein, 7 aa, synthetic construct
  REGION 1..7: note = Circular
CKVWQLL

SEQ ID NO: 31 - protein, 7 aa, synthetic construct
  REGION 1..7: note = Circular
CVQWLKL

SEQ ID NO: 32 - protein, 7 aa, synthetic construct
CKVWQLL

SEQ ID NO: 33 - protein, 7 aa, synthetic construct
  REGION 1..7: note = Circular
CEVWQLL

SEQ ID NO: 34 - protein, 7 aa, synthetic construct
  REGION 1..7: note = Circular
CKAWQLL

SEQ ID NO: 35 - protein, 7 aa, synthetic construct
  REGION 1..7: note = Circular
  REGION 1..7: note = Circular
CKVGQLL

SEQ ID NO: 36 - protein, 7 aa, synthetic construct
CEAWQLL

SEQ ID NO: 37 - protein, 7 aa, synthetic construct
  REGION 1..7: note = Circular
CEVGQLL

SEQ ID NO: 38 - protein, 7 aa, synthetic construct
  REGION 1..7: note = Circular
CKAGQLL

SEQ ID NO: 39 - protein, 7 aa, synthetic construct
  REGION 1..7: note = Circular
CEAGQLL

SEQ ID NO: 40 - protein, 7 aa, synthetic construct
  REGION 1..7: note = Circular
CRVWQLC

SEQ ID NO: 41 - protein, 7 aa, synthetic construct
  REGION 1..7: note = Circular
CIVWQLC

SEQ ID NO: 42 - protein, 7 aa, synthetic construct
  REGION 1..7: note = Circular
CKVWQLA

SEQ ID NO: 43 - protein, 7 aa, synthetic construct
  REGION 1..7: note = Circular
CRVWCAR

SEQ ID NO: 44 - protein, 7 aa, synthetic construct
  REGION 1..7: note = Circular
CRVYQVL

SEQ ID NO: 45 - protein, 7 aa, synthetic construct
  REGION 1..7: note = Circular
CQVWSAA

SEQ ID NO: 46 - protein, 7 aa, synthetic construct
  REGION 1..7: note = Circular
CRVSQVL

SEQ ID NO: 47 - protein, 7 aa, synthetic construct
  REGION 1..7: note = Circular
CKVWGGL

SEQ ID NO: 48 - protein, 7 aa, synthetic construct
  REGION 1..7: note = Circular
CRVYPVL

SEQ ID NO: 49 - protein, 7 aa, synthetic construct
  REGION 1..7: note = Circular
CQVWSAR

SEQ ID NO: 50 - protein, 7 aa, synthetic construct
  REGION 1..7: note = Circular
CQVWCAR

SEQ ID NO: 51 - protein, 7 aa, synthetic construct
  REGION 1..7: note = Circular
CTVWTCL

SEQ ID NO: 52 - protein, 7 aa, synthetic construct
  REGION 1..7: note = Circular
CKVYTAP

SEQ ID NO: 53 - protein, 7 aa, synthetic construct
  REGION 1..7: note = Circular
ARVWCAR

SEQ ID NO: 54 - protein, 7 aa, synthetic construct
  REGION 1..7: note = Circular
CAVWCAR

SEQ ID NO: 55 - protein, 7 aa, synthetic construct
  REGION 1..7: note = Circular
CRAWCAR

SEQ ID NO: 56 - protein, 7 aa, synthetic construct
  REGION 1..7: note = Circular
CRVACAR

SEQ ID NO: 57 - protein, 7 aa, synthetic construct
  REGION 1..7: note = Circular
CHVVGVI

SEQ ID NO: 58 - protein, 7 aa, synthetic construct
  REGION 1..7: note = Circular
CHVHSYL

SEQ ID NO: 59 - protein, 7 aa, synthetic construct
  REGION 1..7: note = Circular
CLNYFHG

SEQ ID NO: 60 - protein, 7 aa, synthetic construct
  REGION 1..7: note = Circular
CYILSIG

SEQ ID NO: 61 - protein, 7 aa, synthetic construct
  REGION 1..7: note = Circular
CCGLYNI

SEQ ID NO: 62 - protein, 7 aa, synthetic construct
  REGION 1..7: note = Circular
CCHSFFR

SEQ ID NO: 63 - protein, 7 aa, synthetic construct
  REGION 1..7: note = Circular
CGIRSLG

SEQ ID NO: 64 - protein, 7 aa, synthetic construct
  REGION 1..7: note = Circular
CISCHYG

SEQ ID NO: 65 - protein, 7 aa, synthetic construct
  REGION 1..7: note = Circular
CIYFHHH

SEQ ID NO: 66 - protein, 7 aa, synthetic construct
  REGION 1..7: note = Circular
CVSYILL

SEQ ID NO: 67 - protein, 7 aa, synthetic construct
CHVVGVI

SEQ ID NO: 68 - protein, 7 aa, synthetic construct
  REGION 1..7: note = Circular
CDLGVFR

SEQ ID NO: 69 - protein, 7 aa, synthetic construct
  REGION 1..7: note = Circular
CRCVFSG

SEQ ID NO: 70 - protein, 7 aa, synthetic construct
  REGION 1..7: note = Circular
CLNYFHG

SEQ ID NO: 71 - protein, 7 aa, synthetic construct
  REGION 1..7: note = Circular
CHVHSYL

SEQ ID NO: 72 - protein, 7 aa, synthetic construct
  REGION 1..7: note = Circular
CFNLVVD

SEQ ID NO: 73 - protein, 7 aa, synthetic construct
  REGION 1..7: note = Circular
CFFRGSD

SEQ ID NO: 74 - protein, 7 aa, synthetic construct
  REGION 1..7: note = Circular
CGIRSLG

SEQ ID NO: 75 - protein, 7 aa, synthetic construct
  REGION 1..7: note = Circular
CIYFHHH

SEQ ID NO: 76 - protein, 7 aa, synthetic construct
  REGION 1..7: note = Circular
CNRLDVS

SEQ ID NO: 77 - protein, 7 aa, synthetic construct
  REGION 1..7: note = Circular
CGLGHGN

SEQ ID NO: 78 - protein, 7 aa, synthetic construct
CDLGVFR

SEQ ID NO: 79 - protein, 7 aa, synthetic construct
CRCVFSG

SEQ ID NO: 80 - protein, 7 aa, synthetic construct
  REGION 1..7: note = Circular
AHVVGVI

SEQ ID NO: 81 - protein, 7 aa, synthetic construct
  REGION 1..7: note = Circular
CAVVGVI

SEQ ID NO: 82 - protein, 7 aa, synthetic construct
  REGION 1..7: note = Circular
CHAVGVI

SEQ ID NO: 83 - protein, 7 aa, synthetic construct
  REGION 1..7: note = Circular
CHVAGVI

SEQ ID NO: 84 - protein, 7 aa, synthetic construct
  REGION 1..7: note = Circular
CHVVAVI

SEQ ID NO: 85 - protein, 7 aa, synthetic construct
  REGION 1..7: note = Circular
CHVVGAI

SEQ ID NO: 86 - protein, 7 aa, synthetic construct
  REGION 1..7: note = Circular
CHVVGVA

SEQ ID NO: 87 - protein, 7 aa, synthetic construct
  REGION 1..7: note = Circular
CLDCVFG

SEQ ID NO: 88 - protein, 7 aa, synthetic construct
  REGION 1..7: note = Circular
CHLDIGC

SEQ ID NO: 89 - protein, 7 aa, synthetic construct
  REGION 1..7: note = Circular
CLSRRCG

SEQ ID NO: 90 - DNA, 18 bp, synthetic construct
  source 1..18: mol_type = other DNA
acagttgtgggcgatcgc

SEQ ID NO: 91 - DNA, 45 bp, synthetic construct
  source 1..45: mol_type = other DNA
acaactgtnnknnknnknnknnknnktgtctcagtttcggcaccg

SEQ ID NO: 92 - DNA, 45 bp, synthetic construct
  source 1..45: mol_type = other DNA
acaactgtndtndtndtndtndtndttgtctcagtttcggcaccg

SEQ ID NO: 93 - DNA, 26 bp, synthetic construct
  source 1..26: mol_type = other DNA
ggagtgcaaagaatatttgatattgg

SEQ ID NO: 94 - DNA, 18 bp, synthetic construct
  source 1..18: mol_type = other DNA
caatgggccgtactcaac

SEQ ID NO: 95 - DNA, 59 bp, synthetic construct
  source 1..59: mol_type = other DNA
acactctttccctacacgacgctcttccgatctggagtgcaaagaatatttgatattgg

SEQ ID NO: 96 - DNA, 52 bp, synthetic construct
  source 1..52: mol_type = other DNA
gtgactggagttcagacgtgtgctcttccgatctcaatgggccgtactcaac

SEQ ID NO: 97 - DNA, 20 bp, synthetic construct
  source 1..20: mol_type = other DNA
taatacgtctcactataggg

SEQ ID NO: 98 - DNA, 131 bp, synthetic construct
  source 1..131: mol_type = other DNA
aacaccacgtcgtccctatctgctgccctaggtctatgagtggttgctggataactttacgggcatgcataaggctcggtatctatattcagggagaccacaacggtttccctctacaaataattttgttt

SEQ ID NO: 99 - DNA, 131 bp, synthetic construct
  source 1..131: mol_type = other DNA
aacaccacgtcgtccctatctgctgccctaggtctatgagtggttgctggataactttacgggcatgcataaggctcgtaggatatattcagggagaccacaacggtttccctctacaaataattttgttt

SEQ ID NO: 100 - DNA, 172 bp, synthetic construct
  source 1..172: mol_type = other DNA
gatgaaattcgccacttgttagtgtaattcgctaactcatcctggcatgttgctgttgattcttcaatcagatctttataaatcaaaaagataaaaaattggcacgcaaattgtattaacagttcagcaggacaatcctgaacgcagaaatcaagaggacaacattacctgc

SEQ ID NO: 101 - DNA, 14 bp, synthetic construct
  source 1..14: mol_type = other DNA
aaaaaaggaaaaaa

SEQ ID NO: 102 - DNA, 15 bp, synthetic construct
  source 1..15: mol_type = other DNA
aaggaggaaaaaaaa

SEQ ID NO: 103 - DNA, 1275 bp, synthetic construct
  source 1..1275: mol_type = other DNA
atgaaaaaattattattcgcaattcctttagttgttcctttctattctcactccgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggtggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccttacgagttcagtatcgactgcgataagatcaacctgttccgcggtgtctttgcgtttcttttatatgttgccacctttatgtatgtattttctacgtttgctaacatactgcgtaataaggagtcttaa

SEQ ID NO: 104 - DNA, 1095 bp, synthetic construct
  source 1..1095: mol_type = other DNA
atgaaaaaattattattcgcaattcctttagttgttcctttctattctcactccgctgaaactgttcatcaccatcaccatcacgctgaaactgttgaaagttgtttagcaaaaccccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatccattcgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctcttcccaaatggctcaagtcggtgacggtgataattcacctttaatgaataatttccgtcaatatttaccttccctccctcaatcggttgaatgtcgcccttttgtctttggcgctggtaaaccttacgagttcagtatcgactgcgataagatcaacctgttccgcggtgtctttgcgtttcttttatatgttgccacctttatgtatgtattttctacgtttgctaacatactgcgtaataaggagtcttaa

SEQ ID NO: 105 - DNA, 2118 bp, synthetic construct
  source 1..2118: mol_type = other DNA
atgaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagagttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 106 - DNA, 2118 bp, synthetic construct
  source 1..2118: mol_type = other DNA
atgaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagagttcagtcatgacgctggcttacgggtccaaagagttcggcttccgtcaaagtgtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 107 - DNA, 681 bp, synthetic construct
  source 1..681: mol_type = other DNA
atggacgcagagttccgtcacgacagtggctatgaagtccaccaccagaaattggtttttttcgccgaggatgttggtagcaataaaggggctatcatcggtttaatggtaggtggagttgttattgcaggtggatctggtggtaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagtaa

SEQ ID NO: 108 - DNA, 681 bp, synthetic construct
  source 1..681: mol_type = other DNA
atggacgcagagttccgtcacgacagtggctatgaagtccaccaccagaaattggtttctttcgccgaggatgttggtagcaataaaggggctatcatcggtccgatggtaggtggagttgttattgcaggtggatctggtggtaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagtaa

SEQ ID NO: 109 - DNA, 666 bp, synthetic construct
  source 1..666: mol_type = other DNA
atgaaatgcaacactgccacatgtgcaacgcagcgcctggcaaattttttagttcattccagcaacaactttggtgccattctctcatctaccaacgtgggatccaatacatatggtggatctggtggtaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagtaa

SEQ ID NO: 110 - DNA, 657 bp, synthetic construct
  source 1..657: mol_type = other DNA
atggttaaagttatcggtcgtcgttccctcggagtgcaaagaatatttgatattggtcttccccaagaccataattttctgctagccaatggggcgatcgcccacaactgtaaggtgtggcagttgttgtgtctcagtttcggcaccgaaattttaaccgttgagtacggcccattgcccattggcaaaattgtgagtgaagaaattaattgttctgtgtacagtgttgatccagaagggagagtttacacccaggcgatcgcccaatggcatgaccggggagagcaggaagtattggaatatgaattggaagatggttcagtaatccgagctacctctgaccaccgctttttaaccaccgattatcaactgttggcgatcgaagaaatttttgctaggcaactggacttgttgactttagaaaatattaagcaaactgaagaagctcttgacaaccatcgtcttccctttccattacttgacgctgggacaattaaaacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaatga

SEQ ID NO: 111 - DNA, 658 bp, synthetic construct
  source 1..658: mol_type = other DNA
atggttaaagttatcggtcgtcgttccttcggagtgcaaagaatatttgatattggtcttccccaagaccataattttctgctagccaatggggcgatcgcccacaactgtaaggtgtggcagttgttgtgtctcagtttcggcaccgaaattttaaccgttgagtacggcccattgtccattggcaaaattgtgagtgaagaaattaattgttctgtgtacagtgttgatccaaaagggaaagtttacacccaggcgatcgcccaatggcatgaccggggagagcaggaagtattggaatatgaattggaagatggttcagtaatccgagctacctctgaccgccgctttttaaccaccgattatcgactggtggcgatcgaagaaatttttgcaaggcaactggacttgttgactttagaaaaaaattaagcaaactgaagaagctcttgacaaccatcgtcttccctttccattacttgacgctgggacaattaaaacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaatga

SEQ ID NO: 112 - DNA, 658 bp, synthetic construct
  source 1..658: mol_type = other DNA
atggttaaagttatcggtcgtcgttccctcggagtgcaaagaatatttgatattggtcttccccaagaccataattttctgctagccaatggggcgatcgcccacaactgtaaggtgtggcagttgttgtgtctcagtttcggcaccgaaattttaaccgttgagtacggcccattgcccattggcaaaattgtgagtgaagaaattaattgttctgtgtacagtgttgatccaaaagggagagtttacacccaggcgatcgcccaatggcatgaccggggagagcaggaagtattggaatatgaattggaagatggttcagtaatccgagctacctctgaccaccgctttttaaccaccgattatcaactgttggcgatcgaagaaatttttgctaggcaactggacttgttgactttagaaaaaaattaagcaaactgaagaagctcttgacaaccatcgtcttccctttccattacttgacgctgggacaattaaaacgacaaatcctggtgtatccgcttggcaggtcaacacagcttatactgcgggacaattggtcacatataacggcaagacgtataaatgtttgcagccccacacctccttggcaggatgggaaccatccaacgttcctgccttgtggcagcttcaatga

SEQ ID NO: 113 - DNA, 658 bp, synthetic construct; identical to SEQ ID NO: 111
  source 1..658: mol_type = other DNA

(19) United States
(12) Patent Application Publication
(10) Pub. No.: US 2025/0122242 A1
(43) Pub. Date: Apr. 17, 2025

(54) SELECTION SYSTEMS, PEPTIDES DETERMINED THEREWITH, AND METHODS OF USING SAME

(71) Applicant: Wisconsin Alumni Research Foundation, Madison, WI (US)

(72) Inventors: Tina Wang, Madison, WI (US); Linwei Yang, Madison, WI (US)

(73) Assignee: Wisconsin Alumni Research Foundation, Madison, WI (US)

(21) Appl. No.: 18/906,707

(22) Filed: Oct. 4, 2024

Related U.S. Application Data
(60) Provisional application No. 63/590,069, filed on Oct. 13, 2023.

(51) Int. Cl. C07K 7/64

(52) U.S. Cl. CPC C07K 7/64

(57) ABSTRACT

Selection systems, such as selection systems for determining peptides that inhibit protein aggregation, peptides determined with the selection systems, and methods of using the selection systems and the peptides.

### CROSS-REFERENCE TO RELATED APPLICATIONS

Priority is hereby claimed to U.S. Provisional Application 63/590,069, filed Oct. 13, 2023, which is incorporated herein by reference in its entirety.

### SEQUENCE LISTING

The instant application contains a Sequence Listing which has been submitted in XML format and is hereby incorporated by reference in its entirety. The XML copy, created on Sep. 27, 2024, is named USPTO—09824557-P230455US02—SEQ_LIST.xml and is 165,377 bytes in size.

### FIELD OF THE INVENTION

The invention is directed to selection systems, such as selection systems for determining peptides that inhibit protein aggregation, peptides determined with the selection systems, and methods of using the selection systems and the peptides.

### BACKGROUND

The aggregation of amyloidogenic proteins has been associated with numerous human diseases.¹ These proteins proceed through a series of misfolded intermediates to ultimately form the ordered, fibrillar structures known as amyloids, motivating the search for compounds that can disrupt their formation. However, structure-guided design of protein misfolding and aggregation inhibitors is complicated by the intrinsically disordered nature of many amyloidogenic species and the dearth of structural information on amyloid precursors.²³

Inhibitor discovery by high-throughput screening or selection often requires minimal structural information, providing a powerful alternative to rational design.⁴ A recent and exciting example is the screening of small molecule libraries for their effects on the kinetics of amyloid formation in vitro using purified protein.⁵ Such screens have identified promising leads,⁶ but nevertheless can be limited by throughput and the technical challenges of producing high-quality target protein to ensure reproducibility.⁵ Additionally, small molecules can be poorly suited for binding tightly to disordered misfolded species⁷ or strongly inhibiting the protein-protein interactions involved in misfolding and aggregation.⁸

Macrocyclic peptides have increasingly been recognized as a promising source of chemical probes and potential therapeutics.⁹⁻¹¹ These compounds can bind historically challenging targets, such as large or shallow protein surfaces, and enjoy improved stability and binding potency due to backbone rigidification.^{12,13} Platforms that select genetically encoded cyclic peptide libraries can search through an enormous number of sequences for ones that possess desired bioactivity. For example, mRNA and phage display methods have proven highly successful in identifying macrocyclic inhibitors for numerous targets.^{13,14} However, display techniques are typically constrained to identifying binders of immobilized proteins.¹³ A complementary approach, enabled by the rapidly emerging field of synthetic biology, involves the genetic or phenotypic selection of ribosomally synthesized cyclic peptide libraries in cells.¹⁵⁻¹⁸ Notably, this strategy can identify cyclic peptides with activities beyond single target binding,¹³ including inhibiting protein aggregation.¹⁹⁻²¹ Inhibitors of α-synuclein aggregation-induced toxicity have been identified by selecting cyclic peptides biosynthesized using the split-intein circular ligation of peptides and proteins (SICLOPPS) method¹⁵ in a yeast synucleinopathy model.¹⁹ Fluorescence-activated cell sorting has been used to select a SICLOPPS library for rescue of a GFP folding reporter in E. coli to identify cyclic peptide inhibitors of amyloid-β (Aβ) and mutant superoxide dismutase aggregation.^{20,21}

While powerful, these strategies for identifying cyclic peptide protein aggregation inhibitors are coupled to conventional selections such as cellular survival or fluorescence-based sorting, which are time- and labor-intensive and, in the case of cell sorting, require specialized equipment.²² Additionally, selections can suffer from false positives or uncover hits with undesirable properties,¹⁴ such as promiscuity or off-target activity. For example, the majority of hits selected in the Lindquist study were deemed to be false positives either arising from spontaneous mutations in the yeast strain or from off-target activity of the cyclic peptides themselves.¹⁹ Weeding out these false positive and off-target sequences then requires additional experiments, adding to time and labor.

Selection systems and methods capable of determining peptides, such as cyclic peptides, that inhibit aggregation of proteins are needed, as are peptides that inhibit aggregation.

### SUMMARY OF THE INVENTION

One aspect of the invention is directed to selection systems. In some versions, the selection systems comprise a host cell and a phage library comprising library phages configured to infect the host cell. In some versions, each library phage comprises a library gene configured to express a library peptide in the host cell. In some versions, at least a subset of the library peptides comprise different peptide sequences. In some versions, each library phage is deficient in a replication gene that encodes a replication protein. In some versions, the replication protein is a protein essential for production of infectious progeny phages from the host cell when infected with the library phages. In some versions, the selection systems further comprise a target fusion gene configured to express a target fusion protein in the host cell. In some versions, the target fusion protein comprises a target protein fused to a first subunit of a multi-subunit, promoter-specific RNA polymerase. In some versions, the selection systems further comprise one or more RNA-polymerase subunit genes configured to express one or more additional subunits of the RNA polymerase. In some versions, the selection systems further comprise a selection gene comprising a cognate promoter of the RNA polymerase operationally connected to a coding sequence of a selection protein. In some versions, the selection protein is the replication protein. In some versions, the selection protein is a dominant-negative form of the replication protein.

In some versions, the RNA polymerase and the cognate promoter are selected from the group consisting of a T7 RNA polymerase and a T7 promoter, a T3 RNA polymerase and a T3 promoter, and an SP6 RNA polymerase and an SP6 promoter, respectively.

In some versions, the library peptides comprise cyclic peptides. In some versions, at least one of the library peptides binds, is predicted to bind, or is suspected of binding to the target protein. In some versions, at least one of the library peptides binds to the target protein.

In some versions, the target protein is an aggregation-prone protein. In some versions, the target protein is an amyloidogenic protein.

In some versions, the replication gene with which the library phages are deficient and the selection protein encoded by the selection gene are selected from the group consisting of: gIII and either pIII or a dominant-negative form of pIII; gIV and either pIV or a dominant-negative form of pIV; gVI and either pVI or a dominant-negative form of pVI; gVII and either pVII or a dominant-negative form of pVII; gVIII and either pVIII or a dominant-negative form of pVIII; and gIX and either pIX or a dominant-negative form of pIX, respectively.

In some versions, the selection protein is the replication protein. In some versions, the selection protein is the dominant-negative form of the replication protein.

Another aspect of the invention is directed to methods of selection with a selection system of the invention. The methods can comprise a first selection. In some versions, the first selection comprises contacting a first population of host cells comprising multiple copies of the host cell with the phage library under conditions effective for the library phages to infect the host cells and thereby generate a first population of infected host cells, incubating the first population of infected host cells under conditions effective for production of first progeny phages to thereby produce a first selected phage library comprising the first progeny phages, and harvesting the first selected phage library.

The methods can further comprise a second selection. In some versions, the second selection comprises harvesting contacting a second population of host cells comprising multiple copies of the host cell with the first selected phage library under conditions effective for the first progeny phages to infect the host cells and thereby generate a second population of infected host cells, incubating the second population of infected host cells under conditions effective for production of second progeny phages to thereby produce a second selected phage library comprising the second progeny phages, and harvesting the second selected phage library.

In some versions, the first selection, the second selection, or both the first selection and the second selection are performed with a replication protein as a selection protein. In some versions, the first selection, the second selection, or both the first selection and the second selection are performed with a dominant-negative form of a replication protein as the selection protein. In some versions, the first selection is performed with a replication protein as a selection protein, and the second selection is performed with a dominant-negative form of a replication protein as the selection protein.

Another aspect of the invention is directed to cyclic peptides. In some versions, the cyclic peptides are head-to-tail cyclic peptides. In some versions, the cyclic peptides comprise an amino acid sequence selected from the group consisting of DLGVFRXₙ (SEQ ID NO:1), RCVFSGXₙ (SEQ ID NO:2), HWGVIXₙ (SEQ ID NO:3), HVHSYLXₙ (SEQ ID NO:4), LNYFHGXₙ (SEQ ID NO:5), YILSIGXₙ (SEQ ID NO:6), CGLYNIXₙ (SEQ ID NO:7), CHSFFRXₙ (SEQ ID NO:8), GIRSLGXₙ (SEQ ID NO:9), ISCHYGXₙ (SEQ ID NO:10), IYFHHHXₙ (SEQ ID NO:11), VSYILLXₙ (SEQ ID NO:12), FNLWDXₙ (SEQ ID NO:13), FFRGSDXₙ (SEQ ID NO:14), NRLDVSXₙ (SEQ ID NO:15), GLGHGNXₙ (SEQ ID NO:16), RVWQLCXₙ (SEQ ID NO:17), IVWQLCXₙ (SEQ ID NO:18), KVWQLAXₙ (SEQ ID NO:19), RVWCARXₙ (SEQ ID NO:20), RVYQVLXₙ (SEQ ID NO:21), QVWSAAXₙ (SEQ ID NO:22), RVSQVLXₙ (SEQ ID NO:23), KVWGGLXₙ (SEQ ID NO:24), RVYPVLXₙ (SEQ ID NO:25), QVWSARXₙ (SEQ ID NO:26), QVWCARXₙ (SEQ ID NO:27), TVWTCLXₙ (SEQ ID NO:28), and KVYTAPXₙ (SEQ ID NO:29) wherein X is any amino acid and n is an integer from 0-30.

Another aspect of the invention is directed to methods of reducing aggregation of an aggregation-prone protein. The methods can comprise contacting the aggregation-prone protein with a cyclic peptide of the invention. In some versions, the contacting is performed in vivo. In some versions, the contacting is performed in vitro.

In some versions, the aggregation-prone protein comprises human islet amyloid polypeptide. In some versions, the cyclic peptide has a sequence selected from the group consisting of DLGVFRXₙ (SEQ ID NO:1), RCVFSGXₙ (SEQ ID NO:2), HVVGVIXₙ (SEQ ID NO:3), HVHSYLXₙ (SEQ ID NO:4), LNYFHGXₙ (SEQ ID NO:5), YILSIGXₙ (SEQ ID NO:6), CGLYNIXₙ (SEQ ID NO:7), CHSFFRXₙ (SEQ ID NO:8), GIRSLGXₙ (SEQ ID NO:9), ISCHYGXₙ (SEQ ID NO:10), IYFHHHXₙ (SEQ ID NO:11), VSYILLXₙ (SEQ ID NO:12), FNLWDXₙ (SEQ ID NO:13), FFRGSDXₙ (SEQ ID NO:14), NRLDVSXₙ (SEQ ID NO:15), and GLGHGNXₙ (SEQ ID NO:16) wherein X is any amino acid and n is an integer from 0-30. In some versions the contacting is performed in a subject with type 2 diabetes.

In some versions, the aggregation-prone protein comprises amyloid-β42. In some versions, the cyclic peptide has a sequence selected from the group consisting of RVWQLCXₙ (SEQ ID NO:17), IVWQLCXₙ (SEQ ID NO:18), KVWQLAXₙ (SEQ ID NO:19), RVWCARXₙ (SEQ ID NO:20), RVYQVLXₙ (SEQ ID NO:21), QVWSAAXₙ (SEQ ID NO:22), RVSQVLXₙ (SEQ ID NO:23), KVWGGLXₙ (SEQ ID NO:24), RVYPVLXₙ (SEQ ID NO:25), QVWSARXₙ (SEQ ID NO:26), QVWCARXₙ (SEQ ID NO:27), TVWTCLXₙ (SEQ ID NO:28), and KVYTAPXₙ (SEQ ID NO:29) wherein X is any amino acid and n is an integer from 0-30. In some versions, the contacting is performed in a subject with Alzheimer's disease.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Strategy for cyclic peptide discovery. Selection phage (SP) carrying a library of genetically encoded cyclic peptides are used to infect host E. coli cells transformed with an “accessory plasmid.” The accessory plasmid encodes a selection that links a user defined inhibitory activity to expression of phage protein pill, which is required for phage reproduction. Upon infection, the cyclic peptide library member is expressed from the transduced SP genome and, if active, triggers production of pIII from the accessory plasmid. As a result, only SP that carry active library members propagate, allowing rapid enrichment of desired cyclic peptide sequences.

FIGS. 2A-2E. Design of a selection for cyclic peptide protein aggregation inhibitors. (FIG. 2A) Cartoon overview of a split T7 RNAP folding reporter strategy for identifying protein aggregation inhibitors. APP=aggregation-prone protein, T7n and T7c=N- and C-terminal halves of split-T7 RNAP, respectively. (FIG. 2B) Diagram of the genetic circuits that constitute the protein aggregation inhibitor selection. (FIG. 2C) Propagation activity of kanR-encoding phage on host cells encoding the APP-T7n selection, where the APP is either Aβ42 or its non-aggregating F19S/L34P mutant. (FIG. 2D) Comparison of phage propagation activities of SP carrying Aβ42 aggregation inhibitor cyclo-CKVWQLL (SEQ ID NO:30) or scrambled negative control cyclo-CVQWLKL (SEQ ID NO:31) on host cells encoding the Aβ42-T7n selection. (FIG. 2E) Effect of the mutation of key residues in cyclo-CKVWQLL (SEQ ID NO:30) on SP propagation on host cells encoding the Aβ42-T7n selection. (FIGS. 2C-2E) Data reflects mean and standard error (s.e.) of two (FIGS. 2C and 2D) or three (FIG. 2E) biological replicates. Fold phage propagation is calculated as the number of phage generated from an infected culture divided by the number of phage (10°) used to infect the culture. calculated as the number of phage generated from an infected culture divided by the number of phage generated from an infected culture divided by the number of phage (10°) used to infect the culture.

FIGS. 3A-3F. Selection of cyclic peptide Aβ42 aggregation inhibitors. (FIG. 3A) Graphical overview of selection procedure. (FIG. 3B) Phage titers (solid lines, plotted on left y-axis) and propagation activity (dashed lines, plotted on right y-axis) of successive rounds of selection of an SP-encoded cyclo-CX₃QLX library on selection cells encoding Aβ42 as the target APP. “In” refers to the input SP pool used to infect selection cells and “out” refers to the output SP pool obtained after selection, for a given round of selection. pfu=plaque-forming units. Fold phage propagation is calculated as the number of phage generated from an infected culture divided by the number of phage used to infect the culture. (FIG. 3C) Sanger sequencing of cyclic peptides sequences encoded by clonal phage isolated from round 7 of the selection. Residues in gray italics were fixed in the starting library. (FIG. 3D) Phage titers (solid lines, plotted on left y-axis) and propagation activity (dashed lines, plotted on right y-axis) of successive rounds of selection of an SP-encoded cycloCX6 library on selection cells encoding Aβ42 as the target APP. “In” refers to the input SP pool used to infect selection cells and “out” refers to the output SP pool obtained after selection, for a given round of selection. pfu=plaque-forming units. (FIG. 3E) Top 10 enriched cyclic peptide sequences after 7 rounds of selection on Aβ42 as determined by high-throughput sequencing analysis. (FIG. 3F) Propagation activity of clonal phage encoding enriched sequences CRVWCAR (SEQ ID NO:43) and CRVYQVL (SEQ ID NO:44) on host cells encoding the Aβ42-T7n selection. Phage encoding active sequence CKVWQLL (SEQ ID NO:30) is shown for comparison. Data reflect mean and s.e. of two biological replicates. (FIG. 3G) Propagation activity of clonal phage encoding single alanine substitutions of the CRVWCAR (SEQ ID NO:43) sequence on host cells encoding the Aβ42-T7n selection. Data reflect mean and s.e. of three biological replicates. (FIG. 3H) Evaluation of cyclic peptide sequences enriched by selection on Aβ42-T7n expressed from plasmid using the Aβ42-GFP folding reporter. CKVWQLL (SEQ ID NO:30) is shown for comparison. Cyclic peptides are induced at 0.04 mM IPTG. Data reflects mean and s.e. of three biological replicates. (FIGS. 3D, 3F, and 3G) Fold phage propagation is calculated as the number of phage generated from an infected culture divided by the number of phage used to infect the culture. For (FIGS. 3F and 3G), 105 input phage were used to infect.

FIGS. 4A-4J. Selection of cyclic peptide inhibitors of hIAPP aggregation. (FIG. 4A) Phage titer (solid line, plotted on left y-axis) and propagation activity (dashed line, plotted on right y-axis) of successive rounds of selection of an SP-encoded SICLOPPS library on selection cells targeting hIAPP aggregation. “In” refers to the input SP pool used to infect selection cells and “out” refers to the output SP pool obtained after selection, for a given round of selection. pfu=plaqueforming units. Fold phage propagation is calculated as the number of phage generated from an infected culture divided by the number of phage used to infect the culture. (FIG. 4B) Propagation activity of SP pools from each round of the selection shown in (FIG. 4A) where input phage titers are normalized to 105 pfu/mL. Data reflect two biological replicates plotted as individual values. (FIG. 4C) Top 10 enriched cyclic peptide sequences after 5 rounds of selection on hIAPP as determined by high-throughput sequencing analysis. (FIG. 4D) Structure of cyclo-CHVVGVI (SEQ ID NO:57). (FIG. 4E) Effect of varying concentrations of chemically synthesized cyclo-CHVVGVI (SEQ ID NO:57) (left) or linear CHVVGVI (SEQ ID NO:67) (right) on aggregation of 10 μM hIAPP measured by ThT fluorescence. Assay was run in phosphate-buffered saline (PBS) pH 6.9 at 32° C. under quiescent conditions. Data reflect plots of individual values of three technical replicates. The hIAPP alone (0 μM peptide) data is the same in both plots. (FIG. 4F) Effect of cyclo-CHVVGVI (SEQ ID NO:57) on lag time (tlag) of hIAPP aggregation. Data reflects tlag fits from four biological replicates (4 separate ThT assays; see FIG. 11) plotted as individual values. P-values (two-tailed Student's t-test between 0 μM peptide and each peptide concentration): *, P≤0.05; **, P≤0.01. (FIGS. 4G and 4H) TEM analysis of 10 μM hIAPP incubated in the presence (FIG. 4G) of 40 μM cyclo-CHVVGVI (SEQ ID NO:57) or alone (FIG. 4H) for for 2 h. (i-j) TEM analysis of 10 μM hIAPP in the presence (FIG. 4I) of 40 μM cyclo-CHVVGVI (SEQ ID NO:57) or alone (FIG. 4J) for 14 h. (FIGS. 4G-4J) For compatibility with TEM, hIAPP incubations were run in 25 mM sodium phosphate buffer, pH 7.4; see FIG. 13 for hIAPP aggregation kinetics under TEM assay conditions. Scale bars=200 nm.

FIGS. 5A-5E. Negative selection to remove promiscuous cyclic peptide sequences. (FIG. 5A) Propagation activity of the SP pool from round 5 of positive selection for hIAPP inhibition on selection cells encoding hIAPP, Aβ42, or an insoluble antibody fragment (scFv) fused to T7n. (FIG. 5B) Phage titer (solid line, plotted on the left y-axis) and propagation activity (dashed line, plotted on the right y-axis) of successive rounds of selection of the hIAPP round 5 SP pool on Aβ42-T7n negative selection cells. “In” refers to the input SP pool used to infect selection cells and “out” refers to the output SP pool obtained after selection, for a given round of selection. pfu=plaque-forming units. Fold phage propagation is calculated as the number of phage generated from an infected culture divided by the number of phage used to infect the culture. (FIG. 5C) Propagation activity of pre- and post-negative selection SP pools on selection cells encoding hIAPP or Aβ42 as the target APP. (FIG. 5D) Top 10 enriched cyclic peptide sequences after 4 rounds of negative selection as determined by high-throughput sequencing analysis. (FIG. 5E) Propagation of clonal phage encoding cyclic peptide sequences enriched by either positive selection on hIAPP only (CHVVGVI (SEQ ID NO:57)) or by hIAPP positive selection followed by negative selection against Aβ42 inhibitors (CDLGVFR (SEQ ID NO:68) and CRCVFSG (SEQ ID NO:69)) on selection cells encoding hIAPP or Aβ42 as the target APP. (FIGS. 5A, 5C, and 5E) Data reflects the mean and s.e. of two biological replicates. Propagations used an input of 105 phage.

FIGS. 6A-6J. Cyclic peptides identified by negative selection inhibit hIAPP aggregation. (FIGS. 6A and 6B) Structures of cyclo-CDLGVFR (SEQ ID NO:68) and cyclo-CRCVFSG (SEQ ID NO:69). (FIG. 6C) Effect of varying concentrations of chemically synthesized cyclo-CDLGVFR (SEQ ID NO:68) (left) or linear CDLGVFR (SEQ ID NO:78) (right) on aggregation of 10 μM hIAPP measured by ThT fluorescence. Assay was run in phosphate-buffered saline (PBS) pH 6.9 at 32° C. under quiescent conditions. Data reflect plots of individual values of three technical replicates. The hIAPP alone (0 μM peptide) data is the same in both plots. (FIG. 6D) Effect of cyclo-CDLGVFR (SEQ ID NO:68) on lag time (tlag) of hIAPP aggregation. Data reflects tlag fits from four biological replicates (4 separate ThT assays; see FIG. 18) plotted as individual values. P values (two-tailed Student's t-test between 0 μM peptide and each peptide concentration): **, P≤0.01. (FIG. 6E) Effect of varying concentrations of chemically synthesized cyclo-CRCVFSG (SEQ ID NO:69) (left) or linear CRCVFSG (SEQ ID NO:79) (right) on aggregation of 10 μM hIAPP measured by ThT fluorescence. Assay was run in phosphate-buffered saline (PBS) pH 6.9 at 32° C. under quiescent conditions. Data reflect plots of individual values of three technical replicates. The hIAPP alone (0 μM peptide) data is the same as in (FIG. 6C). (FIG. 6F) Effect of cyclo-CRCVFSG (SEQ ID NO:69) on lag time (tlag) of hIAPP aggregation. Data reflects tlag fits from four biological replicates (4 separate ThT assays; see FIG. 19) plotted as individual values. P-values (two-tailed Student's t-test between 0 μM peptide and each peptide concentration): **, P≤0.01. (FIGS. 6G and 6H) TEM analysis of 10 μM hIAPP incubated in the presence of 40 μM cyclo-CDLGVFR (SEQ ID NO:68) (FIG. 6G) or cyclo-CRCVFSG (SEQ ID NO:69) (FIG. 6H) for 2 h. (i-j) TEM analysis of 10 μM hIAPP incubated in the presence of 40 μM cyclo-CDLGVFR (SEQ ID NO:68) (FIG. 6G) or cyclo-CRCVFSG (SEQ ID NO:69) (FIG. 6H) for 14 h. (FIGS. 6G-6J) For compatibility with TEM, hIAPP incubations were run in 25 mM sodium phosphate buffer, pH 7.4; see FIG. 13 for hIAPP aggregation kinetics under TEM assay conditions. Scale bars=200 nm.

FIG. 7. Mechanism of head-to-tail cyclic peptide formation by SICLOPPS.

FIGS. 8A-8F. (FIG. 8A) Propagation of parent SP encoding CKVWQLL (SEQ ID NO:30) or scrambled control CVQWLKL (SEQ ID NO:31) on host cells carrying the Aβ42 selection. Data reflects mean and standard error (s.e.) of 2 biological replicates. Fold phage propagation is calculated as the number of phage generated from an infected culture divided by the number of phage (105) used to infect the culture. (FIG. 8B) Phage titers from phage-assisted continuous evolution (PACE) of SP encoding the SICLOPPS precursor for cyclo-CKVWQLL (SEQ ID NO:30) on selection cells encoding Aβ42-T7n using AP pTW006aP1a. pfu=plaque-forming units. (FIG. 8C) Mutations from clonal SP isolated from the last timepoint of PACE 1. (FIG. 8D) Evaluation of clonal SP activity on selection cells encoding Aβ42-T7n using AP pTW357b. Active (CKVWQLL ((SEQ ID NO:30))), scrambled control (CVQWLKL ((SEQ ID NO:31))), and intein splicing-inactivated (CKVWQLL* (SEQ ID NO:32)) cyclic peptide sequences were evaluated. Data reflects mean and standard error (s.e.) of 2 biological replicates. Fold phage propagation is calculated as the number of phage generated from an infected culture divided by the number of phage (10⁵) used to infect the culture. (FIG. 8E) Overview of the Aβ42-GFP assay for detecting Aβ42 aggregation inhibition. (FIG. 8F) Evaluation of either parent or evolved (clone 2) SICLOPPS cyclic peptide precursor expressed from plasmid using the Aβ42-GFP folding reporter. Active (CKVWQLL (SEQ ID NO:30)), negative control (CEAGQLL (SEQ ID NO:39)), and intein splicing-inactivated (CKVWQLL* (SEQ ID NO:32)) cyclic peptide sequences were evaluated. Cyclic peptides are induced at 0.04 mM IPTG. Data reflects mean and s.e. of 3 biological replicates.

FIGS. 9A and 9B. (FIG. 9A) Sequence logos generated from the top 50 enriched sequences from selection of a phage-encoded cyclo-CX₆ library for Aβ42-T7n inhibition. (FIG. 9B) OD₆₀₀ of cells expressing SICLOPPS precursors for cyclo-CKVWQLL (SEQ ID NO:30), cyclo-CRVWCAR (SEQ ID NO:43), and cyclo-CRVYQVL (SEQ ID NO:44) upon induction by IPTG. Data reflects 3 biological replicates plotted as individual values.

FIGS. 10A-10D. (FIG. 10A) Phage titers from PACE of SP encoding the SICLOPPS precursor for cyclo-CKVWQLL (SEQ ID NO:30) on selection cells encoding Aβ42-T7n using AP pTW357d5. pfu=plaque-forming units. (FIG. 10B) Mutations from clonal SP isolated from the last timepoint of PACE 2. (FIG. 10C) Evaluation of clonal SP activity on selection cells encoding Aβ42-T7n using AP pTW357d5. Active (CKVWQLL (SEQ ID NO:30)), negative control (CEAGQLL (SEQ ID NO:39)), and intein splicing-inactivated (CKVWQLL* (SEQ ID NO:32)) cyclic peptide sequences were evaluated. Data reflects mean and standard error (s.e.) of two biological replicates. Fold phage propagation is calculated as the number of phage generated from an infected culture divided by the number of phage (10⁵) used to infect the culture. (FIG. 10D) Evaluation of either parent or evolved (PACE 2 clone 1) SICLOPPS cyclic peptide precursor expressed from plasmid using the Aβ42-GFP folding reporter. Active (CKVWQLL (SEQ ID NO:30)), negative control (CEAGQLL (SEQ ID NO:39)), and intein splicing-inactivated (CKVWQLL* (SEQ ID NO:32)) cyclic peptide sequences were evaluated. Cyclic peptides are induced at 0.04 mM IPTG. Data reflects mean and s.e. of 3 biological replicates.

FIG. 11. Replicates of hIAPP aggregation assays with cyclo-CHVVGVI (SEQ ID NO:57) used to calculate t_{lag} values shown in FIG. 4F. Each graph reflects plots of individual values of three technical replicates.

FIG. 12. TEM images with expanded fields of view from of 10 μM hIAPP incubated (a) alone or in the presence of (b) 40 μM cyclo-CHVVGVI (SEQ ID NO:57), (c) 40 μM cyclo-CDLGVFR (SEQ ID NO:68), or (d) 40 μM cyclo-CRCVFSG (SEQ ID NO:69) for 2 h. hIAPP incubations were run in 25 mM sodium phosphate buffer, pH 7.4; see FIG. 13 for hIAPP aggregation kinetics under TEM assay conditions.

FIG. 13. Effect of (a) cyclo-CHVVGVI (SEQ ID NO:57), (b) cyclo-CDLGVFR (SEQ ID NO:68), or (c) cyclo-CRCVFSG (SEQ ID NO:69) on aggregation of 10 μM hIAPP measured by ThT fluorescence. Assay was run in 25 mM sodium phosphate buffer, pH 7.4 at 32° C. under quiescent conditions. Each graph reflects plots of individual values of three technical replicates. The hIAPP alone (0 μM peptide) data is the same in panels (a-c).

FIGS. 14A and 14B. Native mass spectra of 10 μM hIAPP alone (FIG. 14A) or in the presence of 40 μM cyclo-CHVVGI (SEQ ID NO:57) (FIG. 14B). Peak assignments are shown in blue with the theoretical masses given in parentheses. For the full spectra, see FIGS. 15A and 15B.

FIGS. 15A and 15B. Full native mass spectra of 20 μM hIAPP alone (FIG. 15A) or in the presence of 40 μM cyclo-CHVVGI (SEQ ID NO:57) (FIG. 15B). Peak assignments are shown in blue with the theoretical masses given in parentheses.

FIGS. 16A-16C. Negative selection to remove off-target cyclic peptides. (FIG. 16A) Diagram of genetic circuits constituting the negative selection. (FIG. 16B) Cartoon depiction of negative selection strategy using dominant-negative pIII variant pIII-neg. (FIG. 16C) Propagation of SP on negative selection cells that produce pIII-neg upon inhibition of Aβ42-T7n aggregation. The tested SP encode cyclo-CKVWQLL (SEQ ID NO:30) (Aβ42 aggregation inhibitor), cyclo-CEAGQLL (SEQ ID NO:39) (inactive on Aβ42), or kanR (unrelated gene; negative control). Data shown are mean and s.e. of two biological replicates. Fold phage propagation is calculated as the number of phage generated from an infected culture divided by the number of phage (10⁵) used to infect the culture.

FIG. 17. Evaluation of cyclic peptide sequences enriched by selection on hIAPP-T7n expressed from plasmid using the Aβ42-GFP folding reporter. For comparison, positive (CKVWQLL (SEQ ID NO:30)) and negative (CEAGQLL (SEQ ID NO:39)) control cyclic peptides are included. Cyclic peptides are induced at 0.04 mM IPTG. Data reflects mean and s.e. of three biological replicates.

FIG. 18. Replicates of hIAPP aggregation assays with cyclo-CDLGVFR (SEQ ID NO:68) used to calculate t_{lag} values shown in FIG. 6D. Each graph reflects plots of individual values of three technical replicates. The hIAPP only data (0 μM) in (a-d) is the same as in FIG. 7(a-d), respectively.

FIG. 19. Replicates of hIAPP aggregation assays with cyclo-CRCVFSG (SEQ ID NO:69) used to calculate t_{lag} values shown in FIG. 6F. Each graph reflects plots of individual values of three technical replicates. The hIAPP only data (0 μM) in (a-d) is the same as in FIG. 7(a-d), respectively.

FIGS. 20A and 20B. Native mass spectra of 10 μM hIAPP in the presence of 40 μM cyclo-CDLGVFR (SEQ ID NO:68) (FIG. 20A) or cyclo-CRCVFSG (SEQ ID NO:69) (FIG. 20B). Peak assignments are shown in blue with the theoretical masses given in parentheses. For the full spectra, see FIGS. 21A and 21B.

FIGS. 21A and 21B. Full native mass spectra of 10 μM hIAPP in the presence of 40 μM cyclo-CDLGVFR (SEQ ID NO:68) (FIG. 21A) or cyclo-CRCVFSG (SEQ ID NO:69) (FIG. 21B). Peak assignments are shown in blue with the theoretical masses given in parentheses.

FIG. 22. Sequence alignment of pIII-neg proteins and wild-type pIII. ClustalW2 was used to align all pIII-neg proteins against full-length pIII.

### DETAILED DESCRIPTION OF THE INVENTION

One aspect of the invention is directed to selection systems.

The selection systems can comprise a host cell. The term “host cell” as used herein refers to a cell that can be infected by a library phage as described herein, replicate it, and package it into progeny phages that can infect fresh host cells. Exemplary activities of hosts include expression of genes of the phage, replication of the phage genome, and generation of progeny phage particles. One criterion to determine whether a cell is a suitable host cell for a given library phage is to determine whether the cell can support the viral life cycle of a wild-type viral genome from which the library phage is derived. For example, if the library phage is a modified M13 phage genome, as provided in some embodiments described herein, then a suitable host cell would be any cell that can support the wild-type M13 phage life cycle. Suitable host cells for various phages, such as for continuous evolution processes, are well known to those of skill in the art, and the disclosure is not limited in this respect.

The selection systems can further comprise a phage library comprising library phages configured to infect the host cell. “Phage library” as used herein refers to a collection of phages. The collection of phages can typically be contained and intermixed within a single medium or container. The term “phage” is used herein interchangeably with the term “bacteriophage” and refers to a virus that infects bacterial cells. Typically, phages comprise an outer protein capsid enclosing genetic material. The genetic material can be ssRNA, dsRNA, ssDNA, or dsDNA, in either linear or circular form. Phages and phage vectors are well known to those of skill in the art and non-limiting examples of phages that are useful for carrying out the methods provided herein are y (Lysogen), T2, T4, T7, T12, R17, M13, MS2, G4, P1, P2, P4, Phi X174, N4, (6, and 029. In certain embodiments, the phage utilized in the present invention is M13. Such phages are preferably modified to have the characteristics described elsewhere herein. Additional suitable phages and host cells will be apparent to those of skill in the art, and the invention is not limited in this aspect. For an exemplary description of additional suitable phages and host cells, see Elizabeth Kutter and Alexander Sulakvelidze: Bacteriophages: Biology and Applications. CRC Press; 1^{st} edition (December 2004), ISBN: 0849313368; Martha R. J. Clokie and Andrew M. Kropinski: Bacteriophages: Methods and Protocols, Volume 1: Isolation, Characterization, and Interactions (Methods in Molecular Biology) Humana Press; 1^{st} edition (December, 2008), ISBN: 1588296822; Martha R. J. Clokie and Andrew M. Kropinski: Bacteriophages: Methods and Protocols, Volume 2: Molecular and Applied Aspects (Methods in Molecular Biology) Humana Press; 1^{st} edition (December 2008), ISBN: 1603275649; all of which are incorporated herein in their entirety by reference for disclosure of suitable phages and host cells as well as methods and protocols for isolation, culture, and manipulation of such phages.

The library phages are preferably deficient in a replication gene that encodes a replication protein. “Replication protein” as used herein is a protein essential for the production of infectious progeny phages from the host cell when infected with the phage. “Deficient” in this context means that the phage does not have a replication gene that produces a sufficient amount and/or form of the replication protein to support a wild-type level of phage production. “Wild-type level of phage production” refers to a level of phage production from a phage that is equivalent to a library phage of the invention except that it contains a wild-type form of the replication gene. In some versions, the phage can lack a replication gene that expresses the replication protein, such that the replication protein is not expressed at all in the cell. In some versions, the phage can contain a modified form of a replication gene that expresses the replication protein, but the amount and/or a form of the replication protein is insufficient to support the wild-type level of phage production. “Phage production” in this context refers to the extracellular generation of infectious phage particles. The replication protein can be a protein involved in any part of the phage life cycle, including infection, phage genome replication, phage protein expression, phage assembly, phage release, and phage particle stability. Exemplary replication genes which the library phages can be deficient include gill, which encodes pIII; gIV, which encodes pIV; gVI, which encodes pVI; gVII, which encodes pVII; gVIII, which encodes pVIII; and gIX, which encodes pIX. Exemplary pIII sequences include the sequence encoded by the gill sequence provided in the following examples and sequences at least 80%, at least 85%, at least 90%, at least 95%, or at least 99% identical thereto.

Each library phage preferably comprises a library gene configured to express a library peptide in the host cell. “Configured to express” as used herein with respect to a gene refers to a configuration of a gene such that it encodes a particular peptide and has the appropriate genetic elements (e.g., promoter, ribosomal binding site, etc.) to transcribe and translate the coding sequence into the peptide. The term “peptide” as used herein refers to a polymer of amino acid residues linked together by peptide bonds. The term “peptide” is used herein interchangeably with “polypeptide” and “protein.” The library peptide can be a peptide of any size, structure, or function. Typically, a library peptide will be at least three amino acids long. The library peptides can be a fragments of naturally occurring proteins, entire naturally occurring proteins, modified (mutated) forms of naturally occurring proteins or fragments thereof, or any combination thereof.

It is preferred that at least a subset of the library peptides expressed by the library phages comprise different peptide sequences. The number of different peptides expressed by the library phages in a given phage library is referred to herein as the “library size.” In various versions of the invention, the library size can be greater than 10¹, 10², 10³, 10⁴, 10⁵, 10⁶, 10⁷, 10⁸, 10⁹, 10¹⁰, or more. In various versions of the invention, the library size can be up to 10², 10³, 10⁴, 10⁵, 10⁶, 10⁷, 10⁸, 10⁹, 10¹⁰, 10¹¹, 10¹², 10¹³, or more.

The library peptides can comprise linear peptides, cyclic peptides, or a combination thereof. In some versions of the invention, the library peptides comprise cyclic peptides. The cyclic peptides can have a head-to-tail configuration, a side-chain-to-side-chain configuration, or a side-chain-to-terminus configuration. Head-to-tail cyclic peptides have a configuration in which cyclization occurs exclusively through peptide bonds via the amino and carboxy groups of each constituent residue. Side-chain-to-side-chain cyclic peptides have a configuration in which cyclization occurs through side-chain bonding of constituent residue within the peptide. Side-chain-to-terminus cyclic peptides have a configuration in which either the amino or carboxy group of what would otherwise be an N-terminus or C-terminus, respectively, of a linear peptide bonds to a side chain of a constituent residue in the chain. Methods of configuring genes to produce cyclic peptides are known in the art and include, for example, the split-intein circular ligation of peptides and proteins (SICLOPPS) method¹⁵, among others. The library peptides can have any size capable of being made in a host cell. Exemplary sizes include from 2 amino acid residues to 250 amino acid residues or more, such as 2, 3, 4, 5, 6, 7, 8, 9, 10, 11, 12, 13, 14, 15, 20, 25, 30, 35, 40, 45, 50, 75, 100, 125, 150, 175, 200, 225, 250, 275, 300, 350, 400, or any size between any two of the foregoing values.

The selection systems can further comprise a target fusion gene. The target fusion genes of the invention are genes configured to express a target fusion protein, particularly in the host cell. The target fusion protein can comprise a target protein fused to an RNA polymerase unit. The target protein and the RNA polymerase unit can be fused via a linker. The linker can be 1, 2, 3, 4, 5, 6, 7, 8, 9, 10, 15, 20, or more amino acids long. The target fusion gene can be incorporated in the genome of the host cell or can be provided on a non-chromosomal vector (e.g., a plasmid) that can be introduced in the host cell, among other configurations. In some versions, the target fusion gene comprises a promoter that is activated by a transcription factor provided by the library phage, such that the library phage carries a gene configured to express the transcription factor. In some versions, this transcription factor is not produced by the host cell when not infected with the library phage, such that the host cell does not carry a gene configured to express the transcription factor when not infected with the phage.

The RNA polymerase unit can be an entire, functional RNA polymerase or subunit thereof. The RNA polymerase is preferably a DNA-dependent RNA polymerase.

The RNA polymerase in some versions is a promoter-specific RNA polymerase. “Promoter-specific RNA polymerase” refers to an RNA polymerase that specifically binds to a recognition sequence in a cognate promoter, wherein “cognate promoter” refers to a promoter comprising the recognition sequence. Non-limiting examples of promoter-specific RNA polymerases and their cognate promoters include the T7 RNA polymerase and the T7 promoter, the T3 RNA polymerase and the T3 promoter, and the SP6 RNA polymerase and the SP6 promoter, respectively. These and other RNA polymerases and their cognate promoters are well known in the art. Unless specified otherwise, reference to a specific RNA polymerase and cognate promoter (e.g., T7 RNA polymerase and T7 promoter, T3 RNA polymerase and T3 promoter, and SP6 RNA polymerase the SP6 promoter) encompasses the native forms of such elements as well as modified forms derived therefrom that maintain the same functionality. Examples of modified forms include sequence variants and split variants, the latter of which are described below. The RNA polymerase and its cognate promoter are preferably orthogonal to the host cell.

The RNA polymerase in some versions is a multi-subunit RNA polymerase. The multi-subunit RNA polymerase can be a natural multi-subunit RNA polymerase or a split RNA polymerase. Natural multi-subunit RNA polymerases are RNA polymerases that are comprised of multiple subunits in nature. Split RNA polymerases are RNA polymerases that are comprised of a single subunit in nature but can be recombinantly expressed as two or more separate subunits that combine to form a single, functional RNA polymerase. A number of split RNA polymerases are known in the art. Examples include split T7 RNA polymerases among others. See, e.g., Segall-Shapiro et al.⁵⁹ and Shis et al.⁶⁰ An exemplary split T7 RNA polymerase is provided in the following examples as the protein encoded by the “T7n” and “T7c” coding sequences. The exemplary split T7 RNA polymerase can accordingly be used in the present invention, as can split T7 RNA polymerases comprising subunits having at least 80%, least 85%, least 90%, least 95%, or least 99% sequence identity to the T7n subunit and/or the T7c subunit. RNA polymerases such as the T3 RNA polymerase and the SP6 RNA polymerase have high sequence homology to the T7 RNA polymerase and can be split in regions analogous to those in the split T7 RNA polymerases described herein. In preferred versions of the invention, the subunits of the multi-subunit RNA polymerase together exhibit RNA polymerase activity but do not individually exhibit RNA polymerase activity in isolation of each other.

Accordingly, in some versions of the invention, the RNA polymerase unit to which the target protein is fused in the target fusion protein is a first subunit of a multi-subunit RNA polymerase, such as a multi-subunit, promoter-specific RNA polymerase. In the case in which the subunit multi-subunit RNA polymerase is a split T7 RNA polymerase, the target protein can be fused either to the N-terminal subunit of the split RNA polymerase (preferably at the N-terminus thereof) or the C-terminal subunit of the split RNA polymerase (preferably at the C-terminus thereof). The N-terminal subunit of the split RNA polymerase is understood herein to constitute what would be the N-terminal portion of the unsplit RNA polymerase as it exists in nature. The C-terminal subunit of the split RNA polymerase is understood herein to constitute what would be the C-terminal portion of the unsplit RNA polymerase as it exists in nature. An exemplary N-terminal subunit is the peptide encoded by the T7n coding sequence provided in the following examples, or a peptide having at least 80%, least 85%, least 90%, least 95%, or least 99% sequence identity thereto. An exemplary C-terminal subunit is the peptide encoded by the T7c coding sequence provided in the following examples, or a peptide having at least 80%, least 85%, least 90%, least 95%, or least 99% sequence identity thereto.

In various versions, the RNA polymerase unit to which the target protein is fused in the target fusion protein consists of fewer than 250 amino acid residues, fewer than 225 amino acid residues, fewer than 225 amino acid residues, fewer than 200 amino acid residues, fewer than 175 amino acid residues, fewer than 150 amino acid residues, fewer than 100 amino acid residues, fewer than 75 amino acid residues, or fewer than 50 amino acid residues. In various versions, any linker linking the target protein and the RNA polymerase unit in the target fusion protein consists of fewer than 25 amino acid residues, fewer than 20 amino acid residues, fewer than 15 amino acid residues, fewer than 10 amino acid residues, fewer than 9 amino acid residues, fewer than 8 amino acid residues, fewer than 7 amino acid residues, fewer than 6 amino acid residues, fewer than 5 amino acid residues, fewer than 4 amino acid residues, fewer than 3 amino acid residues, or fewer than 2 amino acid residues.

In various versions of the invention, the target protein to which the RNA polymerase unit is fused in the target fusion protein can be any protein of interest suspected of having a structure and/or function affected, either directly or indirectly, by one or more of the library peptides. In some versions, the target protein binds, is predicted to bind, or is suspected of binding to at least one of the library peptides. In some versions, the target protein binds to at least one of the library peptides.

In some versions of the invention, the target protein is a protein that misfolds and/or aggregate and thereby causes the first subunit of the multi-subunit RNA polymerase to misfold and/or aggregate and thereby prevent binding to the one or more additional subunits of the RNA polymerase to result in a functional RNA polymerase. The library peptides in such cases can be peptides that promote, predicted to promote, or are suspected of promoting the folding of the target protein to thereby promote binding of the first subunit to the one or more additional subunits and thereby results in a functional RNA polymerase. The library peptides can promote the folding of the target protein, for example, by binding to it in the folded state to stabilize the folded structure.

In some versions of the invention, the target protein is an aggregation-prone protein. Aggregation-prone proteins are proteins that are intrinsically unstable and spontaneously unfold and aggregate under physiological conditions. Aggregation-prone proteins are well known in the art and are also referred to as “intrinsically disordered proteins.” Positive staining with Thioflavin T (2-[4-(Dimethylamino)phenyl]-3,6-dimethyl-1,3-benzothiazol-3-ium chloride), Thioflavin S (Product No. T1892, Sigma-Aldrich, St. Louis, MO), and Congo Red (sodium salt of 3,3′-([1,1′-biphenyl]-4,4′-diyl)bis(4-aminonaphthalene-1-sulfonic acid)) under physiological conditions are exemplary indicators of aggregation-prone proteins. Aggregation-prone proteins are involved in more than 50 human diseases. See, e.g., ladanza et al. 2018⁶¹. Examples of aggregation-prone proteins include α-synuclein, amyloid beta (Aβ), microtubule-associated protein tau (τ), transthyretin (TTR), antibody light chains, fragments of immunoglobulin light chains, fragments of immunoglobulin heavy chains, full or N-term fragments of serum amyloid A protein (SAA), prion protein (PrP), β2-microglobulin (β2m), huntingtin exon 1 (HttEx1), ABri peptide, Adan peptide, N-term fragments of apolipoprotein A-1 (ApoAI), C-term extended apolipoprotein A-II (ApoAII), apolipoprotein C-II (ApoCII), apolipoprotein C-III (ApoCIII), fragments of gelsolin, lysozyme (LYS), fragments of fibrinogen α-chain, N-term truncated cystatin C, islet amyloid polypeptide (IAPP), calcitronin, atrial natriuretic factor (ANF), N-term fragments of prolactin (PRL), insulin, medin, lactotransferrin, odontogenic ameloblast-associated protein (ODAM), pulmonary surfactant-associated protein C (SP-C), galectin 7 (Gal-7), corneodesmosin (CDSN), C-term fragments of kerato-epithelin (βig-h3), semenogelin-1 (SGI), proteins S100A8/A, enfuvirtide among others (see Guthertz et al. 2022⁶² and Chiti et al. 2017¹).

In some versions of the invention, the target protein is an amyloidogenic protein. Amyloidogenic proteins are proteins that misfold and form amyloid aggregates. Amyloidogenic proteins are well-known in the art. See, e.g., Giasson et al.⁶³. Examples of amyloidogenic proteins include tau protein, alpha-synuclein, amyloid precursor protein, transthyretin, gelsolin, cystatin C, apolipoprotein Al, fibrinogen alpha chain, lactoferrin, beta-2 microglobulin, apolipoprotein A-II, semenogelin I, corneodesmosin, galectin-7, ITM2B, TGFBI, protein C, beta-lactoglobulin, serum amyloid P component, collagen (type XXV), alpha 1, and APPBP2, among others (see Guthertz et al. 2022⁶² and Chiti et al. 2017¹).

In versions employing a multi-subunit RNA polymerase, the selection system of the invention further preferably comprises one or more RNA-polymerase subunit genes configured to express one or more additional subunits of the RNA polymerase. In preferred versions of the invention, the first subunit and the one or more additional subunits are together sufficient to exhibit RNA polymerase activity. In preferred versions of the invention, the first subunit and the one or more additional subunits do not individually exhibit RNA polymerase activity in isolation of each other. The one or more RNA-polymerase subunit genes can be incorporated in the genome of the host cell or can be provided on one or more non-chromosomal vectors (e.g., plasmids) that can be introduced in the host cell, among other configurations.

The selection system of the invention further preferably comprises a selection gene. The selection gene can be incorporated in the genome of the host cell or can be provided on one or more non-chromosomal vectors (e.g., plasmids) that can be introduced in the host cell, among other configurations. The selection genes of the invention preferably comprise a cognate promoter of the RNA polymerase operationally connected to a coding sequence of a selection protein. The selection protein is a protein responsible for promoting either positive or negative selection of the library phage infecting a particular host cell, either by promoting replication of the library phage or by repressing its replication.

The selection protein in some versions comprises a replication protein, such as a replication protein in which the host cell is deficient. Use of a replication in this manner can promote positive selection of the infecting library phage. Exemplary replication proteins that can serve as suitable selection proteins include pIII, which is encoded by the coding sequence of gIII; pIV, which is encoded by the coding sequence of gIV; pVI, which is encoded by the coding sequence of gVI; pVII, which is encoded by the coding sequence of gVII; pVIII, which is encoded by the coding sequence of gVIII; and pIX, which is encoded by the coding sequence of gIX, among others.

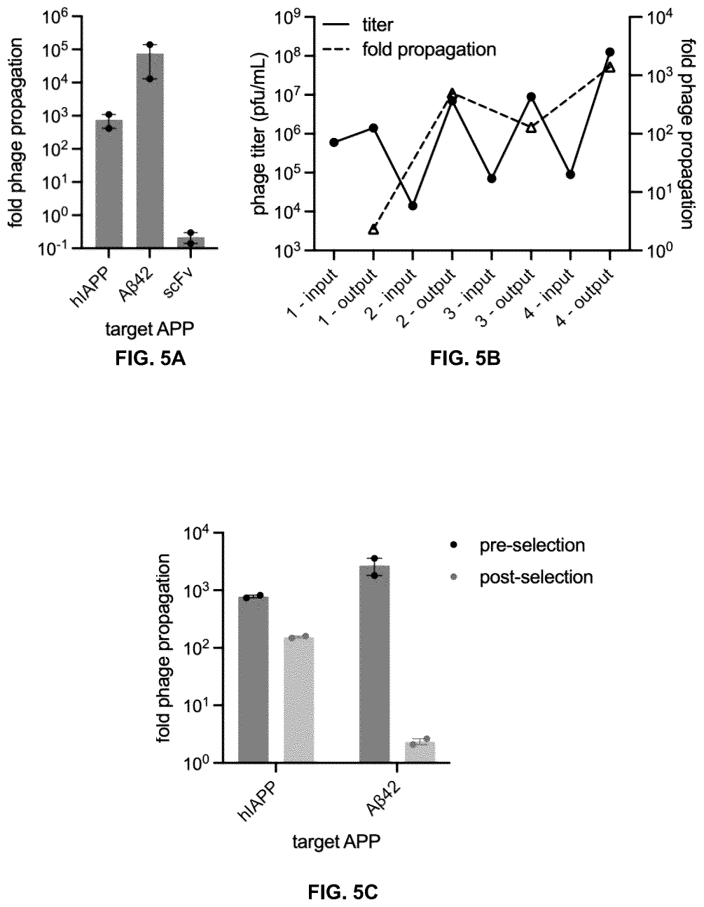

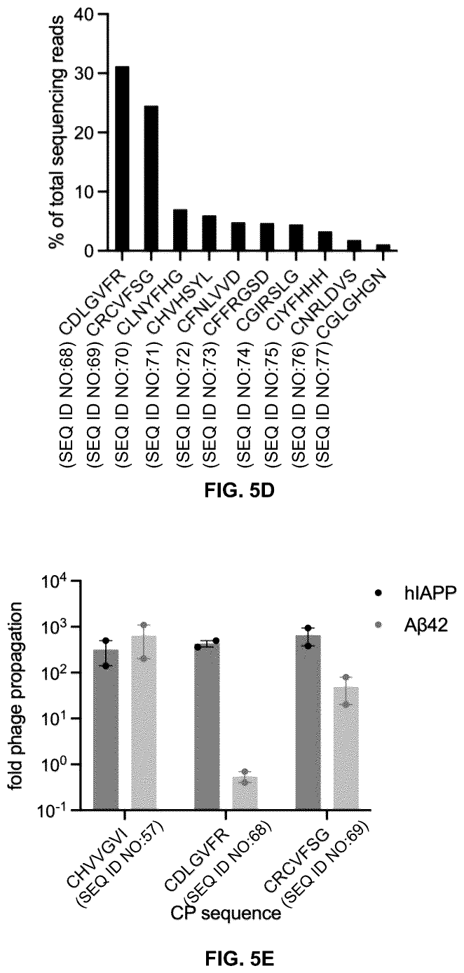

The selection protein in some versions comprises a dominant-negative form of a replication protein, such as a dominant-negative form of a replication protein in which the host cell is deficient. Use of a replication in this manner can promote negative selection of the infecting library phage. Exemplary dominant-negative replication proteins that can serve as suitable selection proteins include dominant-negative forms of pIII, pIV, pVI, pVII, pVIII, and pIX, among others. Examples of a dominant-negative pIII protein is the N-C83 variant, which is a pIII protein comprising an N-C83 domain, which has an internal deletion of 70 amino acids (i.e., amino acids 1-70) from the C-terminal domain of the pIII protein. The amino acid sequence of the N-C83 variant is shown in FIG. 5, which includes other examples of dominant-negative pIII variants. Other exemplary dominant-negative pIII sequences include the sequence encoded by the gIII-neg sequence provided in the following examples and sequences at least 80%, at least 85%, at least 90%, at least 95%, or at least 99% identical thereto. Other examples of dominant-negative replication proteins include dominant-negative mutants of the pIV protein. Other examples of selection proteins that can be used for negative selection include other non-phage conventional counter selection genes.

In versions of the invention employing negative selection with a dominant-negative form of a replication protein as a selection protein, the selection system further preferably comprises a basal replication gene. The basal replication gene is preferably configured to provide minimal expression of a non-dominant-negative version of the replication protein. The basal replication gene can be configured as such by a coding sequence of the non-dominant-negative version of the replication protein being operationally connected to a low-copy number promoter, by being incorporated on a low-copy-number vector, or other configurations. The selection gene and the basal replication gene are preferably provided to the host cell via a vector in trans when negative selection is desired.

The selection systems of the invention can be used in methods of selection. The methods of selection can comprise a first selection. The first selection can comprise contacting a first population of host cells comprising multiple copies of the host cell with the phage library under conditions effective for the library phages to infect the host cells and thereby generate a first population of infected host cells, incubating the first population of infected host cells under conditions effective for production of first progeny phages to thereby produce a first selected phage library comprising the first progeny phages, and harvesting the first selected phage library. The first population of host cells can comprise or be introduced with various genes of the invention suitable for carrying out the selection, including the target fusion gene, the one or more RNA-polymerase subunit genes, and the selection gene. The introduction of these genes can occur simultaneously with the incubating or before or after the incubating. The introduction of these genes can be performed using any suitable method. Harvesting a phage library, such as the first selected phage library, can comprise collecting media in which the first progeny phages are produced. The harvesting can further comprise removing impurities (e.g., cells, cellular debris) through filtration, centrifugation, etc.

The methods of selection can further comprise a second selection. The second selection can comprise contacting a second population of host cells comprising multiple copies of the host cell with the first selected phage library under conditions effective for the first progeny phages to infect the host cells and thereby generate a second population of infected host cells, incubating the second population of infected host cells under conditions effective for production of second progeny phages to thereby produce a second selected phage library comprising the second progeny phages, and harvesting the second selected phage library. The second population of host cells can comprise the same host cells as in the first population of host cells or different host cells. As in the first selection, the second population of host cells can comprise or be introduced with various genes of the invention suitable for carrying out the selection, including the target fusion gene, the one or more RNA-polymerase subunit genes, and the selection gene. The introduction of these genes can occur simultaneously with the incubating or before or after the incubating. The introduction of these genes can be performed using any suitable method.

The methods of selection can further comprise third selections, fourth selections, and so on, each comprising the same or similar steps as described above for the second selection.

In some versions, the first selection, the second selection, and/or any subsequent selections are positive selections. For example, the selection steps can be performed with a positive selection protein, such as a replication protein. In some versions, the first selection, the second selection, and/or any subsequent selections are negative selections. For example, the selection steps can be performed with a negative selection protein, such as a dominant-negative form of a replication protein or some other negative selection protein. In some versions, one or more positive selections are performed and then followed by one or more negative selections. In such versions, the target protein in the one or more negative selections is preferably different than the target protein employed in the one or more preceding positive selections.

Another aspect of the invention is directed to recombinant peptides. The recombinant peptides can comprise any library peptide of the invention. In some versions, the recombinant peptides of the invention comprise a library peptide of the invention that has been selected for reducing aggregation of an aggregation-prone protein using the selection methods of the invention.

In some versions, the recombinant peptides comprise cyclic peptides. The cyclic peptides in some versions comprise head-to-tail cyclic peptides comprising a sequence selected from the group consisting of DLGVFRXₙ (SEQ ID NO:1), RCVFSGXₙ (SEQ ID NO:2), HVVGVIXₙ (SEQ ID NO:3), HVHSYLXₙ (SEQ ID NO:4), LNYFHGXₙ (SEQ ID NO:5), YILSIGXₙ (SEQ ID NO:6), CGLYNIXₙ (SEQ ID NO:7), CHSFFRXₙ (SEQ ID NO:8), GIRSLGXₙ (SEQ ID NO:9), ISCHYGXₙ (SEQ ID NO:10), IYFHHHXₙ (SEQ ID NO:11), VSYILLXₙ (SEQ ID NO:12), FNLVVDXₙ (SEQ ID NO:13), FFRGSDXₙ (SEQ ID NO:14), NRLDVSXₙ (SEQ ID NO:15), GLGHGNXₙ (SEQ ID NO:16), RVWQLCXₙ (SEQ ID NO:17), IVWQLCXₙ (SEQ ID NO:18), KVWQLAXₙ (SEQ ID NO:19), RVWCARXₙ (SEQ ID NO:20), RVYQVLXₙ (SEQ ID NO:21), QVWSAAXₙ (SEQ ID NO:22), RVSQVLXₙ (SEQ ID NO:23), KVWGGLXₙ (SEQ ID NO:24), RVYPVLXₙ (SEQ ID NO:25), QVWSARXₙ (SEQ ID NO:26), QVWCARXₙ (SEQ ID NO:27), TVWTCLXₙ (SEQ ID NO:28), and KVYTAPXₙ (SEQ ID NO:29), wherein X is any amino acid and n is any integer. In versions, n is an integer from 0-30. In some versions, n is an integer such as 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or an integer between any two of the foregoing values. In some versions, at least one X is selected from the group consisting of cysteine, serine, and threonine. In some versions, X is 1. In some versions, X is 1 and the X is selected from the group consisting of cysteine, serine, and threonine. In some versions, X is 1 and the X is cysteine. In some versions, the most N-terminal X of Xₙ is selected from the group consisting of cysteine, serine, and threonine. In some versions, the most N-terminal X of Xₙ is cysteine.

In some versions, the cyclic peptides of the invention are generated using a method referred to in the art as SICLOPPS (Valentine et al. 2018⁶⁴, Tavassoli 2017⁶⁵). SICLOPPS employs a cysteine, serine, or threonine as the first amino acid of an extein forming the final cyclic peptide (Valentine et al. 2018⁶⁴). Beyond this, there are no other limits on the number or identity of amino acids in the target peptide, allowing cyclic peptides of various sizes and sequences to be assembled (Tavassoli 2017⁶⁵). In some versions, the cyclic peptides of the invention are chemically synthesized, thereby removing the requirement for a cysteine, serine, or threonine as the first amino acid of the extein.

In some versions, the cyclic peptides can be isolated cyclic peptides.

The DLGVFRXₙ (SEQ ID NO:1), RCVFSGXₙ (SEQ ID NO:2), HVVGVIXₙ (SEQ ID NO:3), HVHSYLXₙ (SEQ ID NO:4), LNYFHGXₙ (SEQ ID NO:5), YILSIGXₙ (SEQ ID NO:6), CGLYNIXₙ (SEQ ID NO:7), CHSFFRXₙ (SEQ ID NO:8), GIRSLGXₙ (SEQ ID NO:9), ISCHYGXₙ (SEQ ID NO:10), IYFHHHXₙ (SEQ ID NO:11), VSYILLXₙ (SEQ ID NO:12), FNLWDXₙ (SEQ ID NO:13), FFRGSDXₙ (SEQ ID NO:14), NRLDVSXₙ (SEQ ID NO:15), and GLGHGNXₙ (SEQ ID NO:16) cyclic peptides of the invention are particularly useful for reducing aggregation of human islet amyloid polypeptide. The RVWQLCXₙ (SEQ ID NO:17), IVWQLCXₙ (SEQ ID NO:18), and KVWQLAXₙ (SEQ ID NO:19, RVWCARXₙ (SEQ ID NO:20), RVYQVLXₙ (SEQ ID NO:21), QVWSAAXₙ (SEQ ID NO:22), RVSQVLXₙ (SEQ ID NO:23), KVWGGLXₙ (SEQ ID NO:24), RVYPVLXₙ (SEQ ID NO:25), QVWSARXₙ (SEQ ID NO:26), QVWCARXₙ (SEQ ID NO:27), TVWTCLXₙ (SEQ ID NO:28), and KVYTAPXₙ (SEQ ID NO:29) cyclic peptides of the invention are particularly useful for reducing the aggregation of amyloid-β42.

Another aspect of the invention is directed to methods of reducing aggregation of an aggregation-prone protein. The methods can comprise contacting an aggregation-prone protein with a peptide of the invention. The contacting can be performed in vitro or in vivo. If performed in vivo, the peptide can be administered to a subject comprising the aggregation-prone protein using any suitable method. The peptide in such methods can comprise a library peptide that has been selected for reducing aggregation of the aggregation-prone protein using the selection methods of the invention. In some versions, the aggregation-prone protein comprises human islet amyloid polypeptide, and the cyclic peptide is selected from the group consisting of DLGVFRXₙ (SEQ ID NO:1), RCVFSGXₙ (SEQ ID NO:2), HVVGVIXₙ (SEQ ID NO:3), HVHSYLXₙ (SEQ ID NO:4), LNYFHGXₙ (SEQ ID NO:5), YILSIGXₙ (SEQ ID NO:6), CGLYNIXₙ (SEQ ID NO:7), CHSFFRXₙ (SEQ ID NO:8), GIRSLGXₙ (SEQ ID NO:9), ISCHYGXₙ (SEQ ID NO:10), IYFHHHXₙ (SEQ ID NO:11), VSYILLXₙ (SEQ ID NO:12), FNLWDXₙ (SEQ ID NO:13), FFRGSDXₙ (SEQ ID NO:14), NRLDVSXₙ (SEQ ID NO:15), and GLGHGNXₙ (SEQ ID NO:16), wherein X is any amino acid and n is any integer. In some versions, the contacting the human islet amyloid polypeptide with the cyclic peptide is performed in a subject with type 2 diabetes.

In some versions, the aggregation-prone protein comprises amyloid-β42, and the cyclic peptide is selected from the group consisting of RVWQLCXₙ (SEQ ID NO:17), IVWQLCXₙ (SEQ ID NO:18), and KVWQLAXₙ (SEQ ID NO:19, RVWCARXₙ (SEQ ID NO:20), RVYQVLXₙ (SEQ ID NO:21), QVWSAAXₙ (SEQ ID NO:22), RVSQVLXₙ (SEQ ID NO:23), KVWGGLXₙ (SEQ ID NO:24), RVYPVLXₙ (SEQ ID NO:25), QVWSARXₙ (SEQ ID NO:26), QVWCARXₙ (SEQ ID NO:27), TVWTCLXₙ (SEQ ID NO:28), and KVYTAPXₙ (SEQ ID NO:29), wherein X is any amino acid and n is any integer. In some versions, the contacting the amyloid-β42 with the cyclic peptide is performed in a subject with Alzheimer's disease.

“Gene” refers to a nucleic acid sequence capable of producing a gene product and may include such genetic elements as a coding sequence together with any other genetic elements required for transcription and/or translation of the coding sequence. Such genetic elements may include a promoter, an enhancer, and/or a ribosome binding site (RBS), among others. In some versions, multiple genes are configured in an operon, in which multiple coding sequences are operationally connected to a single promoter. Each coding sequence and promoter pair in such instances are considered herein to constitute separate genes, despite comprising the same promoter.

“Gene product” refers to products such as a polypeptide or an mRNA encoded and produced by a particular gene.

“Operationally connected” refers to a relationship between two genetic elements (e.g., a promoter and coding sequence), in which one of the genetic elements controls or affects the activity of the other genetic element.

“Endogenous” used in reference to a genetic element means that the genetic element is native to the cell in which it is disposed.

“Exogenous” used in reference to a genetic element means that the genetic element is not native to the cell in which it is disposed.

“Recombinant” as used herein with reference to nucleic acid molecules or polypeptides refers to nucleic acid molecules or polypeptides having a non-natural nucleic acid or polypeptide sequence, respectively. “Recombinant” as used herein with reference to a gene refers to a gene having a non-natural nucleic acid sequence, is exogenous, or is endogenous to a given cell but is disposed within the cell (e.g., within the cell's genome) at a locus different from the native form of the gene. “Recombinant” as used herein with reference to a cell refers to a cell that contains a recombinant nucleic acid molecule, polypeptide, or gene. Any gene, polypeptide, or protein described herein can be a recombinant gene, polypeptide, or protein.

A “homologous” gene or protein is a gene or protein inherited in two species from a common ancestor. While homologous genes or proteins can be similar in sequence, similar sequences are not necessarily homologous.

The terms “identical” or “percent identity”, in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described herein (or other algorithms available to persons of skill) or by visual inspection. For sequence comparison and identity determination, one sequence typically acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence based on the designated program parameters. A typical reference sequence of the invention is any nucleic acid or amino acid sequence described herein. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see Current Protocols in Molecular Biology, F. M. Ausubel et al., eds., Current Protocols, a joint venture between Greene Publishing Associates, Inc. and John Wiley & Sons, Inc., (supplemented through 2008)). One example of an algorithm that is suitable for determining percent sequence identity and sequence similarity for purposes of defining homologs is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915). In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001. The above-described techniques are useful in determining sequence identity of sequences described herein.

Various methods for introducing genetic modifications are well known in the art and include homologous recombination, among other mechanisms. See, e.g., Green et al., Molecular Cloning: A laboratory manual, 4^{th} ed., Cold Spring Harbor Laboratory Press (2012) and Sambrook et al., Molecular Cloning: A Laboratory Manual, 3^{rd} ed., Cold Spring Harbor Laboratory Press (2001).

The genes of the invention can be codon-optimized for the particular microorganism in which they are introduced. Codon optimization can be performed for any nucleic acid by a number of programs, including “GENEGPS”-brand expression optimization algorithm by DNA 2.0 (Menlo Park, CA), “GENEOPTIMIZER”-brand gene optimization software by Life Technologies (Grand Island, NY), and “OPTIMUMGENE”-brand gene design system by GenScript (Piscataway, NJ). Other codon optimization programs or services are well known and commercially available.

The term “introduce” used with reference to introducing a gene or other element into a cell refers to transferring the gene from outside of the cell to inside of the cell. Such introduction can be performed using any method in the art. Methods for introduction include but are not limited to protoplast fusion, transfection, transformation, conjugation, and transduction (see, e.g., Ferrari et al., Genetics, in Hardwood et al, (eds.), Bacillus, Plenum Publishing Corp., pp. 57-72, 1989).

The term “isolated” or “purified” means a material that is removed from its original environment, for example, the natural environment if it is naturally occurring, or a cultivation broth if it is produced in a recombinant host cell cultivation medium. A material is said to be “purified” when it is present in a particular composition in a higher concentration than the concentration that exists prior to the purification step(s).

U.S. Pat. Nos. 10,179,911 and 11,624,130 are incorporated herein by reference.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Rapid Discovery of Cyclic Peptide Protein Aggregation Inhibitors by Continuous Selection

### Summary

We report a new platform for the rapid phenotypic selection of protein aggregation inhibitors from genetically encoded cyclic peptide libraries in E. coli based on phage-assisted continuous evolution (PACE). Here, we developed a PACE-compatible selection for protein aggregation inhibition and employed it to identify cyclic peptides that suppress amyloid-β42 (Aβ42) and human islet amyloid polypeptide (hIAPP) aggregation. Additionally, we integrated a negative selection that removes false positives and off-target hits, significantly improving cyclic peptide selectivity. We show that selected inhibitors are active when chemically re-synthesized in in vitro assays. Our platform provides a powerful new approach for the rapid discovery of cyclic peptide inhibitors of protein aggregation and may serve as the basis for the future evolution of cyclic peptides with a broad spectrum of inhibitory activities.

### Introduction

We have developed a new platform for selecting cyclic peptide protein aggregation inhibitors based on phage-assisted continuous evolution (PACE). By linking cyclic peptide activity to phage reproduction, we can perform 1-2 rounds of selection per day, leading to rapid discovery of active sequences using only standard molecular biology techniques and equipment. We demonstrate the utility of our system by using it to identify cyclic peptide sequences that inhibit the aggregation of disease-associated proteins amyloid-β42 (Aβ42) and human islet amyloid polypeptide (hIAPP). We also report the implementation of a negative selection that purges hits with off-target activity, resulting in selective cyclic peptide sequences. Finally, we show that our identified hIAPP inhibitors are active in in vitro amyloid formation assays. We envision that the reported system will improve the speed and convenience of cellular cyclic peptide selections and complement existing strategies for identifying inhibitors of disease-associated protein aggregation.

### Results

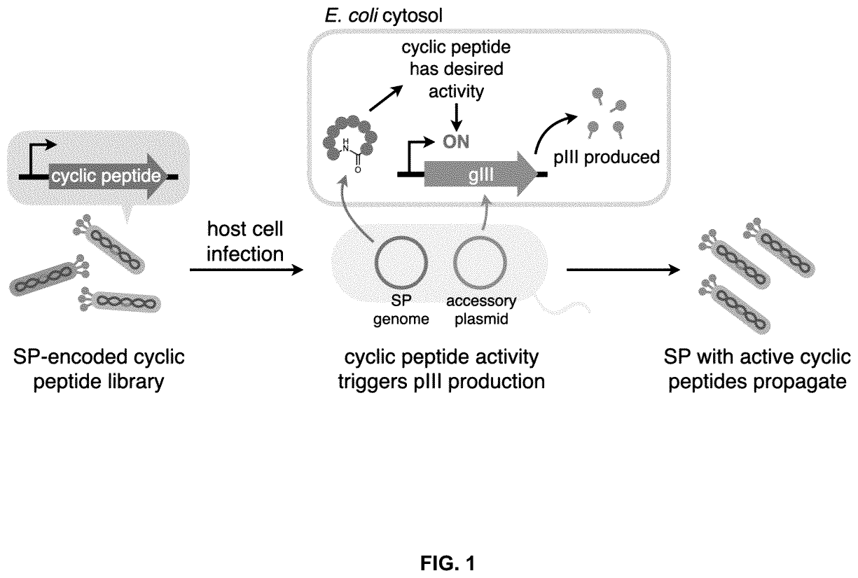

We envisioned a general strategy for phage-assisted cyclic peptide discovery, shown in FIG. 1. We start with an selection phage (SP)-encoded cyclic peptide library generated by standard cloning techniques. This pool of SP is used to infect E. coli host cells with an accessory plasmid that links desired cyclic peptide activity with protein III (pill) expression. pill, which is encoded by gill, is essential for both infection of and viral escape from the E. coli host cell. Upon infection, the E. coli biosynthesizes all SP-encoded genes, including the cyclic peptide. If the cyclic peptide possesses the activity under selection, it triggers pill expression from the host cell, enabling phage propagation. Thus, only SP that carry active cyclic peptides enrich in the population.

### Development of a PACE-Compatible Selection for Protein Aggregation Inhibitors

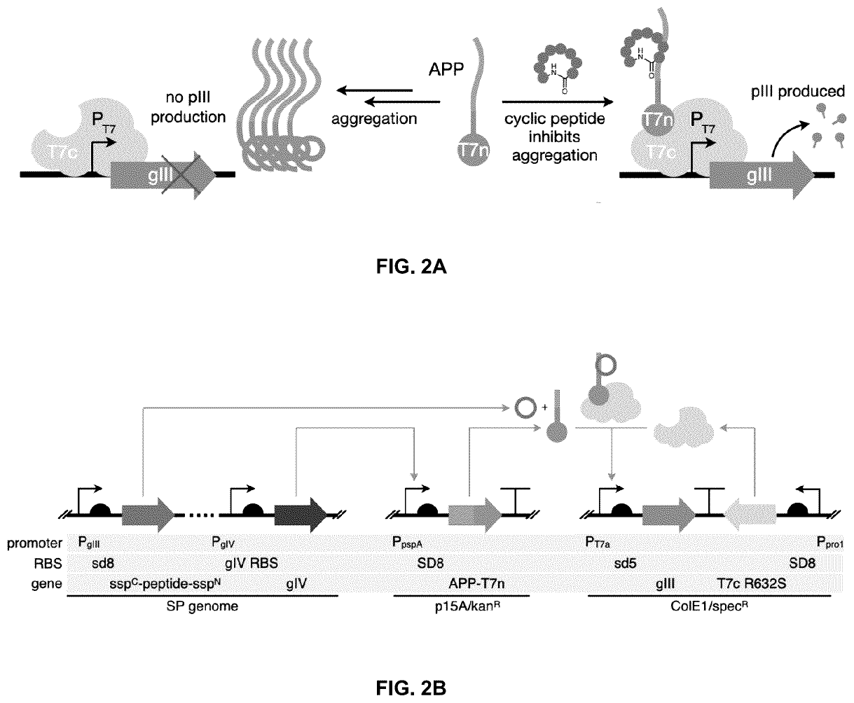

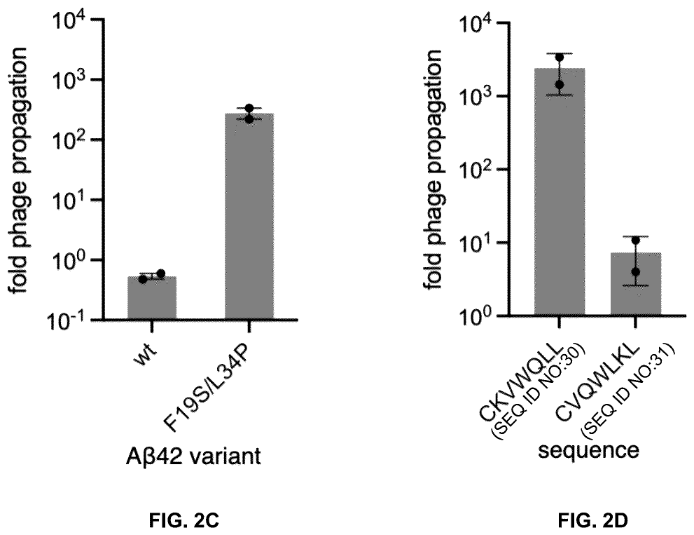

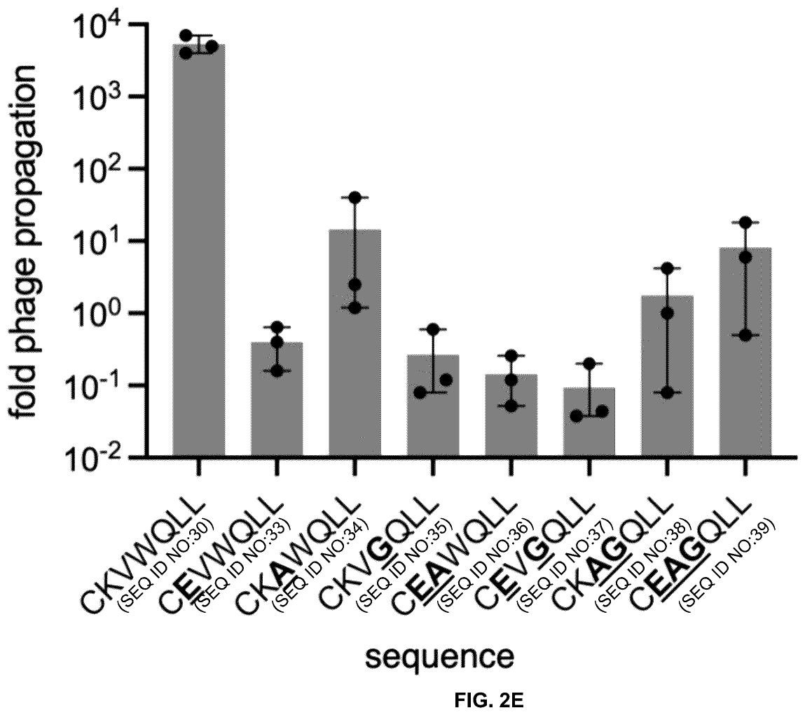

Previous strategies for selecting protein misfolding inhibitors in cells have utilized cell viability^{4,19,29,30} or signal from fluorescent protein fusions.^{31,32} These approaches cannot translate to PACE, which requires the selected activity to be coupled to gIII expression. Therefore, we turned to a split-T7 RNA polymerase (T7 RNAP) reporter used to evolve proteins with improved soluble expression.³³ In our new selection, host E. coli express an aggregation-prone protein (APP) fused to the N-terminal half of split-T7 RNAP (T7n). When the APP-T7n fusion is translated, it misfolds due to the APP, preventing T7n from associating with the C-terminal half of split-T7 RNAP (T7c; FIG. 2A). If a SP-encoded inhibitor binds to and prevents APP aggregation, the fused T7n will remain folded and join with T7c to produce full-length T7 RNAP. The reconstituted T7 RNAP then transcribes gIII, which we place under control of the T7 promoter (P_{T7}), producing pill and allowing the SP to reproduce (FIGS. 2A and 2B).

We initially targeted amyloid-β42 (Aβ42) peptide, which is implicated in Alzheimer's Disease, as the APP. Our choice was motivated by the recent identification of a cyclic peptide inhibitor of Aβ42 aggregation, cyclo-CKVWQLL^{31,32} (SEQ ID NO:30) that would provide a valuable control for optimizing and benchmarking our system. When fused to T7n, Aβ42 should rapidly aggregate and prevent T7 RNAP reconstitution. Accordingly, this fusion produces low gIII transcription from P_{T7}, and SP carrying a control gene (kanR) reproduce poorly on host cells encoding the selection circuit (FIG. 2C). For comparison, we tested a fusion of a non-aggregating Aβ42 mutant (F19S/L34P),³⁴ which gave rise to a ˜500-fold increase in propagation of kanR SP (FIG. 2C). These results suggest that the split-T7 RNAP folding reporter can translate the degree of Aβ42 aggregation to changes in gIII transcription.

### Selection of SP-Encoded SICLOPPS Cyclic Peptides

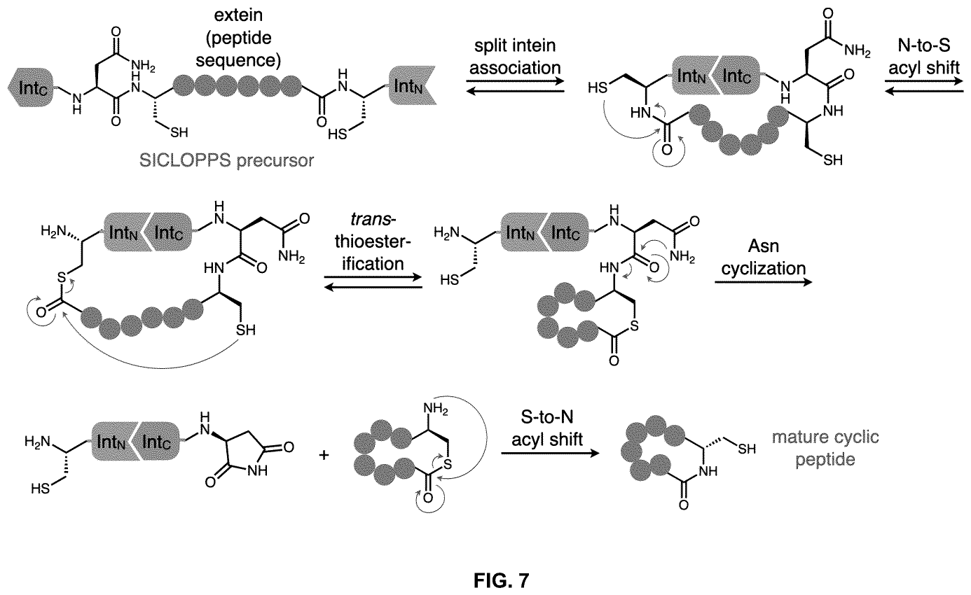

Next, we determined if our selection could discriminate between SP encoding cyclic peptides that inhibit Aβ42 aggregation and SP carrying inactive cyclic peptides. We used the SICLOPPS method to biosynthesize cyclic peptides that can be selected in E. coli.¹⁵ In SICLOPPS, a precursor fusion consisting of the C-terminal half of the Ssp DnaE split intein, the peptide sequence to be cyclized, and the N-terminal half of the same split intein undergoes intein splicing after translation to produce head-to-tail cyclized peptides (FIG. 7). To benchmark our system, we used positive control sequence cyclo-CKVWQLL (SEQ ID NO:30), identified from a SICLOPPS cyclic peptide library to inhibit intracellular Aβ42-GFP aggregation and shown to be active when chemically re-synthesized in both in vitro and C. elegans Aβ42 aggregation models.³¹

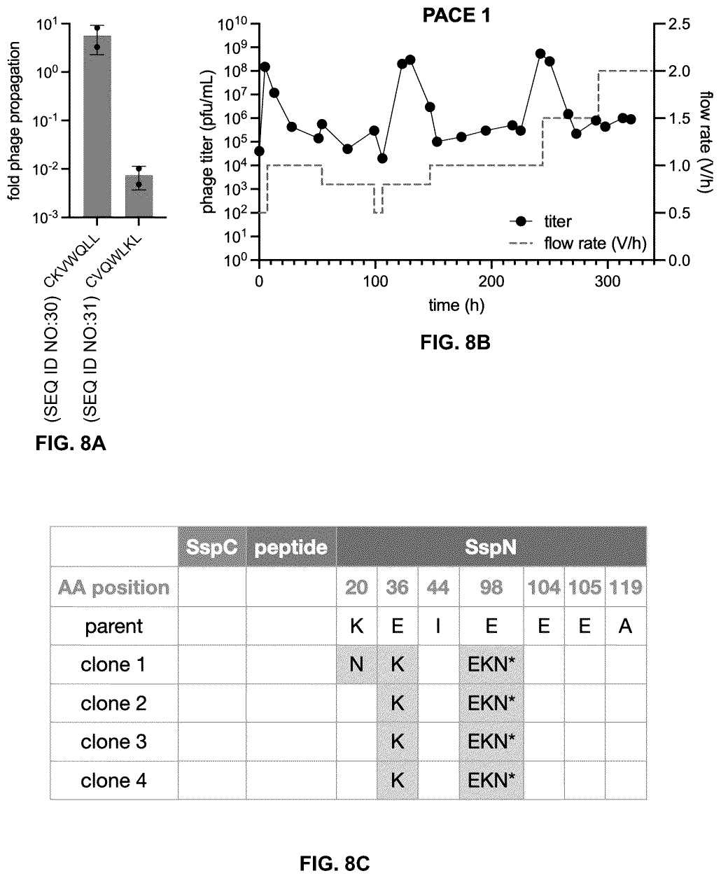

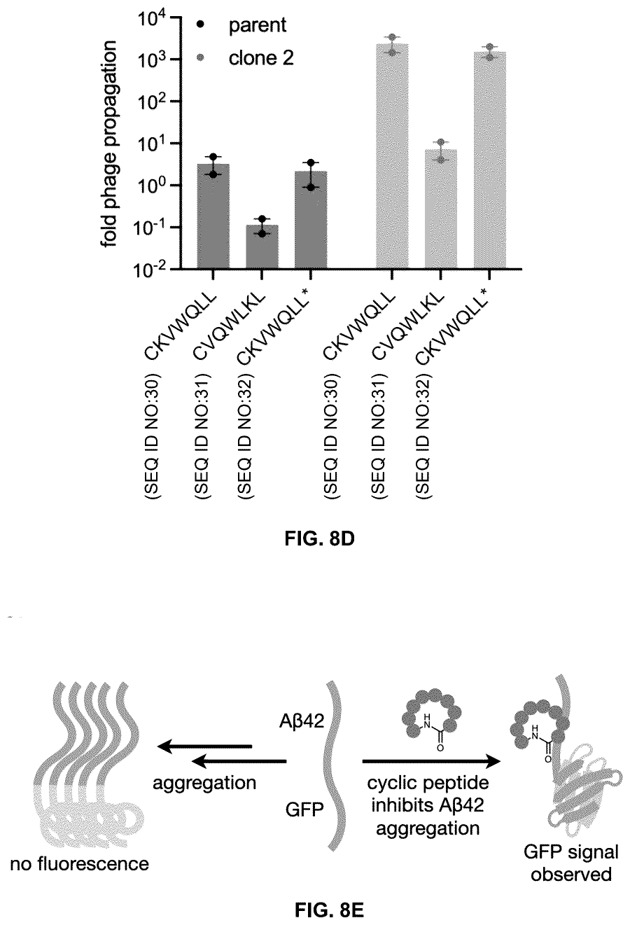

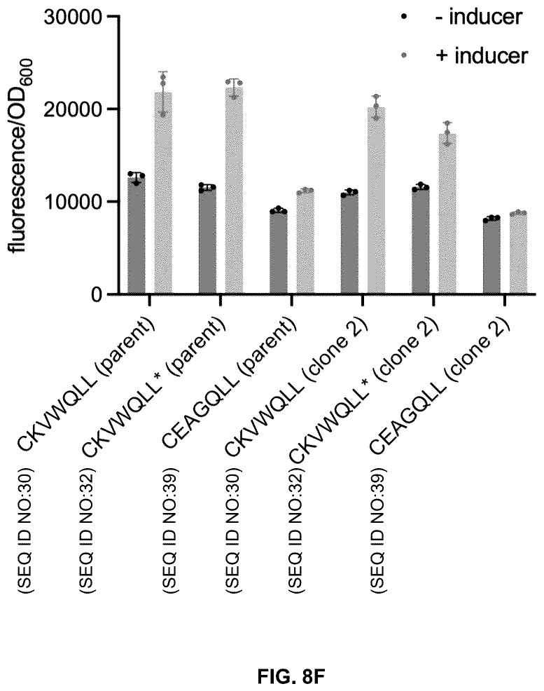

We measured the reproduction of SP carrying SICLOPPS precursors for cyclo-CKVWQLL (SEQ ID NO:30) (“CKVWQLL SP”) or a scrambled control cyclo-CVQWLKL (SEQ ID NO:31) (“CVQWLKL SP”) on E. coli encoding the Aβ42-T7n selection (FIG. 2B). CKVWQLL SP exhibited significantly increased (˜500-fold) proliferation compared to SP carrying the scrambled control (FIG. 8A). However, we were concerned that the low overall phage amplifications observed (0.01- to 10-fold) could preclude selection of active cyclic peptides from a library. We thought that cyclic peptide maturation might not be fast enough to trigger pIII production during the SP life cycle (˜15 min). Alternatively, SP backbone optimization could be required to coordinate the phage life cycle with our selection, which has been observed previously and can impose a prerequisite for implementing new PACE selections.^{27,35,36} To improve the overall fitness of cyclic peptide-encoding phage, we evolved the parent CKVWQLL SP on Aβ42-T7n selection cells in PACE (FIG. 8B). We chose SP clone 2 (FIG. 8C) for downstream experiments. This evolved SP exhibited significantly increased overall proliferation compared to the parent (FIG. 8D) yet maintained excellent (˜300-fold) discrimination between CKVWQLL SP and scrambled control (FIG. 2D). Installing point mutations at residues important for CKVWQLL (SEQ ID NO:30) activity³¹ produced decreases in SP propagation, further supporting dependence of phage fitness on cyclic peptide sequence (FIG. 2E).

Cyclic peptide formation by SICLOPPS occurs through intein splicing. We tested whether phage propagation was splicing dependent using CKVWQLL SP where the first cysteine in the N-terminal intein was mutated to alanine, preventing formation of the first splicing intermediate (FIG. 7). Unexpectedly, intein inactivation did not impact CKVWQLL SP propagation (FIG. 8D), suggesting that both intein-bound and mature cyclic peptide forms of this sequence are active. The lack of splicing dependence could not be explained by our evolved SP because the unevolved SP also exhibited splicing-independent activity (FIG. 8D). Additionally, it was not an artifact of our phage-based selection, as we also observed it in experiments testing plasmid-expressed SICLOPPS precursors on an Aβ42-GFP folding reporter analogous to the one used to identify the CKVWQLL sequence (SEQ ID NO:30) (FIGS. 8E and 8F).³¹ This suggests that, when selecting SICLOPPS libraries, the species undergoing selection may exist as a mixture of pre-splice, splicing intermediate, and spliced products. However, SICLOPPS libraries have repeatedly produced cyclic peptides that function when chemically re-synthesized and tested in orthogonal assays,^{20,21,37,38} suggesting that this ambiguity does not prevent the discovery of active hits.

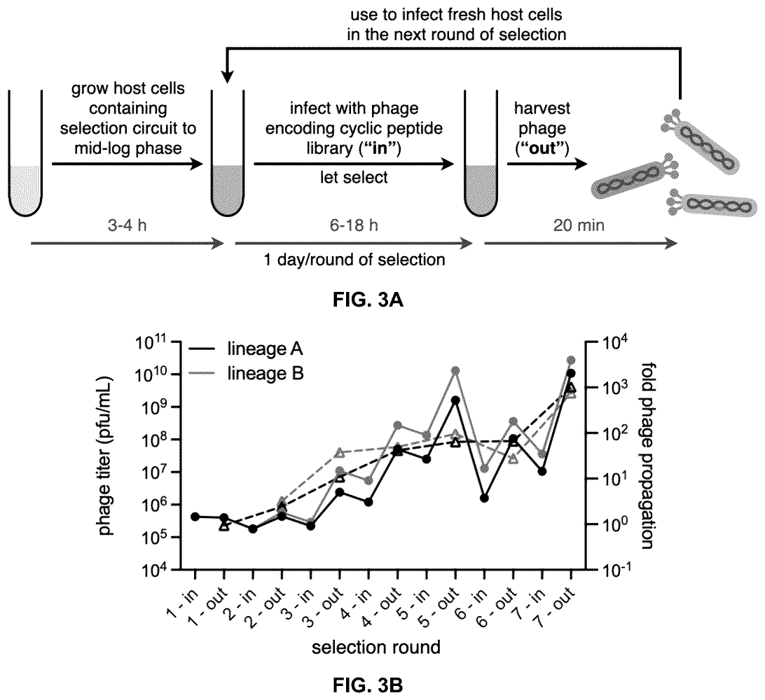

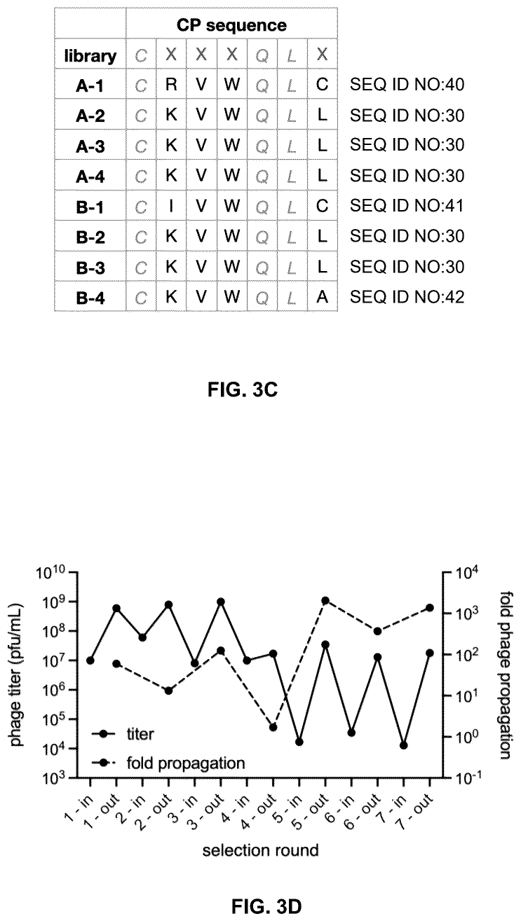

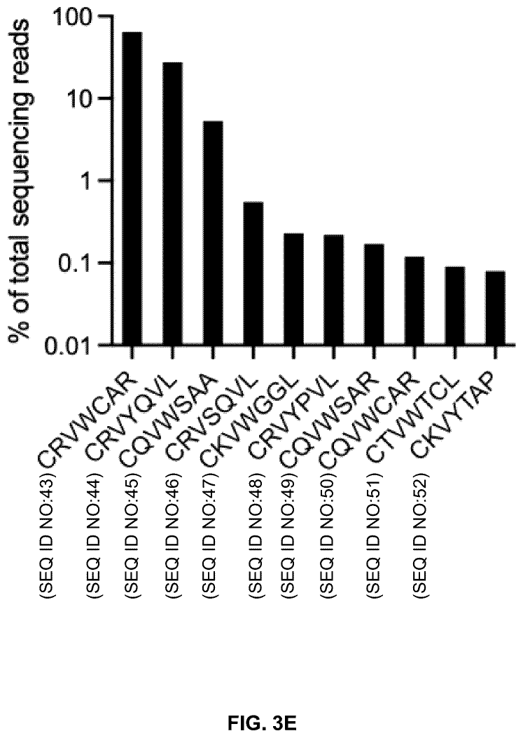

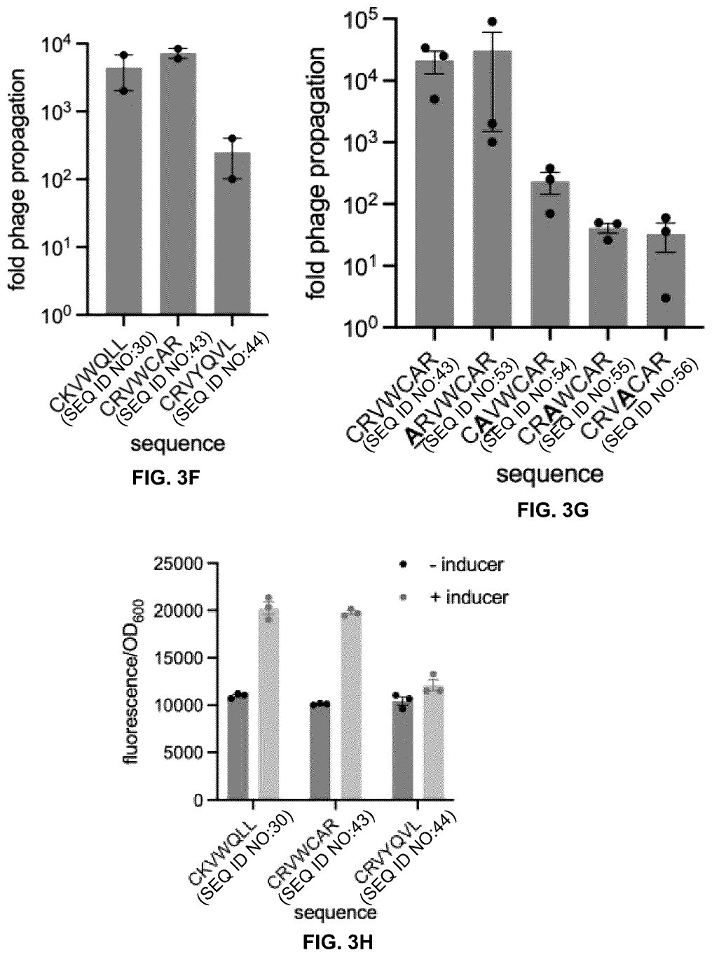

We performed a mock selection using a SP library where the four residues in CKVWQLL (SEQ ID NO:30) most critical for activity³¹ were diversified by saturation mutagenesis. We subjected the resulting CX₃QLX (X=amino acid encoded by NNK codons) library to PANCS-style selection²⁶ using host cells encoding the Aβ42-T7n selection circuit (FIG. 3A). After the first round, we split the SP pool into two separate lineages and selected them in parallel. Initially, we diluted the SP 2-fold per round and gradually increased the dilution factor to 10-fold in later rounds. After seven rounds, we observed a ˜500-fold increase in SP pool fitness (measured by fold phage propagation on the selection cells), suggesting enrichment of active cyclic peptide sequences (FIG. 3B). Sanger sequencing of four clonal SP from each lineage revealed that the phage had either rediscovered CKVWQLL (SEQ ID NO:30) or enriched highly similar sequences (FIG. 3C). Together, these results suggest that we can select for active cyclic peptide sequences from an SP-encoded library using our system.

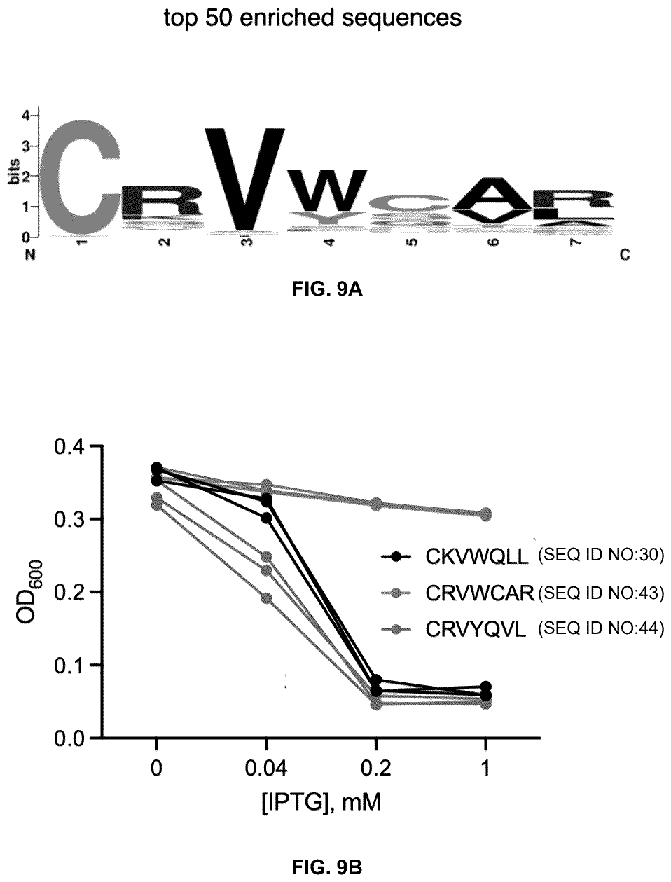

Next, we tested whether we could select active cyclic peptides from a library of randomized sequences by subjecting a cyclo-CX₆ SP library (X=NNK encoded amino acid) to PANCS²⁶ using host cells encoding the Aβ42-T7n selection circuit (FIG. 3A). A total of ˜3×10⁶ transformants was selected over seven rounds, over which we observed an increase in SP pool fitness (measured by fold phage propagation on the selection cells), suggesting enrichment of active cyclic peptide sequences (FIG. 3D). Deep sequencing revealed that the final SP pool had strongly converged, with the top sequence (CRVWCAR (SEQ ID NO:43)) accounting for ˜65% of total sequencing reads (FIG. 3E). Alignment of the top 50 sequences (FIG. 9A) revealed high similarity to cyclo-CKVWQLL (SEQ ID NO:30), while the top sequence (CRVWCAR (SEQ ID NO:43)) bears strong resemblance to sequences highly enriched by the selection that identified cyclo-CKVWQLL (SEQ ID NO:30) in a prior study (e.g., CRVWCAL (SEQ ID NO:) and CRVWQAL (SEQ ID NO:)).²⁰

We measured the fitness of clonal phage encoding the top two sequences, CRVWCAR (SEQ ID NO:43) and CRVYQVL (SEQ ID NO:44), on the Aβ42-T7n selection (FIG. 3F). SP carrying CRVWCAR (SEQ ID NO:43) exhibited robust propagation, comparable to that of CKVWQLL SP, while SP bearing CRVYQVL (SEQ ID NO:44) displayed more modest fitness. We also probed the sequence dependence of CRVWCAR (SEQ ID NO:43) through alanine substitutions (FIG. 3G). Mutation of residues 2-4 to alanine greatly diminished activity, consistent with the strong enrichment of R, V, and W at these positions by our selection (FIG. 9A). However, substitution of the first cysteine with alanine, which prevents SICLOPPS splicing, had no discernable effect on Aβ42-T7n inhibition. This is consistent with our observations that CKVWQLL (SEQ ID NO:30) activity is also not splicing-dependent (FIGS. 8D-8F). Finally, we compared the activity of our sequences with CKVWQLL (SEQ ID NO:30) in the Aβ42-GFP folding reporter assay (FIG. 3H). CRVWCAR (SEQ ID NO:43) had comparable activity to CKVWQLL (SEQ ID NO:30), while CRVYQVL (SEQ ID NO:44) appeared inactive. However, induction of the CRVYQVL (SEQ ID NO:44) construct greatly decreased cell growth (FIG. 9B), which may convolute rescue of Aβ42-GFP fluorescence.

Together, these results suggest that our system can select for active cyclic peptide sequences from SP-encoded libraries.

Selection of Cyclic Peptide hIAPP Aggregation Inhibitors

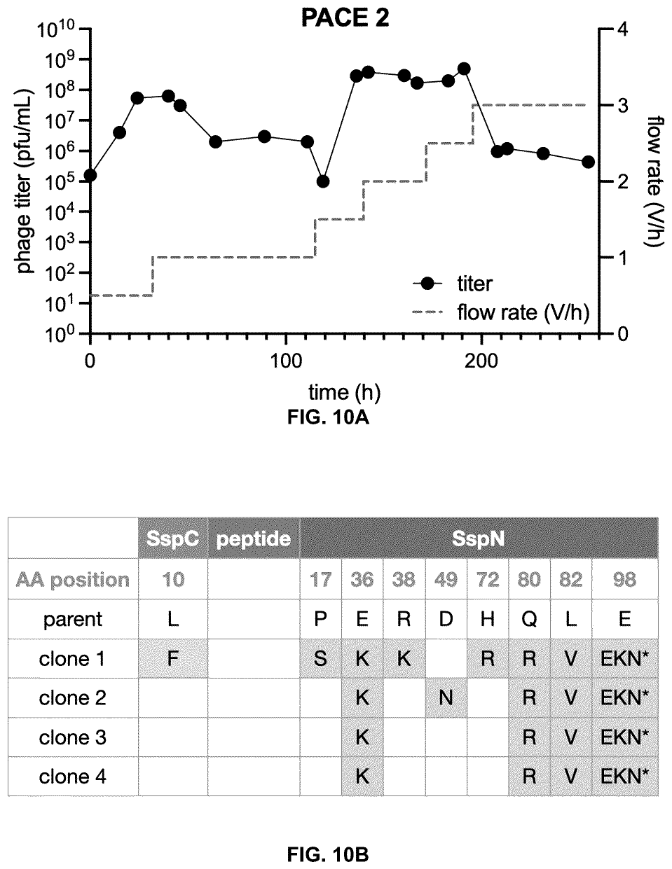

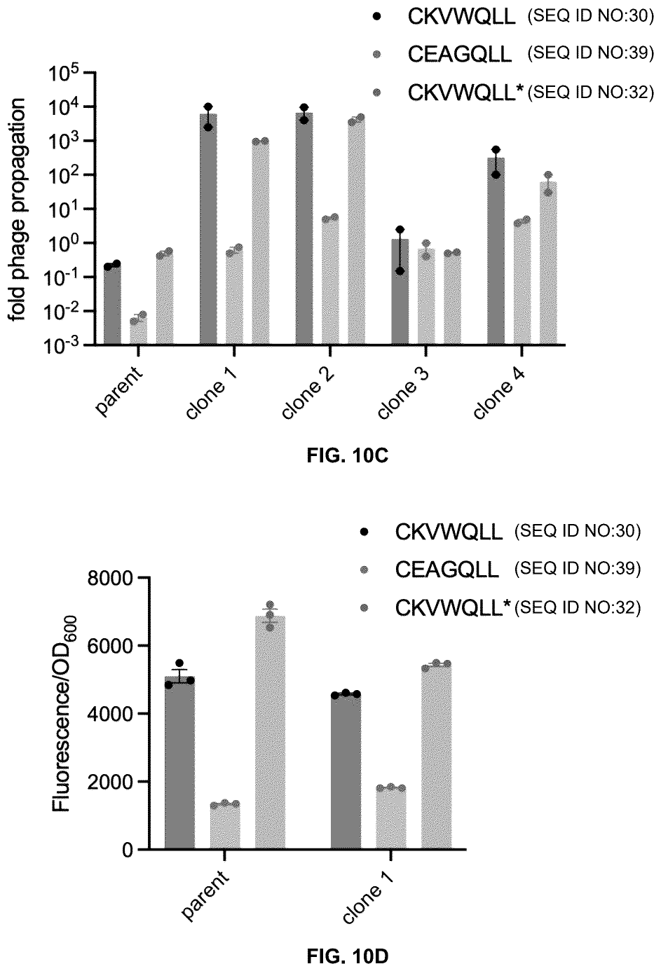

Next, we examined if our system could discover inhibitors of other disease-associated APPs. We targeted human islet amyloid polypeptide (hIAPP), which forms amyloid deposits in patients with type-2 diabetes that are hypothesized to contribute to R-cell dysfunction.⁴⁰ To create a selection for hIAPP aggregation inhibitors, we simply cloned hIAPP in place of Aβ42 in our selection circuit (FIG. 2B). We used an SP-encoded CZ₆ SICLOPPS library (Z=NDT codon encoded amino acid: R, N, D, C, G, H, I, L, F, S, Y, or V) (˜3×10⁶ total sequences). This codon set was chosen to reduce the theoretical library size and facilitate library cloning. The library (total of ˜5×10⁶ transformants) was cloned on a SP obtained from an additional 250 hours of PACE using a more stringent selection (FIGS. 10A-10D).

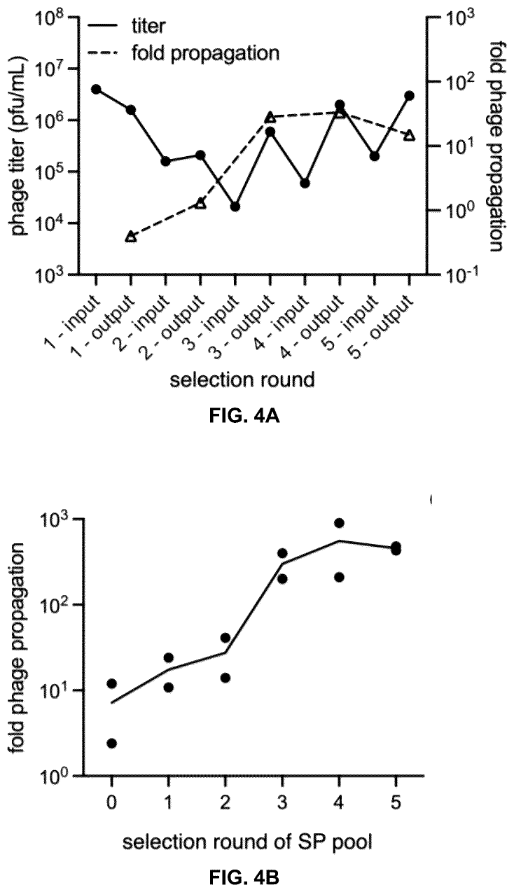

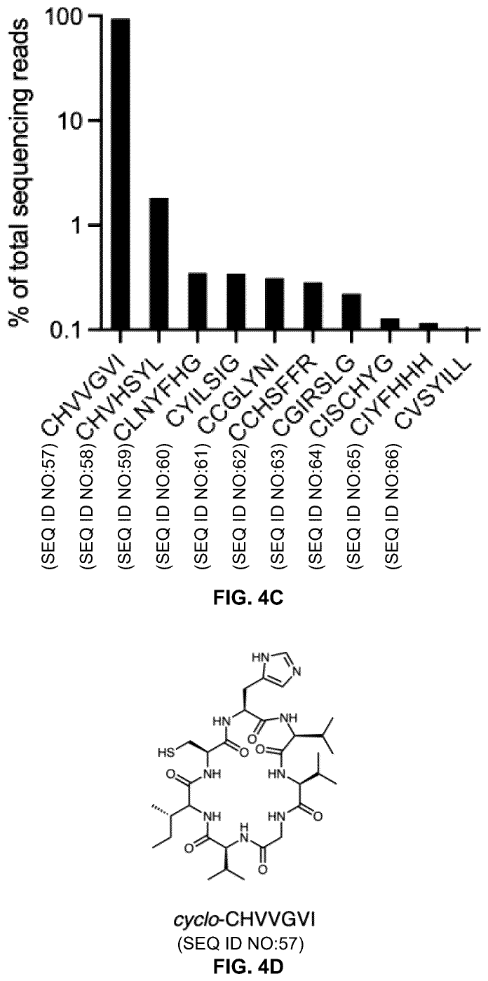

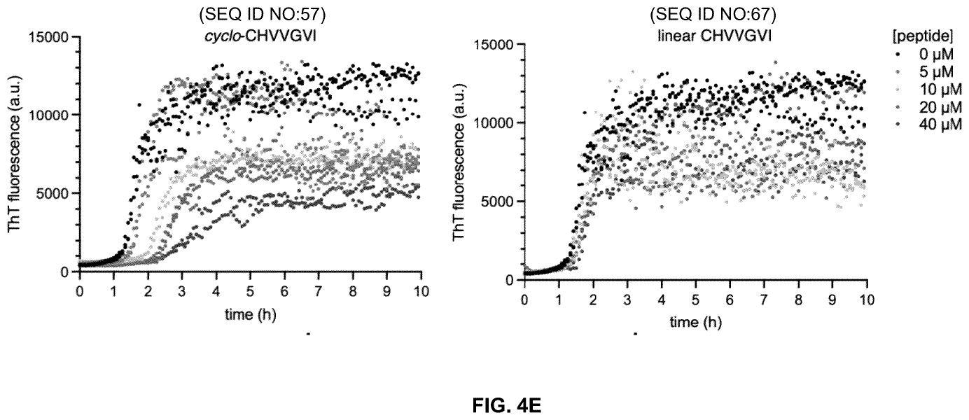

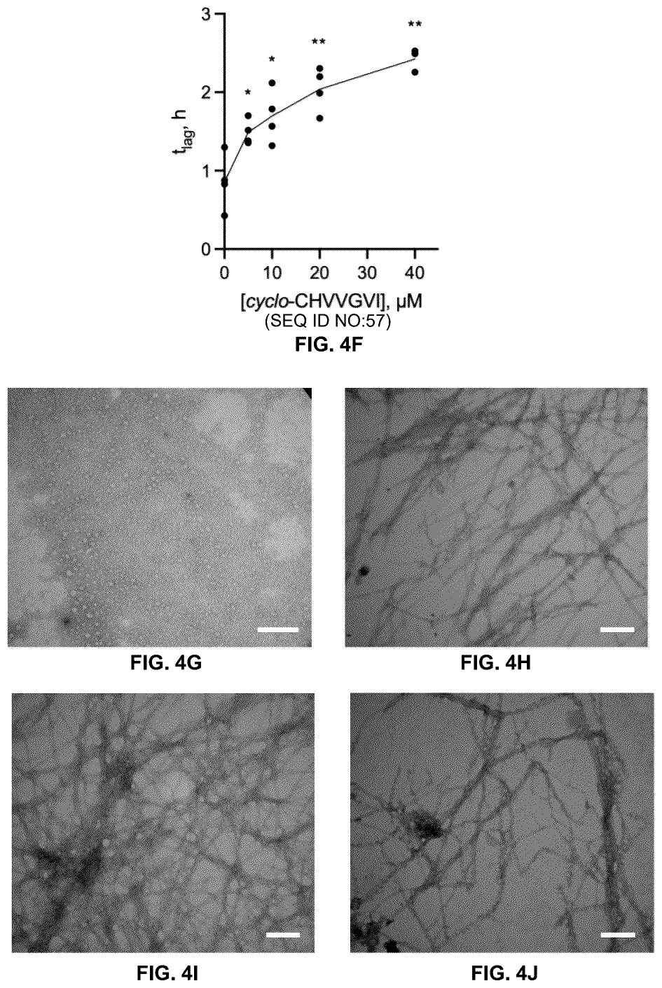

We performed five rounds of selection, diluting the SP pool by ten-fold each round (FIG. 4A). There was a modest increase in SP pool fitness over the course of the selection (˜35-fold). To verify that we selected phage with higher activity, we measured the propagation of the SP from each round on hIAPP selection cells, normalizing input phage titers to 105 pfu/mL to enable head-to-head comparison between phage pools. We observed a ˜50-fold increase in SP pool fitness (FIG. 4B), suggesting enrichment of active cyclic peptides by the selection. Deep sequencing of the SP pool from round 5 revealed that a single sequence (CHWGVI (SEQ ID NO:57)) accounted for ˜95% of sequencing reads (FIGS. 4C and 4D), with the second most enriched sequence, CHVHSYL (SEQ ID NO:58), exhibiting some similarity to the top hit. Aside from these two sequences, we did not observe any clear trends in the remaining enriched cyclic peptides.

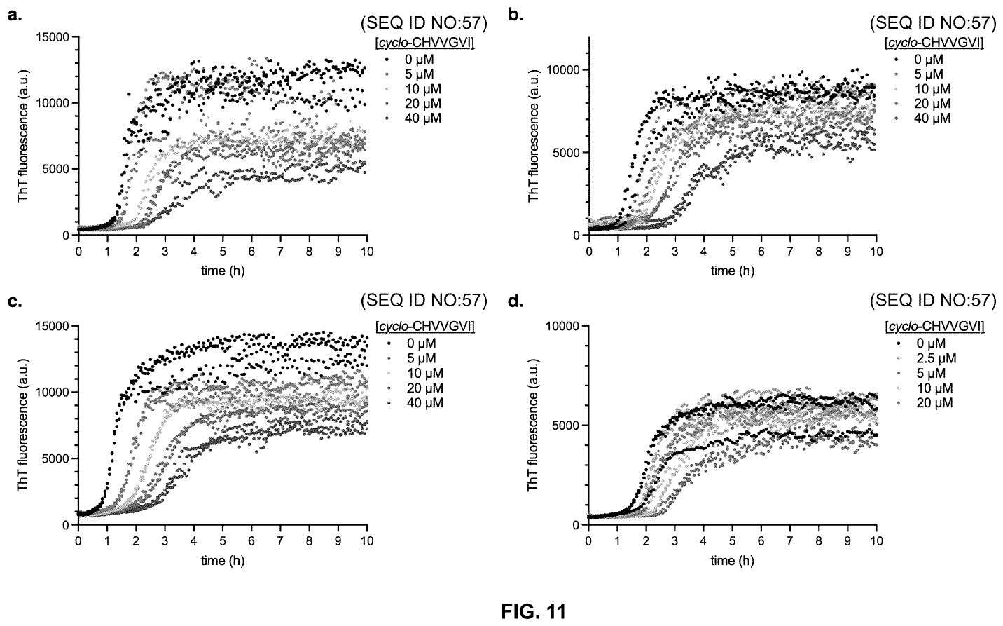

We synthesized both cyclo-CHWGVI (SEQ ID NO:57) (FIG. 4D) and linear CHVVGVI (SEQ ID NO:67) and measured their effects on hIAPP aggregation in vitro. cyclo-CHWGVI (SEQ ID NO:57) inhibited hIAPP aggregation in thioflavin T (ThT) fluorescence assays (FIG. 4E and FIG. 11), delaying the aggregation lag time (t_{lag}) in a dose-dependent fashion (FIG. 4F). In contrast, linear CHWGVI (SEQ ID NO:67) showed greatly reduced ability to inhibit hIAPP (FIG. 4E), suggesting that cyclization is important for activity.

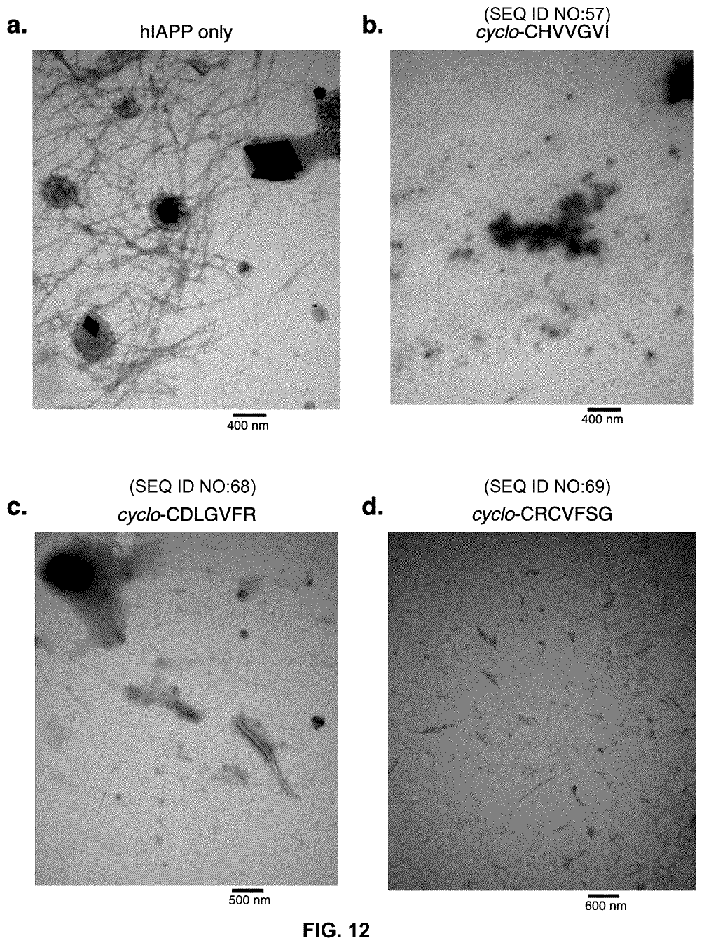

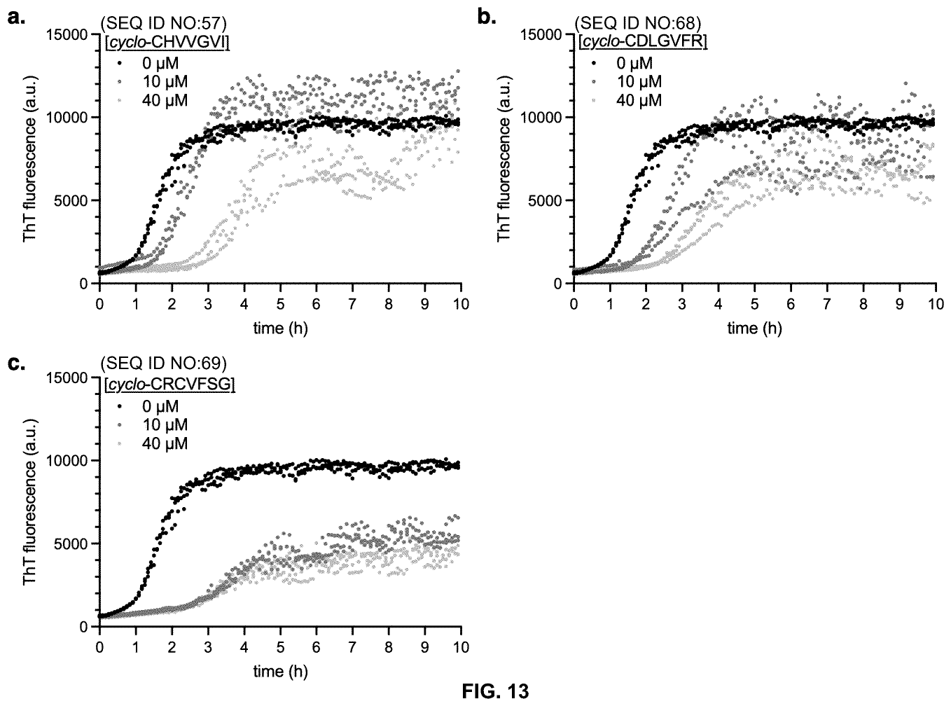

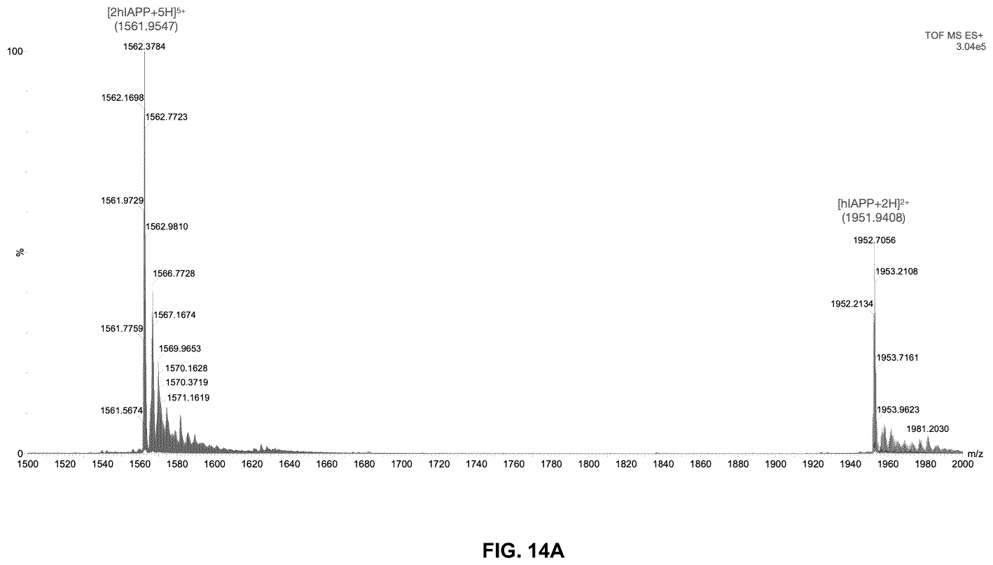

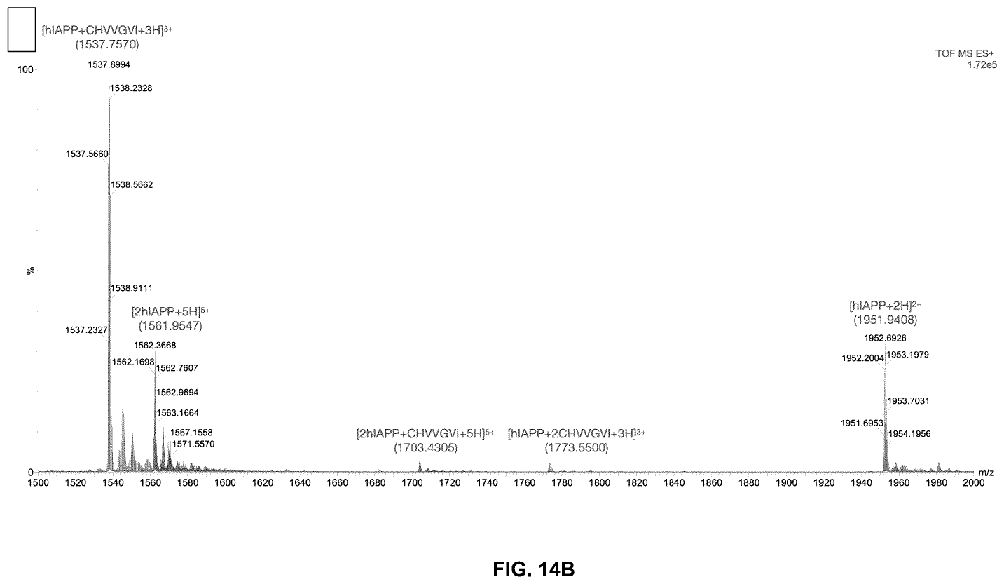

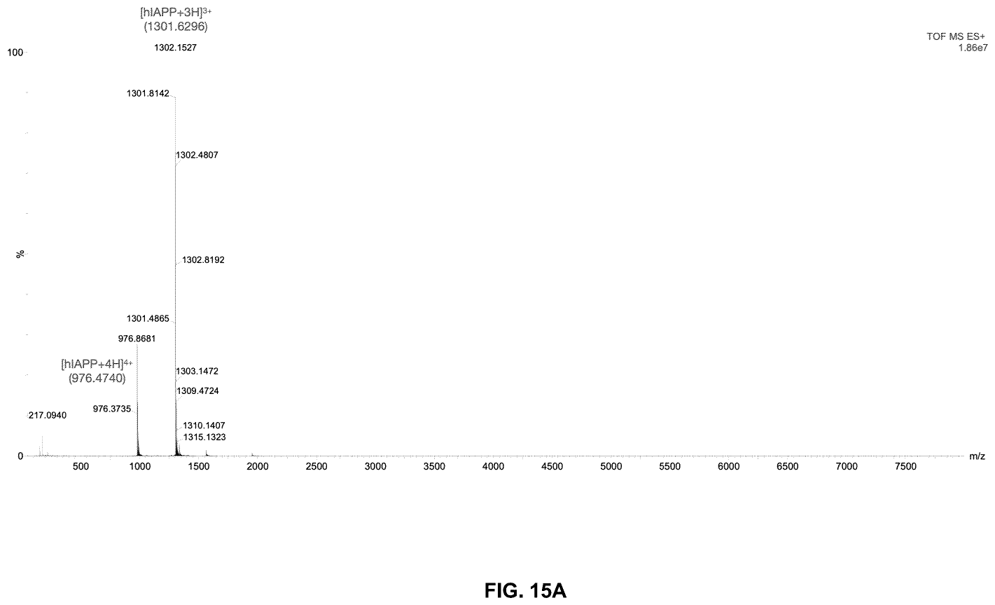

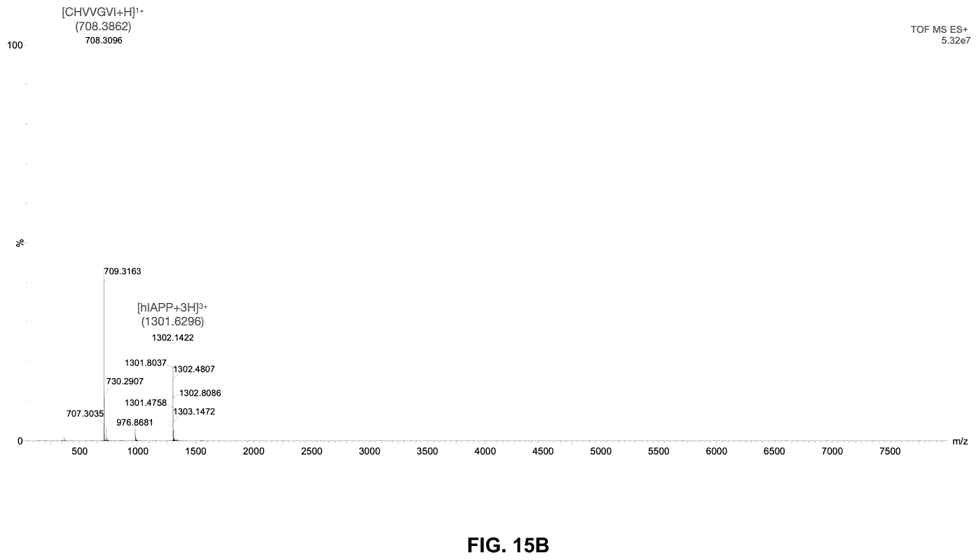

We further characterized the interaction of cyclo-CHVVGVI (SEQ ID NO:57) with hIAPP. TEM analysis corroborated the ability of cyclo-CHVVGVI (SEQ ID NO:57) to delay hIAPP aggregation onset, showing that no fibrils are observed when hIAPP is incubated with cyclo-CHVVGVI (SEQ ID NO:57) for 2 h (FIG. 4G and FIG. 12). In contrast, hIAPP produces numerous fibrils when incubated alone under the same conditions (FIG. 4H and FIG. 12). No gross differences in morphology were observed between fibrils formed by hIAPP alone or with cyclic peptide after incubation at longer times when all hIAPP should be aggregated (FIGS. 41 and 4J), suggesting that cyclo-CHVVGVI (SEQ ID NO:57) is not being incorporated into hIAPP fibrils or sequestering hIAPP in a different form of aggregate. Finally, analysis of the interaction of cyclo-CHWGVI (SEQ ID NO:57) with hIAPP by native mass spectrometry primarily detected a 1:1 complex between cyclo-CHVVGVI (SEQ ID NO:57) and hIAPP (FIGS. 14A and 14B), suggesting that cyclo-CHVVGVI (SEQ ID NO:57) inhibits hIAPP aggregation through binding to monomeric hIAPP, consistent with the APP-T7n selection mechanism (FIG. 2A).

These results demonstrate that our system can be used to rapidly identify cyclic peptide inhibitors of a different aggregation-prone protein.

### Negative Selection Identifies Selective Aggregation Inhibitors

The top sequences enriched by the hIAPP selection (CHWGVI (SEQ ID NO:57) and CHVHSYL (SEQ ID NO:58)) resembled previously identified Aβ42 aggregation inhibitors²⁰ and sequences from our own selections on Aβ42-T7n. Because the amyloidogenic regions of Aβ42 and hIAPP share sequence similarity,⁴² we wondered if our selection for hIAPP inhibitors discovered peptides that are also active on Aβ42. Indeed, the hIAPP round 5 SP pool exhibited activity on both hIAPP-T7n and Aβ42-T7n (FIG. 5A), suggesting that we had identified promiscuous sequences. However, the SP pools were inactive against a non-amyloidogenic target (an scFv that aggregates in E. coli cytosol). Non-specific hits and false positives are a concern for selection-based cyclic peptide discovery methods.^{14,43} Negative selection can increase selectivity and weed out false positives, but to our knowledge has not been explored in cellular cyclic peptide selections. These results motivated us to develop a negative selection that could quickly exclude cyclic peptides with non-selective activity or act outside of the APP in our selection circuit (for example, binding to and stabilizing T7n).

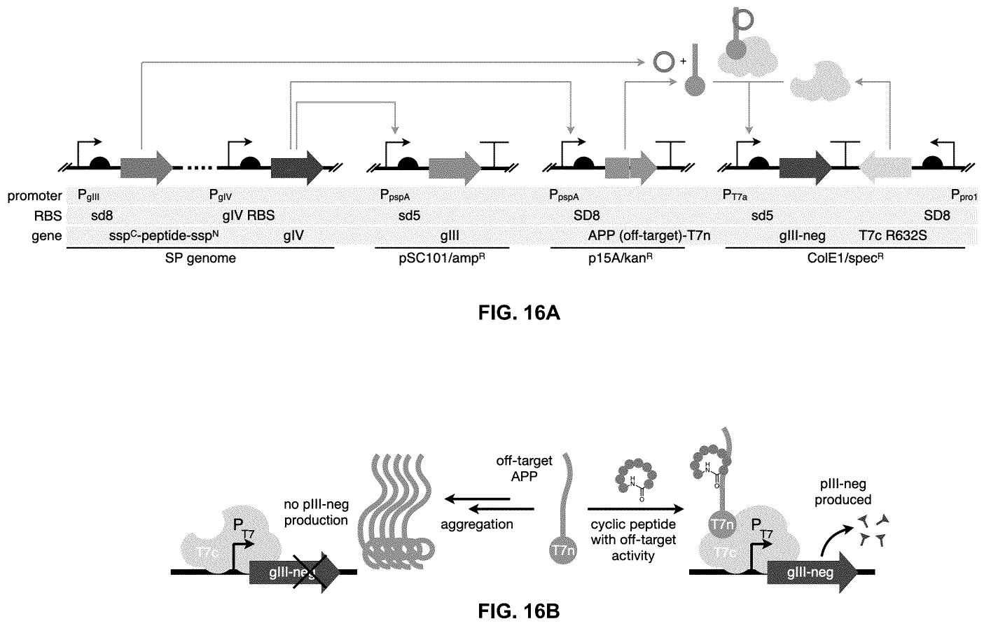

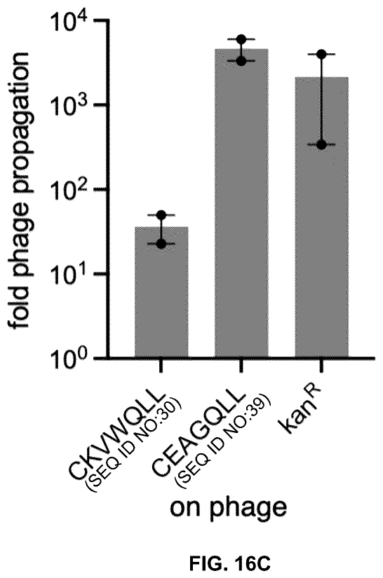

To create a negative selection, we placed gIII-neg, a dominant-negative form of gIII,⁴⁴ under P_{T7} in our selection circuit. Inhibition of a non-target APP (ex. Aβ42) triggers pIII-neg expression and poisons phage proliferation. A second plasmid produces small amounts of pIII from the phage shock promoter (FIG. 16A). All SPs infecting cells encoding this negative selection receive sufficient pIII to propagate. However, SP that carry a promiscuous or false positive cyclic peptide will also produce pIII-neg and generate non-infectious progeny (FIGS. 16B and 16C). We subjected the hIAPP round 5 phage population to four rounds of negative selection, diluting the phage by 100-fold each round (FIG. 5B). The starting fitness of the hIAPP SP pool on the negative selection was low, confirming presence of library members active on Aβ42. However, by the fourth round of negative selection, the SP pool fitness had increased by ˜1000-fold, suggesting removal of promiscuous sequences (FIG. 5B). Comparison of the activity of the pre- and post-negative selection SP pools revealed a ˜1000-fold decrease in Aβ42-T7n aggregation inhibition, while the activity on hIAPP-T7n decreased modestly (<10-fold; FIG. 5C).

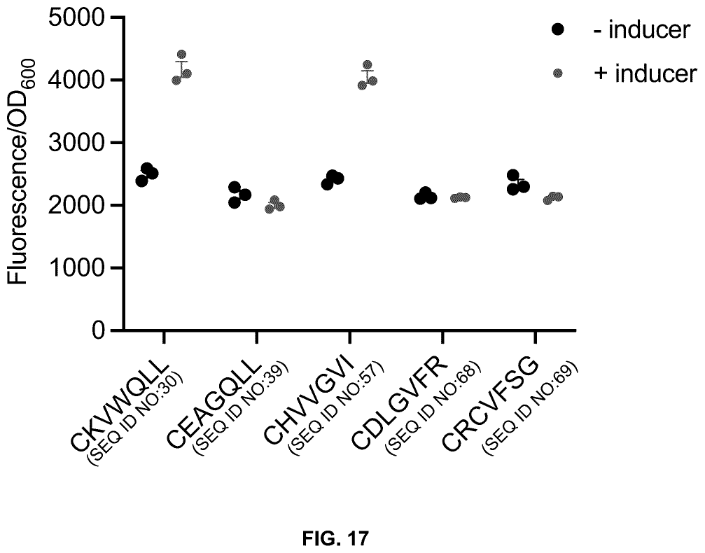

Deep sequencing of the post-negative selection pool (FIG. 5D and Table 1) revealed that CHVVGVI (SEQ ID NO:57) was absent from the top 10 sequences. Instead, >50% of sequencing reads mapped to CDLGVFR (SEQ ID NO:68) and CRCVSFG (SEQ ID NO:69) (FIG. 5D). These sequences were not highly enriched by positive selection (FIG. 4C), illustrating the ability of negative selection to produce large shifts in the cyclic peptide sequence distribution. We compared the activity of clonal SP encoding CHWGVI (SEQ ID NO:57), CDLGVFR (SEQ ID NO:68), or CRCVSFG (SEQ ID NO:69) on selection cells encoding either hIAPP-T7n or Aβ42-T7n (FIG. 5E). While CHWGVI SP propagated robustly on both targets, CRCVSFG SP were more selective, with ˜10-fold higher activity on hIAPP vs. Aβ42 selection cells. SP encoding CDLGVFR (SEQ ID NO:68) were strikingly selective, exhibiting ˜1000-fold greater propagation on hIAPP vs. Aβ42. We observed similar trends with the Aβ42-GFP folding reporter when CHWGVI (SEQ ID NO:57), CDLGVFR (SEQ ID NO:68), and CRCVSFG (SEQ ID NO:69) were expressed from plasmid (FIG. 17), suggesting that selectivity is not an artifact of our phage system.

TABLE 1
Comparison of top 10 cyclic peptide sequences enriched from
positive selection on hIAPP or subsequent negative selection
against Aβ42 as determined by high-throughput sequencing.

| | | Percent | | Percent | Fold |
|---|---|---|---|---|---|
| | | total | | total | enrichment in |
| | Rank | reads | Rank | reads | negative vs. |
| | (positive | (positive | (negative | (negative | positive |
| Sequence | selection) | selection) | selection) | selection) | selection |
| CHVVGVI (SEQ ID NO: 57) | 1 | 94.40 | 20 | 0.29 | 0.003 |
| CHVHSYL (SEQ ID NO: 58) | 2 | 1.82 | 4 | 6.03 | 3.31 |
| CLNYFHG (SEQ ID NO: 59) | 3 | 0.35 | 3 | 7.05 | 20.08 |
| CYILSIG (SEQ ID NO: 60) | 4 | 0.34 | 18 | 0.37 | 1.10 |
| CCGLYNI (SEQ ID NO: 61) | 5 | 0.31 | 31 | 0.15 | 0.48 |
| CCHSFFR (SEQ ID NO: 62) | 6 | 0.29 | 12 | 0.83 | 2.87 |
| CGIRSLG (SEQ ID NO: 63) | 7 | 0.22 | 7 | 4.48 | 20.09 |
| CISCHYG (SEQ ID NO: 64) | 8 | 0.13 | 34 | 0.12 | 0.92 |
| CIYFHHH (SEQ ID NO: 65) | 9 | 0.12 | 8 | 3.33 | 28.44 |
| CVSYILL (SEQ ID NO: 66) | 10 | 0.11 | 16 | 0.45 | 4.21 |
| CDLGVFR (SEQ ID NO: 68) | 14 | 0.08 | 1 | 31.22 | 410.79 |
| CRCVFSG (SEQ ID NO: 69) | 20 | 0.04 | 2 | 24.55 | 701.46 |
| CFNLVVD (SEQ ID NO: 72) | 15 | 0.08 | 5 | 4.85 | 64.65 |
| CFFRGSD (SEQ ID NO: 73) | 18 | 0.06 | 6 | 4.73 | 81.53 |
| CNRLDVS (SEQ ID NO: 76) | 26 | 0.02 | 9 | 1.84 | 83.68 |
| CGLGHGN (SEQ ID NO: 77) | 53 | 0.009 | 10 | 1.12 | 124.22 |

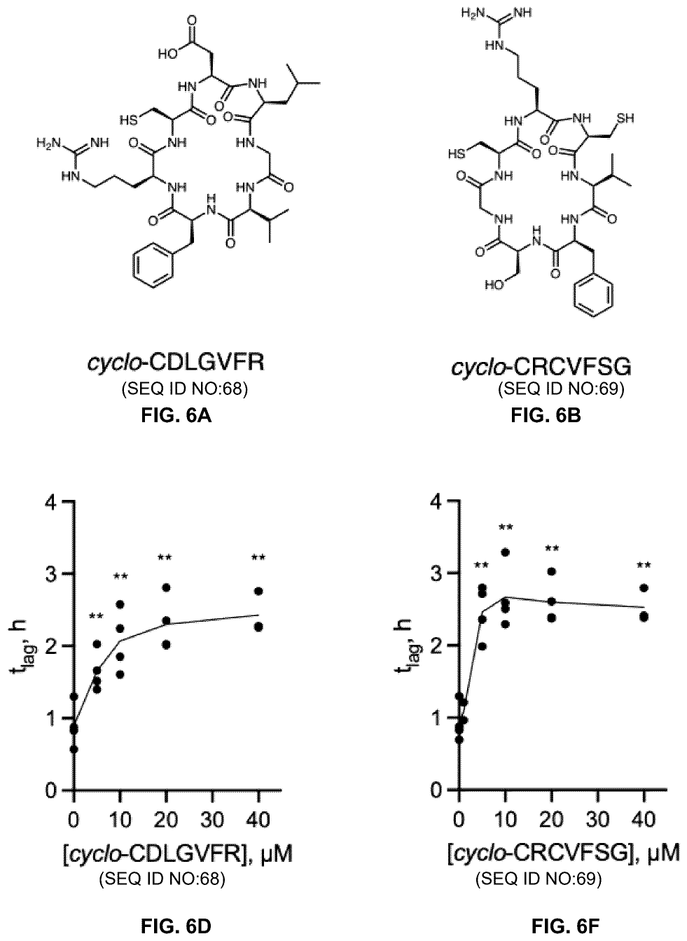

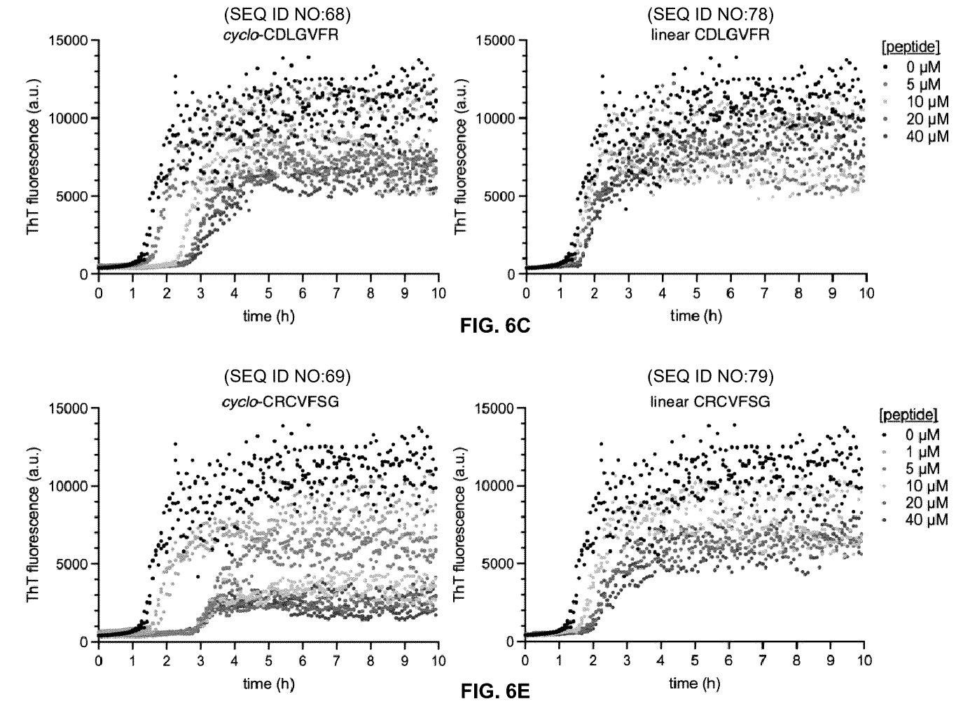

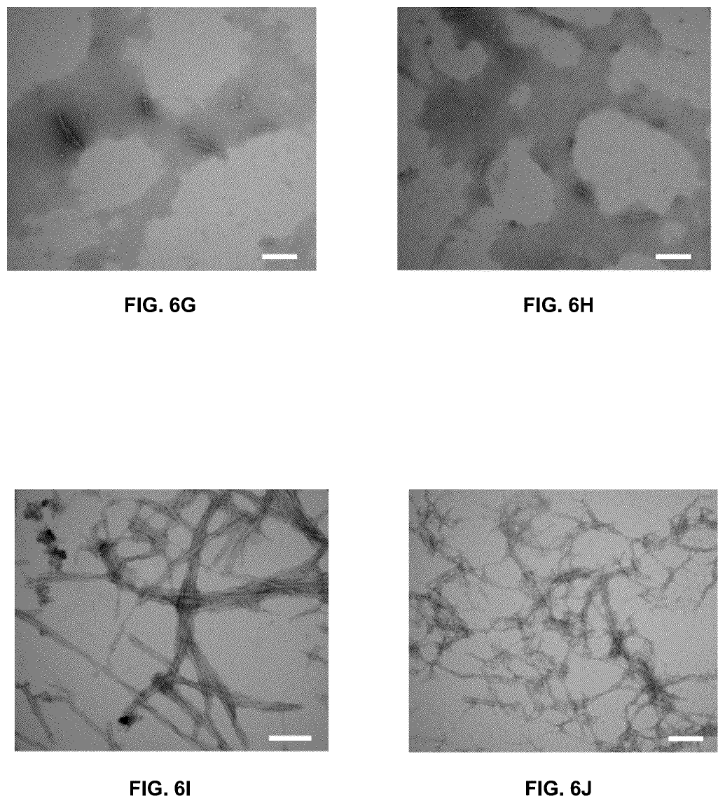

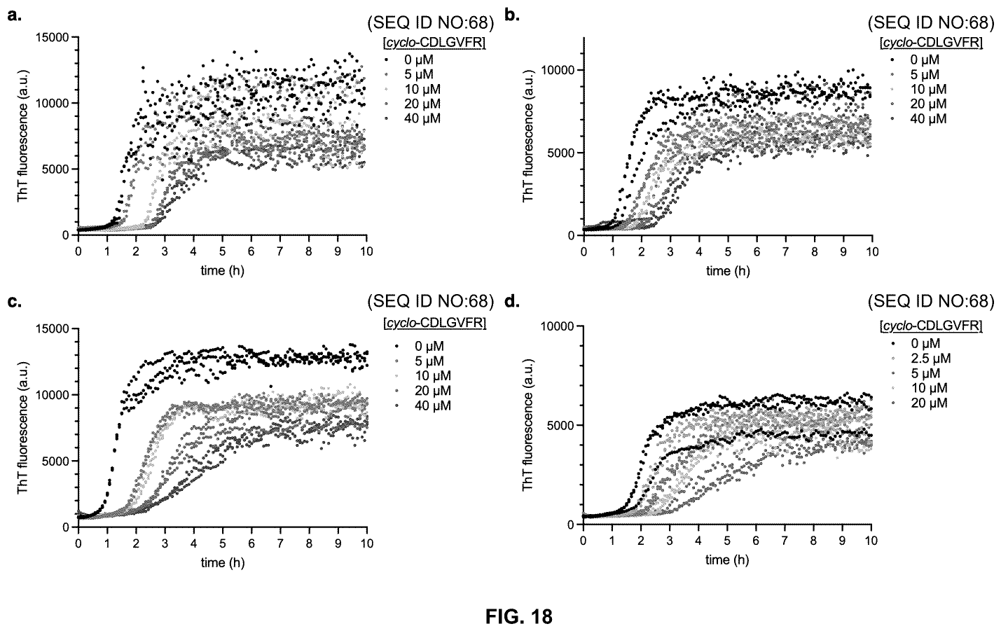

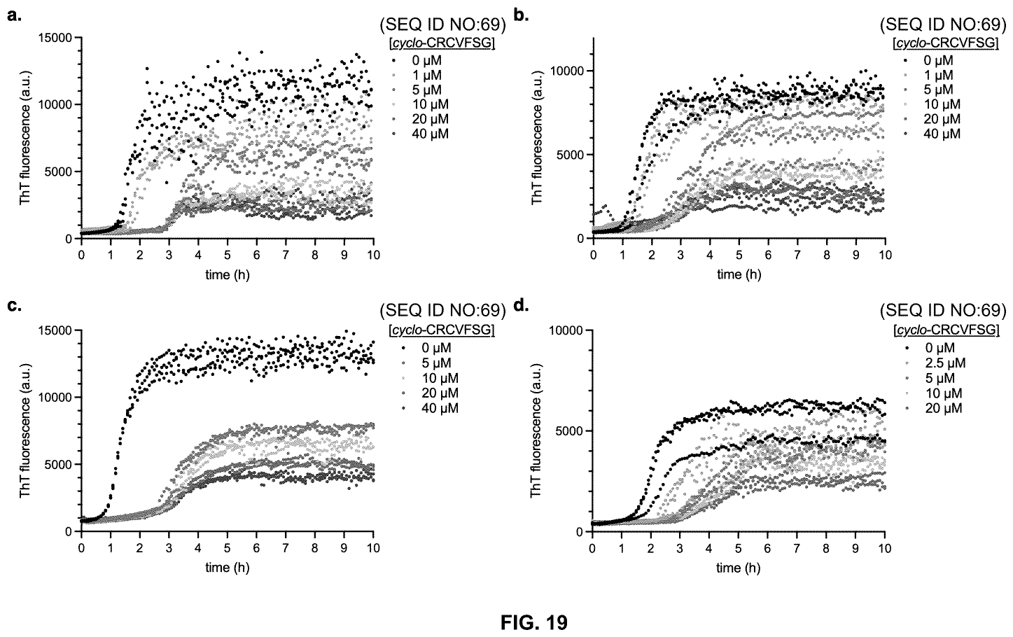

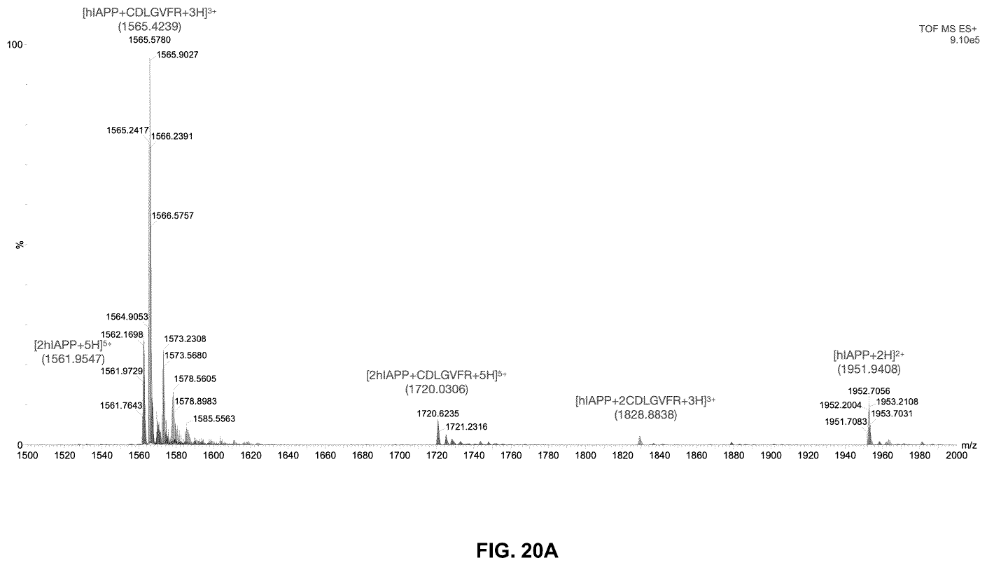

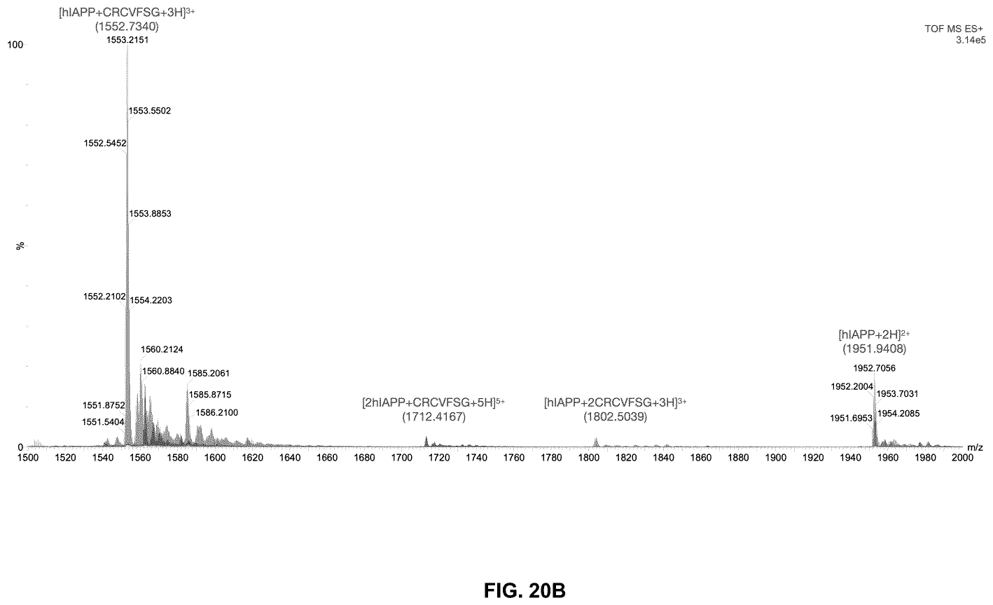

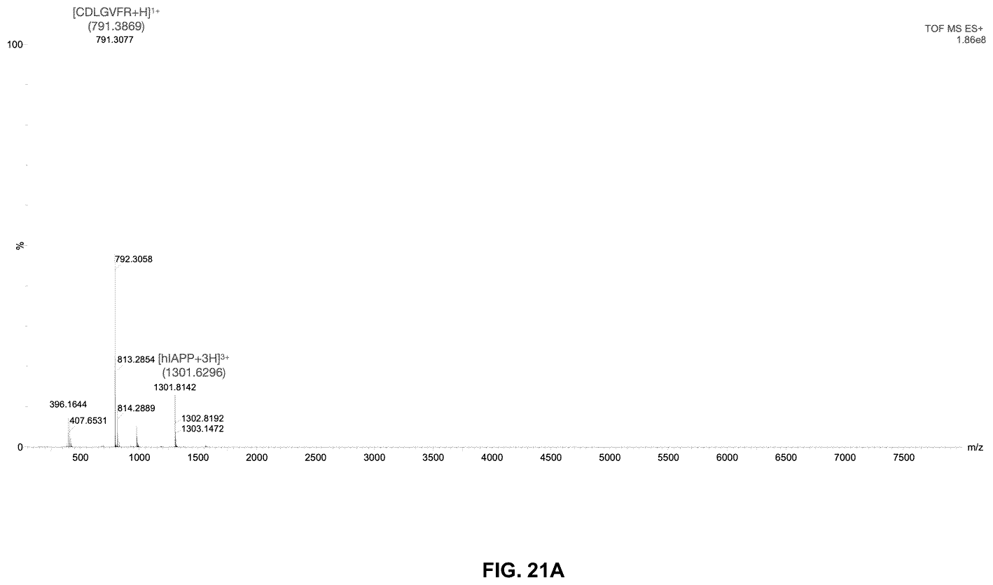

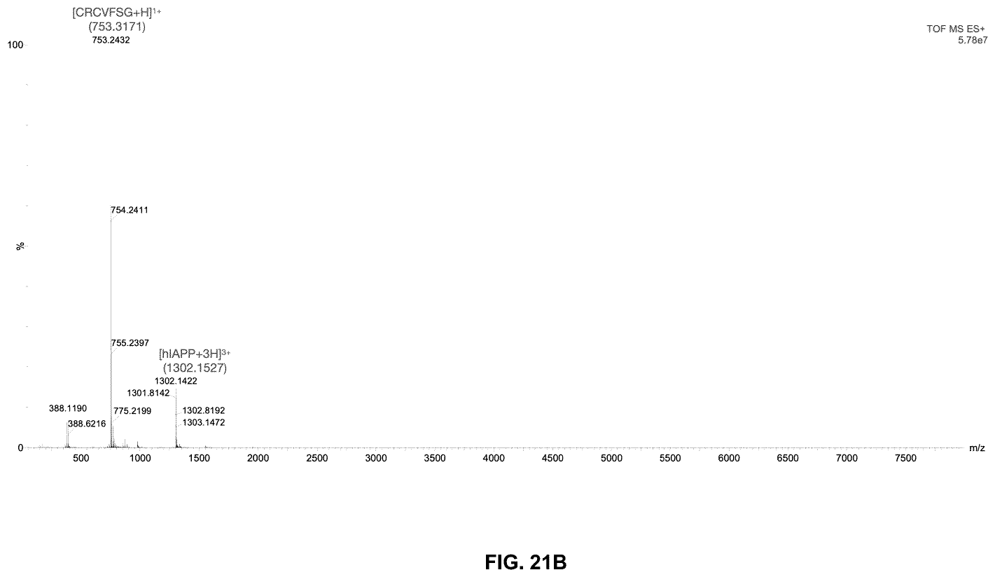

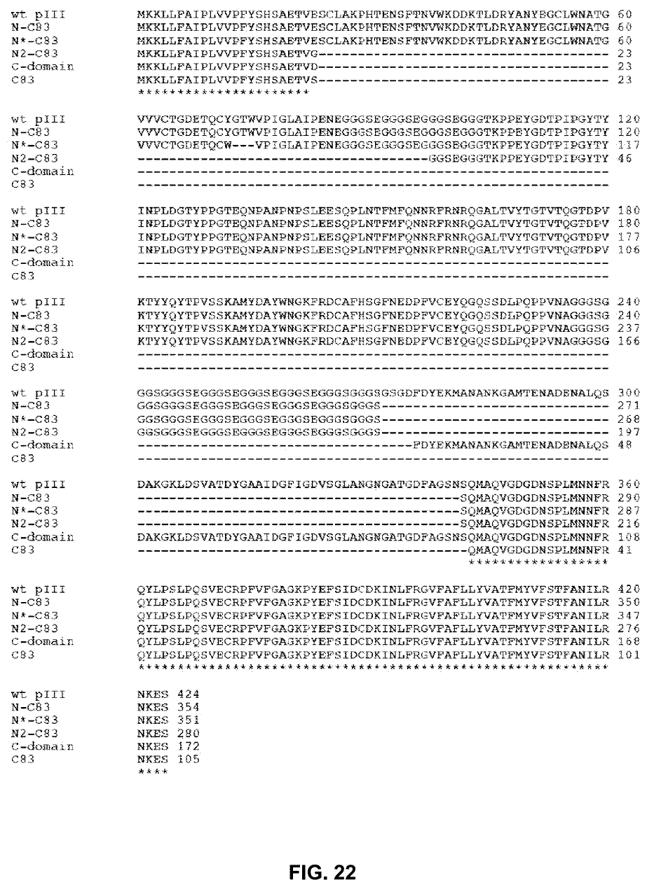

We examined the ability of chemically synthesized cyclo-CDLGVFR (SEQ ID NO:68) and cyclo-CRCVSFG (SEQ ID NO:69) (FIGS. 6A and 6B) to inhibit in vitro hNAPP aggregation. Both cyclic peptides inhibited hNAPP fibrillation and extended t_{lag} (FIGS. 6C-6F and FIGS. 18 and 19). In contrast, linear CDLGVFR (SEQ ID NO:78) was completely inactive (FIG. 6C), while linear CRCVSFG (SEQ ID NO:79) moderately inhibited hNAPP aggregation, but with reduced activity compared to cyclo-CRCVFSG (SEQ ID NO:69) (FIG. 6E). Like cyclo-CHWGVI (SEQ ID NO:57), cyclo-CDLGVFR (SEQ ID NO:68) and cyclo-CRCVSFG (SEQ ID NO:69) exhibited similar activity to MCIP-2a at lower concentrations, but were less active than MCIP-2a at higher peptide:hIAPP stoichiometries. TEM analysis confirmed that both cyclic peptides delay hNAPP aggregation (FIGS. 6G and 6H and FIG. 12) and do not change the overall appearance of hNAPP fibrils (FIGS. 61 and 6J). Additionally, we observed 1:1 complexes of both cyclo-CDLGVFR (SEQ ID NO:68) and cyclo-CRCVSFG (SEQ ID NO:69) with hNAPP by native mass spectrometry (FIGS. 20A and 20B), suggesting that, like cyclo-CHWGVI (SEQ ID NO:57), these cyclic peptides are primarily hNAPP monomer binders.

Together, these results confirmed that our negative selection had purged promiscuous sequences to successfully uncover active and selective cyclic peptide inhibitors of hNAPP aggregation.

### Discussion

We developed a new platform for identifying cyclic peptide protein aggregation inhibitors that leverages elements of PACE to increase the speed and convenience of selection. This method does not require specialized equipment (such as a cell sorter) and, aside from the initial generation of the SP library, eliminates the need for additional cloning or transformation steps. As a demonstration of the platform's utility, we applied it to identify new cyclic peptide inhibitors of hIAPP aggregation in less than a week. In addition, the observation that enriched inhibitors also exhibited activity on a different aggregation-prone protein, Aβ42, prompted us to create a negative selection strategy to remove promiscuous sequences. We used this negative selection to identify cyclic peptides that selectively inhibited hIAPP. Off-target activity or false positives can plague selections and high-throughput screens of chemical libraries for active compounds, resulting in extra time and labor expended on isolating desired hits. Thus, negative selections such as the one used here can improve hit quality by purging undesired library members and streamline the isolation of desirable cyclic peptide sequences.

To our knowledge, the cyclic peptide hIAPP inhibitors we report are the first to be identified through a selection-based method. Previously, macrocyclic peptides have been generated by rational design to inhibit hIAPP aggregation through displayed hIAPP sequence mimics^{41,45} or aromatic moieties.^{46,47} Our cyclic peptides bear little sequence resemblance to rationally designed inhibitors, suggesting that unbiased selection can uncover new starting points for inhibitor development. Future efforts may improve the potency of hIAPP inhibitors identified by our platform through developing more stringent selections that require more active cyclic peptides to pass.

An unexpected observation made here is that the SICLOPPS cyclic peptide sequences from this work and other studies²⁰ may not require intein splicing to inhibit target protein aggregation. Because splicing efficiency varies by extein sequence,⁴⁹ it is difficult to predict the ratio of unspliced to spliced product for each cyclic peptide library member. Thus, the species under selection may be an intein-bound or spliced form of the peptide, or both. Our hits are active as chemically synthesized cyclic peptides and exhibit diminished activity when employed in linear form, suggesting that a cyclic conformation is important for activity in our selection for hIAPP inhibitors. However, other targets could behave differently, and moving forward, it may be preferable to switch the Ssp intein in SICLOPPS with with faster splicing homologs to minimize the time cyclic peptide sequences undergoing selection spend in the intein-bound form.^{49,50}

One limitation of our platform is the size of the cyclic peptide libraries employed, which is restricted by cloning efficiencies. This constraint is not unique to our system and applies to other cellular selections.^{13,16} A potential route to increasing library size is to further mutate the SP using host cell-encoded mutagenesis plasmids.³⁹

Although here we exclusively select for protein aggregation inhibitors, in principle our platform could be used to select for any cyclic peptide activity that can be linked to a genetic selection. PACE selections have been developed for a wide range of biologically significant activities, such as protein-protein interaction,^{28,51} protein-DNA interaction,^{35,52,53} and ternary complex formation²⁶ to name a few, and these selections could be adapted for use in cyclic peptide discovery. Our system may also be able to accommodate other types of genetically encoded peptide libraries.^{16,18} Thus, we anticipate that our work will provide a versatile and accessible new option for cyclic peptide discovery.

### Materials and Methods

### General Methods

Antibiotics (Gold Biotechnology) were used at the following working concentrations: ampicillin, 50 μg/L; spectinomycin, 100 μg/mL; chloramphenicol, 25 μg/mL; kanamycin, 50 μg/mL; tetracycline, 10 μg/mL; streptomycin, 50 μg/mL. HyClone water (GE Healthcare Life Sciences) was used for PCR reactions and cloning. For all other experiments, water was purified using a MilliQ purification system (Millipore). Phusion U Hot Start DNA polymerase (Thermo Fisher Scientific) or Q5 polymerase (New England Biolabs) were used for PCRs. A full list of plasmids used in this work is given in Table 2. Key primers are provided in Table 3.

TABLE 2
Plasmids and selection phages used in this work.

| Plasmid | | | ORF1 | | | ORF2 | | |
|---|---|---|---|---|---|---|---|---|
| Name | Resistance | Origin | promoter | RBS | gene(s) | promoter | RBS | gene |
| MP6³⁹ | chlor | cloDF13 | P_{BAD} | native | dnaQ926, dam, seqA, | | | |
| | | | | dnaQ | emrR, PBS2 UGI, | | | |
| | | | | RBS | pmCDA1 | | | |
| pLY009 | spec | ColE1 | Pₚₛₚ | SD8⁵⁶ | Aβ42-T7n | | | |
| pTW006ap1a³³ | amp | SC101 | P_{T7a} | SD8 | gIII (recoded), luxAB | Pₚᵣₒ₁⁵⁷ | SD8 | T7c |
| pTW357b | spec | ColE1 | P_{T7a} | sd5⁵⁶ | gIII (recoded) | Pₚᵣₒ₁⁵⁷ | SD8 | T7c |
| | | | | | | | | R632S |
| pTW357d5 | spec | ColE1 | P_{T7a} | sd5 | gIII (recoded) | Pₚᵣₒ₅⁵⁷ | SD8 | T7c |
| | | | | | | | | R632S/ |
| | | | | | | | | Q649S |
| pTW357bN | spec | ColE1 | P_{T7a} | sd5 | gIII-neg | Pₚᵣₒ₁⁵⁷ | SD8 | T7c |
| | | | | | | | | R632S |
| pTW358b | kan | p15A | Pₚₛₚ | SD8 | Aβ42-T7n | | | |
| pTW358c | kan | p15A | Pₚₛₚ | SD8 | Aβ42 F19S/L34P-T7n | | | |
| pTW358f | kan | p15A | Pₚₛₚ | SD8 | hIAPP-T7n | | | |
| pTW358d | kan | p15A | Pₚₛₚ | SD8 | 6xHis-scFv-C11-T7n | | | |
| pLY034b | amp | pSC101 | Pₚₛₚ | sd5 | gIII (recoded) | | | |
| pBL066c | spec | ColE1 | Pₜₑₜ | SD8 | Aβ42-GFP | | | |
| pLY002tac | kan | p15A | P_{tac} | SD8 | SICLOPPS precursor for | | | |
| | | | | | cyclo-CKVWQLL (SEQ ID | | | |
| | | | | | NO: 30) (parent) | | | |
| pLY002tacB | kan | p15A | P_{tac} | SD8 | SICLOPPS precursor for | | | |
| | | | | | cyclo-CEAGQLL (SEQ ID | | | |
| | | | | | NO: 39) (parent) | | | |
| pLY002tacD | kan | p15A | P_{tac} | SD8 | splicing-deficient | | | |
| | | | | | SICLOPPS precursor for | | | |
| | | | | | cyclo-CKVWQLL (SEQ ID | | | |
| | | | | | NO: 30) (parent) | | | |
| pLY063a | kan | p15A | P_{tac} | SD8 | SICLOPPS precursor for | | | |
| | | | | | cyclo-CKVWQLL (SEQ ID | | | |
| | | | | | NO: 30) (PACE 1 clone 2) | | | |
| pLY064D | kan | p15A | P_{tac} | SD8 | splicing-deficient | | | |
| | | | | | SICLOPPS precursor for | | | |
| | | | | | cyclo-CKVWQLL (SEQ ID | | | |
| | | | | | NO: 30) (PACE 1 clone 2) | | | |
| pLY063B | kan | p15A | P_{tac} | SD8 | SICLOPPS precursor for | | | |
| | | | | | cyclo-CEAGQLL (SEQ ID | | | |
| | | | | | NO: 39) (PACE 1 clone 2) | | | |
| pLY090a | kan | p15A | P_{tac} | SD8 | SICLOPPS precursor for | | | |
| | | | | | cyclo-CRVWCAR (SEQ | | | |
| | | | | | ID NO: 43) (PACE 1 clone | | | |
| | | | | | 2) | | | |
| pLY090b | kan | p15A | P_{tac} | SD8 | SICLOPPS precursor for | | | |
| | | | | | cyclo-CRVYQVL (SEQ ID | | | |
| | | | | | NO: 44) (PACE 1 clone 2) | | | |
| pLY071 | kan | p15A | P_{tac} | SD8 | SICLOPPS precursor for | | | |
| | | | | | cyclo-CKVWQLL (SEQ ID | | | |
| | | | | | NO: 30) (PACE 2 clone 1) | | | |
| pLY071B | kan | p15A | P_{tac} | SD8 | SICLOPPS precursor for | | | |
| | | | | | cyclo-CEAGQLL (SEQ ID | | | |
| | | | | | NO: 39) (PACE 2 clone 1) | | | |
| pLY071D | kan | p15A | P_{tac} | SD8 | splicing-deficient | | | |
| | | | | | SICLOPPS precursor for | | | |
| | | | | | cyclo-CKVWQLL (SEQ ID | | | |
| | | | | | NO: 30) (PACE 2 clone 1) | | | |
| pLY071c | kan | p15A | P_{tac} | SD8 | SICLOPPS precursor for | | | |
| | | | | | cyclo-CHVVGVI (SEQ ID | | | |
| | | | | | NO: 57) (PACE 2 clone 1) | | | |
| pLY071e | kan | p15A | P_{tac} | SD8 | SICLOPPS precursor for | | | |
| | | | | | cyclo-CDLGVFR (SEQ ID | | | |
| | | | | | NO: 68) (PACE 2 clone 1) | | | |
| pLY071f | kan | p15A | P_{tac} | SD8 | SICLOPPS precursor for | | | |
| | | | | | cyclo-CRCVFSG (SEQ ID | | | |
| | | | | | NO: 69) (PACE 2 clone 1) | | | |
| spLY001 | | M13 | P_{gIII} | sd8⁵⁶ | SICLOPPS precursor for | | | |
| | | | | | cyclo-CKVWQLL (SEQ ID | | | |
| | | | | | NO: 30) (parent) | | | |
| spLY001d | | M13 | P_{gIII} | sd8 | splicing-deficient | | | |
| | | | | | SICLOPPS precursor for | | | |
| | | | | | cyclo-CKVWQLL (SEQ ID | | | |
| | | | | | NO: 30) (parent) | | | |
| spLY002 | | M13 | P_{gIII} | sd8 | SICLOPPS precursor for | | | |
| | | | | | cyclo-CVQWLKL (SEQ ID | | | |
| | | | | | NO: 31) (parent) | | | |
| spLY001M7 | | M13 | P_{gIII} | sd8 | SICLOPPS precursor for | | | |
| | | | | | cyclo-CEAGQLL (SEQ ID | | | |
| | | | | | NO: 39) (parent) | | | |
| spLY014 | | M13 | P_{gIII} | sd8 | SICLOPPS precursor for | | | |
| | | | | | cyclo-CKVWQLL (SEQ ID | | | |
| | | | | | NO: 30) (PACE 1 clone 2) | | | |
| spLY014D | | M13 | P_{gIII} | sd8 | splicing-deficient | | | |
| | | | | | SICLOPPS precursor for | | | |
| | | | | | cyclo-CKVWQLL (SEQ ID | | | |
| | | | | | NO: 30) (PACE 1 clone 2) | | | |
| spLY014b | | M13 | P_{gIII} | sd8 | SICLOPPS precursor for | | | |
| | | | | | cyclo-CEAGQLL (SEQ ID | | | |
| | | | | | NO: 39) (PACE 1 clone 2) | | | |
| spLY014c | | M13 | P_{gIII} | sd8 | SICLOPPS precursor for | | | |
| | | | | | cyclo-CVQWLKL (SEQ ID | | | |
| | | | | | NO: 31) (PACE 1 clone 2) | | | |
| spLY014d | | M13 | P_{gIII} | sd8 | SICLOPPS precursor for | | | |
| | | | | | cyclo-CEVWQLL (SEQ ID | | | |
| | | | | | NO: 33) (PACE 1 clone 2) | | | |
| spLY014e | | M13 | P_{gIII} | sd8 | SICLOPPS precursor for | | | |
| | | | | | cyclo-CKAWQLL (SEQ ID | | | |
| | | | | | NO: 34) (PACE 1 clone 2) | | | |
| spLY014f | | M13 | P_{gIII} | sd8 | SICLOPPS precursor for | | | |
| | | | | | cyclo-CKVGQLL (SEQ ID | | | |
| | | | | | NO: 35) (PACE 1 clone 2) | | | |
| spLY014g | | M13 | P_{gIII} | sd8 | SICLOPPS precursor for | | | |
| | | | | | cyclo-CEAWQLL (SEQ ID | | | |
| | | | | | NO: 36) (PACE 1 clone 2) | | | |
| spLY014h | | M13 | P_{gIII} | sd8 | SICLOPPS precursor for | | | |
| | | | | | cyclo-CEVGQLL (SEQ ID | | | |
| | | | | | NO: 37) (PACE 1 clone 2) | | | |
| spLY014i | | M13 | P_{gIII} | sd8 | SICLOPPS precursor for | | | |
| | | | | | cyclo-CKAGQLL (SEQ ID | | | |
| | | | | | NO: 38) (PACE 1 clone 2) | | | |
| spLY032 | | M13 | P_{gIII} | sd8 | SICLOPPS precursor for | | | |
| | | | | | cyclo-CRVWCAR (SEQ | | | |
| | | | | | ID NO: 43) | | | |
| spLY033 | | M13 | P_{gIII} | sd8 | SICLOPPS precursor for | | | |
| | | | | | cyclo-CRVYQVL (SEQ ID | | | |
| | | | | | NO: 44) | | | |
| spLY032a | | M13 | P_{gIII} | sd8 | SICLOPPS precursor for | | | |
| | | | | | cyclo-ARVWCAR (SEQ ID | | | |
| | | | | | NO: 53) | | | |
| spLY032b | | M13 | P_{gIII} | sd8 | SICLOPPS precursor for | | | |
| | | | | | cyclo-CAVWCAR (SEQ ID | | | |
| | | | | | NO: 54) | | | |
| spLY032c | | M13 | P_{gIII} | sd8 | SICLOPPS precursor for | | | |
| | | | | | cyclo-CRAWCAR (SEQ ID | | | |
| | | | | | NO: 55) | | | |
| spLY032d | | M13 | P_{gIII} | sd8 | SICLOPPS precursor for | | | |
| | | | | | cyclo-CRVACAR (SEQ ID | | | |
| | | | | | NO: 56) | | | |
| spLY006 | | M13 | P_{gIII} | sd8 | SICLOPPS precursor for | | | |
| | | | | | cyclo-CKVWQLL (SEQ ID | | | |
| | | | | | NO: 30) (PACE 2 clone 1 | | | |
| | | | | | with 6xHis tag) | | | |
| spLY006b | | M13 | P_{gIII} | sd8 | SICLOPPS precursor for | | | |
| | | | | | cyclo-CEAGQLL (SEQ ID | | | |
| | | | | | NO: 39) ((PACE 2 clone 1 | | | |
| | | | | | with 6xHis tag) | | | |
| spLY017 | | M13 | P_{gIII} | sd8 | SICLOPPS precursor for | | | |
| | | | | | cyclo-CKVWQLL (SEQ ID | | | |
| | | | | | NO: 30) (PACE 2 clone 1) | | | |
| spLY017b | | M13 | P_{gIII} | sd8 | SICLOPPS precursor for | | | |
| | | | | | cyclo-CEAGQLL (SEQ ID | | | |
| | | | | | NO: 39) (PACE 2 clone 1) | | | |
| spLY017D | | M13 | P_{gIII} | sd8 | splicing-deficient | | | |
| | | | | | SICLOPPS precursor for | | | |
| | | | | | cyclo-CKVWQLL (SEQ ID | | | |
| | | | | | NO: 30) (PACE 2 clone 1) | | | |
| spLY018 | | M13 | P_{gIII} | sd8 | SICLOPPS precursor for | | | |
| | | | | | cyclo-CKVWQLL (SEQ ID | | | |
| | | | | | NO: 30) (PACE 2 clone 2) | | | |
| spLY018b | | M13 | P_{gIII} | sd8 | SICLOPPS precursor for | | | |
| | | | | | cyclo-CEAGQLL (SEQ ID | | | |
| | | | | | NO: 39) (PACE 2 clone 2) | | | |
| spLY018D | | M13 | P_{gIII} | sd8 | splicing-deficient | | | |
| | | | | | SICLOPPS precursor for | | | |
| | | | | | cyclo-CKVWQLL (SEQ ID | | | |
| | | | | | NO: 30) (PACE 2 clone 2) | | | |
| spLY019 | | M13 | P_{gIII} | sd8 | SICLOPPS precursor for | | | |
| | | | | | cyclo-CKVWQLL (SEQ ID | | | |
| | | | | | NO: 30) (PACE 2 clone 3) | | | |
| spLY019b | | M13 | P_{gIII} | sd8 | SICLOPPS precursor for | | | |
| | | | | | cyclo-CEAGQLL (SEQ ID | | | |
| | | | | | NO: 39) (PACE 2 clone 3) | | | |
| spLY019D | | M13 | P_{gIII} | sd8 | splicing-deficient | | | |
| | | | | | SICLOPPS precursor for | | | |
| | | | | | cyclo-CKVWQLL (SEQ ID | | | |
| | | | | | NO: 30) (PACE 2 clone 3) | | | |
| spLY020 | | M13 | P_{gIII} | sd8 | SICLOPPS precursor for | | | |
| | | | | | cyclo-CKVWQLL (SEQ ID | | | |
| | | | | | NO: 30) (PACE 2 clone 4) | | | |
| spLY020b | | M13 | P_{gIII} | sd8 | SICLOPPS precursor for | | | |
| | | | | | cyclo-CEAGQLL (SEQ ID | | | |
| | | | | | NO: 39) (PACE 2 clone 4) | | | |
| spLY020D | | M13 | P_{gIII} | sd8 | splicing-deficient | | | |
| | | | | | SICLOPPS precursor for | | | |
| | | | | | cyclo-CKVWQLL (SEQ ID | | | |
| | | | | | NO: 30) (PACE 2 clone 4) | | | |
| spLY021 | | M13 | PgIII | sd8 | SICLOPPS precursor for | | | |
| | | | | | cyclo-CHVVGVI (SEQ ID | | | |
| | | | | | NO: 57) | | | |
| spLY026a | | M13 | PgIII | sd8 | SICLOPPS precursor for | | | |
| | | | | | cyclo-AHVVGVI (SEQ ID | | | |
| | | | | | NO: 80) | | | |
| spLY026b | | M13 | PgIII | sd8 | SICLOPPS precursor for | | | |
| | | | | | cyclo-CAVVGVI (SEQ ID | | | |
| | | | | | NO: 81) | | | |
| spLY026c | | M13 | PgIII | sd8 | SICLOPPS precursor for | | | |
| | | | | | cyclo-CHAVGVI (SEQ ID | | | |
| | | | | | NO: 82) | | | |
| spLY026d | | M13 | PgIII | sd8 | SICLOPPS precursor for | | | |
| | | | | | cyclo-CHVAGVI (SEQ ID | | | |
| | | | | | NO: 83) | | | |
| spLY026e | | M13 | PgIII | sd8 | SICLOPPS precursor for | | | |
| | | | | | cyclo-CHVVAVI (SEQ ID | | | |
| | | | | | NO: 84) | | | |
| spLY026f | | M13 | PgIII | sd8 | SICLOPPS precursor for | | | |
| | | | | | cyclo-CHVVGAI (SEQ ID | | | |
| | | | | | NO: 85) | | | |
| spLY026g | | M13 | PgIII | sd8 | SICLOPPS precursor for | | | |
| | | | | | cyclo-CHVVGVA (SEQ ID | | | |
| | | | | | NO: 86) | | | |
| spLY026l | | M13 | PgIII | sd8 | SICLOPPS precursor for | | | |
| | | | | | cyclo-CLDCVFG (SEQ ID | | | |
| | | | | | NO: 87) | | | |
| spLY026m | | M13 | PgIII | sd8 | SICLOPPS precursor for | | | |
| | | | | | cyclo-CHLDIGC (SEQ ID | | | |
| | | | | | NO: 88) | | | |
| spLY026n | | M13 | PgIII | sd8 | SICLOPPS precursor for | | | |
| | | | | | cyclo-CLSRRCG (SEQ ID | | | |
| | | | | | NO: 89) | | | |
| spLY022a | | M13 | PgIII | sd8 | SICLOPPS precursor for | | | |
| | | | | | cyclo-CRCVFSG (SEQ ID | | | |
| | | | | | NO: 69) | | | |
| spLY022b | | M13 | PgIII | sd8 | SICLOPPS precursor for | | | |
| | | | | | cyclo-CDLGVFR (SEQ ID | | | |
| | | | | | NO: 68) | | | |

TABLE 3
Key primers used in this work.

| Primer | | |
|---|---|---|
| Name | Sequence (5′ to 3′) | Purpose |
| LY0003 | acagttgUgggcgatcgc (SEQ ID NO: 90) | SP cyclic peptide library |
| | | cloning (reverse primer |
| | | for USER cloning) |
| LY0106 | acaactgUnnknnknnknnknnknnktgtctcag | SP CXXQLX cyclic peptide |
| | tttcggcaccg (SEQ ID NO: 91) | library cloning (forward |
| | | primer for USER cloning) |
| LY0107 | acaactgUndtndtndtndtndtndttgtctcag | SP CZ₆ cyclic peptide |
| | tttcggcaccg (SEQ ID NO: 92) | library cloning (forward |
| | | primer for USER cloning) |
| LY0145 | ggagtgcaaagaatatttgatattgg (SEQ ID | Amplify cyclic peptide |
| | NO: 93) | sequence for HTS |
| LY0146 | caatgggccgtactcaac (SEQ ID NO: 94) | Amplify cyclic peptide |
| | | sequence for HTS |
| LY0147 | acactctttccctacacgacgctcttccgatctg | adapter primer for HTS |
| | gagtgcaaagaatatttgatattgg (SEQ ID | |
| | NO: 95) | |
| LY0148 | gtgactggagttcagacgtgtgctcttccgatct | adapter primer for HTS |
| | caatgggccgtactcaac (SEQ ID NO: 96) | |

Strain S206035 was used for plasmid cloning, amplification, and phage assays. Strain S220835 was used for SP cloning and plaque assays. Plasmids and SPs were cloned by USER assembly or blunt-end ligation. Competent cells were prepared and transformed using the TSS method. Phage propagation and plaque assays were performed as previously described.⁵⁴ Unless otherwise noted, phage propagation assays used an input of 105 phage to infect a 2 mL culture of host cells.

### Phage-Assisted Continuous Evolution

In general, PACE was set up and run as previously described.²⁷

PACE 1. S2060s transformed with pLY009, pTW6ap1a, and MP6 were maintained in a 40 mL chemostat. Lagoons (15 mL each) were infected with evolved spLY001 from PANCE at an initial titer of 4×10⁴ pfu/mL and maintained at a flow rate of 0.5 V/h. Lagoon flow rates were increased to 1 V/h at 19 h, decreased to 0.8 V/h at 66 h, increased back to 1 V/h at 159.5 h, further increased to 1.5 V/h at 257 h, and finally to 2 V/h at 305 h. The experiment ended at 351 h.

PACE 2. S2060s transformed with pTW357d5, pTW358b, and MP6 were maintained in a 40 mL chemostat. Lagoons (15 mL each) were infected with final phage populations collected from PACE 1 at an initial titer of 1×10⁵ pfu/mL and maintained at a flow rate of 0.5 V/h. Lagoon flow rates were increased to 1 V/h at 42.5 h, 1.5 V/h at 117 h, followed by 2 V/h at 160.5 h then 2.5 h at 189 h, and finally 3 V/h at 208 h. The experiment ended at 254.5 h.

### Aβ42-GFP Fluorescence Assay

Single colonies of S2060s transformed with pBL066c and SICLOPPS plasmid were used to make overnight cultures in 2×YT with maintenance antibiotics. The overnight culture was diluted (1:100) into 1 mL DRM on a 96 deep-well plate (VWR, 75870-796) with maintenance antibiotics and IPTG (0, 0.04, 0.2, or 1 mM). The diluted culture was incubated at 37° C. with shaking for 2.5 h, after which aTc (200 ng/mL) was added. 100 μL of the culture was transferred onto a black clear-bottom 96-well plate (VWR, 89131-680) to measure fluorescence signal 2, 3, 5 h after addition of aTc. GFP fluorescence signal (excitation wavelength=485 nm, emission wavelength=535 nm) was then measured using a Tecan M Plex microplate reader.

### Cyclic Peptide Library Cloning

The reverse primer LY0003 and the degenerate forward primers LY0106 (CX₆) or LY0107 (CZ₆) (Table 3) were used to clone the phage-encoded cyclic peptide libraries with spLY006b as the template. The resulting PCR product was purified by PCR clean-up and concentrated to 100 ng/μL. The purified PCR product was assembled by USER assembly. The USER assembled product was purified again by PCR clean-up and diluted to 50 ng/μL. For each aliquot, 250 ng of assembled DNA was transformed into chemicompetent S2208 cells. After heat shock, the aliquots were immediately combined into pre-warmed (37° C.) outgrowth media (2×YT containing 3 mM glucose, 500 μL for each aliquot) and incubated at 37° C. with shaking at 250 rpm for 1 h. After outgrowth, the culture was centrifuged at 8,000 rcf for 2 min and the supernatant containing the phage library was collected. This process resulted in 3×10⁶ and 5×10⁶ independent clonal phage for the CX₆ and CZ₆ libraries, respectively, as measured by plaque assay.

### Cyclic Peptide Library Selection

The phage libraries obtained immediately after cloning were first amplified to introduce degeneracy: mid-log S2208 cells grown in Davis Rich Media (DRM) with maintenance antibiotics at 37° C. with shaking were infected with the phage library and phage propagation allowed to occur for 6 h before the culture was centrifuged at 8,000 rcf for 2 min and the supernatant containing the expanded phage library collected. The expanded phage libraries contained ˜1011 total phage as measured by plaque assay.

Single colonies of host cells transformed with the appropriate selection plasmids were used to make overnight cultures in 2×YT with maintenance antibiotics. The overnight culture was diluted (1:100) into 4-5 mL DRM with maintenance antibiotics and incubated at 37° C. with shaking until the OD₆₀₀ of the culture reached ˜0.5, upon which the expanded phage library was added at a titer of 1×10⁶ pfu/mL. The culture was incubated at 37° C. with shaking overnight (13-18 h). The next day, the culture was centrifuged at 8,000 rcf for 2 min to obtain the supernatant containing phage. The collected phage was diluted into fresh host cell cultures (10 to 1,000-fold) for the next round of selection, while the rest was stored at 4° C. for downstream analysis. This process was repeated until a significant increase in phage propagation was observed compared with the parent library. Both positive and negative selection rounds were performed using this procedure.

### High-Throughput Sequencing and Data Analysis

Sample preparation. Phage stocks collected from the final rounds of library selection were first amplified using LY0145 and LY0146. The PCR product was purified, and adapters added using LY0147 and LY0148. The resulting PCR product was purified and barcoded through a final PCR using index primers to add unique indices for shared Illumina NovaSeq sequencing. The final PCR product was purified, and the concentration measured by Nanodrop. Further sample quality control was performed by the University of Wisconsin-Madison Biotechnology Center (UWBC).

High-throughput sequencing and data analysis. High-throughput sequencing was performed by UWBC using a shared sequencing service on the NovaSeq 6000 platform. Sequencing reads were demultiplexed by UWBC and analyzed using custom-written Python scripts.

### Solid-Phase Peptide Synthesis

General procedure for solid-phase peptide synthesis (SPPS). (see herein for abbreviations) Dawson Dbz AM resin (Sigma-Aldrich) (for head-to-tail cyclized peptides) or Wang resin (CEM) (for linear peptides) was added to an empty SPE cartridge with pre-inserted frit (Agilent, 6 mL capacity). The resin was suspended in 2 mL DCM with shaking and allowed to swell for 40 min at RT. DCM was drained and the resin washed 3 times with 2 mL DCM, followed by 3 times with 2 mL DMF. The resin was deprotected by adding freshly prepared 20% piperidine in DMF and shaking for 30 min, followed by three washes with 2 mL DMF. Couplings were performed with 4 eq. Fmoc-protected L-amino acid, 3.9 eq. HATU, and 8 eq. DIPEA in 2 mL DMF for 1 h with agitation. After coupling, the resin was washed with 2 mL DMF three times. The deprotection and coupling steps were repeated until the peptide was completed. Couplings for Fmoc-L-Arg(Pbf)-OH was repeated once. For head-to-tail cyclic peptides, Boc-L-Cys(Trt)-OH was used as the final amino acid. Upon completion, the resin was washed 3 times with 2 mL DMF and 3 times with 2 mL DCM. Note: the resin was dried by N₂ flow after every washing step in this procedure.

Dbz linker activation. For head-to-tail cyclized peptides, 4-nitrophenyl chloroformate (4 eq) was dissolved in 2 mL DCM and added to the resin. After shaking for 30 min, the solution was drained, and the resin washed 3× with 2 mL DCM. The 4-nitrophenyl chloroformate addition and washing steps were repeated once. The resin was then washed 3× with 2 mL DMF. A solution of 110 μL DIPEA in 2 mL DMF was added and the resin agitated for 10 min, followed by draining the solution. This step was repeated twice. The resin was then washed 3× with 2 mL DMF, 3× with 2 mL DCM, and 3× with 2 mL diethyl ether, then allowed to dry under a flow of N₂. Note: the resin was dried by N₂ flow after every washing step in this procedure.

Deprotection and cleavage. The resin was transferred to a 15 mL conical tube, then treated with cleavage solution (2 mL 90% TFA, 5% DCM, 2.5% H₂O, and 2.5% TIPS) for 2 h with agitation. The solution was filtered into a 50 mL conical tube through an empty syringe plugged with cotton. The remaining resin was washed with another 2 mL cleavage solution and filtered combined into the 50 mL conical in the same manner. 40 mL diethyl ether was added into the conical, which was then incubated at −20° C. for 1 h to facilitate precipitation of peptide product. After precipitation, the solution was centrifuged at 3,000 rpm for 10 min at 4° C. The supernatant was decanted, and the precipitate was dried under N₂ flow to evaporate remaining volatiles. The dried precipitate was then dissolved in 5 mL 20% aq. MeCN, frozen on dry ice for 1 h, and lyophilized.

Linear peptide purification. Lyophilized linear peptide was dissolved in 2 mL 20% aq. MeCN. The solution was passed through a 0.22 μm syringe filter into a fresh 15 mL conical tube. Another 2 mL 20% aq. MeCN was used to wash the old conical and filtered into the new conical tube. All 4 mL of peptide-containing solution was purified by HPLC (Shimadzu, CBM-20A, LC-20AP, SPD-20AV, FRC-10A) in a single injection onto a preparative C18 column (Shimadzu, Premier Elite Polar 10 μ 150×30 mm). HPLC conditions: Flow rate, 25 mL/min. Mobile phase A: H₂O containing 0.1% TFA. Mobile phase B: MeCN. A linear gradient of 10% to 50% mobile phase B over 32 min was used to purify the Nbz-containing linear peptide. Fractions were analyzed by MALDI-TOF to confirm mass of the Nbz-containing linear peptide, then combined and lyophilized.

Cyclization of Nbz-containing linear peptides. Lyophilized linear peptide was dissolved in 4 mL cyclization buffer (0.1 M Na₂HPO₄, 6 M guanidinium chloride, and 20% v/v MeCN in H₂O; pH 6.8-7.2) and incubated at 50° C. with rotation for 2-4 h.

Cyclic peptide purification. After cyclization, the solution was directly injected onto a semi-preparative C18 column (Kromasil, Eternity-5-C18 10×250 mm) HPLC conditions: flow rate, 5 mL/min. Mobile phase A: H₂O containing 0.1% TFA. Mobile phase B: MeCN containing 0.1% TFA. A linear gradient of 20% to 45% mobile phase B over 32 min was used to purify the cyclic peptide. Fractions were analyzed with MALDI-TOF to confirm desired mass of the cyclic peptide, then combined and lyophilized. After lyophilization, H₂O was added to dissolve the cyclic peptide to a stock concentration of 1 mM. The prepared 1 mM cyclic peptide stock solution was quickly aliquoted into low-protein-binding Eppendorf tubes, frozen on dry ice for 1 h and lyophilized to dried peptide solid. Peptides were freshly dissolved before use. Additional freeze-thaw cycles were kept to a minimum to prevent degradation of cyclic peptide stocks.

Cyclic peptide characterization. Analytical HPLC was performed using a HPLC system (Shimadzu, DGU-20A5R, LC-20AT, SIL-10AF, SPD-M20A, CTO-20A) equipped with an analytical C18 column (Kromasil, Eternity-5-C18 4.6×250 mm). HPLC conditions: at a flow rate of 1 mL/min. A binary solvent system with 90% mobile phase A (M.Q. H₂O, 0.1% TFA) and 10% mobile phase B (ACN, 0.1% TFA) was used. A linear gradient of mobile phase B from 10% to 95% within 27 min was used to resolve purity of the sample. Mass of the cyclic peptide was confirmed by ESI-EMM conducted by mass spectrometry facilities in the Paul Bender Chemistry Instrumentation Center (UW-Madison Department of Chemistry).

Synthesis of MCIP-2a. MCIP-2a was synthesized following reported procedures⁴¹ with the following modifications. Briefly, the linear peptide was synthesized on Rink amide resin (CEM) using a Liberty Blue HT24 System and cleaved from the resin as described above. Intramolecular disulfide bridge formation was performed by dissolving crude peptide (after cleavage and lyophilization) at 1 mg/mL in aqueous 0.1 M NH₄HCO₃ solution containing 40% DMSO. The reaction was allowed to proceed for 2 hours at room temperature with agitation. The peptide was then purified by RP-HPLC and characterized by ESI-MS as described above. Purified peptide was prepared in small aliquots, lyophilized, and stored at −80° C. until use.

### ThT Fluorescence Assays

Preparation of ThT stock solution. To prepare a stock solution of ThT (2-3 mM), 2-4 mg of ThT was added to 3 mL PBS (pH 6.9) in a 15 mL conical tube. The solution was sonicated for 5 min and the supernatant was filtered through a 0.22 μm syringe filter into a 1.5 mL Eppendorf tube. This was the ThT stock solution to be used in following experiments. 2 mL of a 1:200 diluted ThT solution was prepared by adding 10 μL ThT stock solution into 1990 μL PBS. The absorbance of the diluted ThT solution measured at 412 nm was used to calculate the ThT stock concentration using an extinction coefficient (c) of 31600 M⁻¹ cm⁻¹.

Preparation of hIAPP stocks. A 1 mg portion of amylin trifluoroacetate salt (Bachem) was purchased as lyophilized solid in a vial. The solid was dissolved in pre-chilled 2562 μL 35 mM sodium acetate (pH 5.3) to a stock concentration of 100 μM on ice. Aliquots of hIAPP were prepared by transferring 200 μL of the 100 μM hIAPP stock solution to low-protein-binding Eppendorf tubes on ice. The aliquots were then immediately snap frozen using liquid N₂, lyophilized for 24 h, and stored at −80° C. until use.

Preparation of samples for ThT assays. Using cyclic peptide stock solution (100 μM) and PBS, 400 μL aqueous solution of cyclic peptide was prepared in a low-protein-binding Eppendorf tube to achieve the desired cyclic peptide:hIAPP molar ratio (80 μM for 4:1, 40 μM for 2:1, 20 μM for 1:1, 10 μM for 0.5:1, 0 μM for 0:1). 30 μL of the solution was then loaded to wells on a black clear-bottom low-binding 96-well plate (Corning 3881) to make a triplicate for each condition. This plate loaded with aqueous solutions of cyclic peptides was then ready for adding hIAPP.

Preparation and addition of hIAPP for ThT assays. Using ThT stock solution and PBS, 1 mL ThT solution (10 μM) was prepared in a low-protein-binding Eppendorf tube and chilled on ice. 0.5 mL of the pre-chilled ThT solution was added to one aliquot of lyophilized hIAPP, which was equilibrated to room temperature before opening the tube. Pipetting up and down four times was performed to facilitate complete dissolving of hIAPP. The 0.5 mL hIAPP-containing solution was immediately transferred to the remaining 0.5 mL ThT solution on ice. Pipetting up and down four times was performed to facilitate complete mixing. The resulting 1 mL hIAPP solution, where [hIAPP]=20 μM, was immediately transferred to a 25 mL liquid reservoir. Using a multi-channel pipette, 30 μL of the hIAPP solution was quickly loaded into wells containing aqueous solutions of cyclic peptides on the 96-well plate. Final [hIAPP]=10 μM, [ThT]=5 μM.

ThT assay conditions. The plate was then quickly covered and incubated in a Tecan M Plex microplate reader under quiescent condition at 32° C. for at least 12 h. ThT fluorescence at 480 nm was measured every 5 min through the bottom of the plate using an excitation wavelength of 440 nm.

Fitting of aggregation kinetics. ThT aggregation kinetics data was fit to a sigmoidal function using GraphPad Prism and the aggregation lag time (t_{lag}) and the apparent fibril elongation rate (kₐₚₚ) calculated as described.⁵⁵

### Transmission Electron Microscopy

Transmission electron microscopy (TEM) was performed at the University of Wisconsin School of Medicine and Public Health Electron Microscopy facility. hIAPP and cyclic peptide were dissolved in 25 mM sodium phosphate buffer (pH 6.8 with 0.4% DMSO) to achieve a molar ratio of 1:4 at concentrations of hIAPP=10 μM and cyclic peptide=40 μM. The sample was incubated quiescently at 32° C. before TEM analysis. 2 μL of the sample was placed onto Formvar-coated copper grids and the excess liquid was blotted away. 2 μL of diluted Nano-W solution was then added to stain the grids and the excess liquid was blotted away. The TEM analysis was performed on Philips CM120 at 80 kV and digital images were obtained with an AMT BioSprint12 camera.

nESI-MS Analysis

hIAPP was dissolved in 200 mM ammonium acetate buffer (pH 7.4) to prepare 100 μM hIAPP stock solution. Cyclic peptide was first dissolved in DMSO to 10 mM, then diluted into the buffer to make 1 mM cyclic peptide stock solution. These stock solutions were diluted into the buffer to achieve final concentrations of [hIAPP]=16 μM and [cyclic peptide]=64 μM. The percentage of DMSO in the final solution was 0.64% for all the samples. Prepared samples were incubated quiescently for 20 min at 32° C. Native ESI-MS analysis was then performed on a SELECT SERIES Cyclic IMS Q-TOF (Waters Corp., Wilmslow, U.K.) equipped with nano-ESI interface.

All the samples were analyzed using positive ionization ESI with a capillary voltage of 1.1 kV. The following instrumental parameters were used: source temperature 100° C.; desolvation temperature 250° C.; sampling cone 30 V; backing pressure 2.5 mbar; trap collision energy 6 V; trap DC −4 V; transfer collision energy 4 V. The system was calibrated with NaI cluster ions from a 2 μg/μL 50:50 2-propanol:water solution. Data were acquired over the m/z range of 50-8000 and processed using MassLynxV4.2 (Waters Corp., Wilmslow, U.K.).

### Further Optimization of SICLOPPS-Encoding SP Through Evolution on a More Stringent Selection

We subjected the final phage pool from PACE 1 (FIG. 8B) to an additional ˜250 h of PACE using a more stringent AP (pTW357d5) (FIG. 10A). This AP increases selection stringency by installing an inactivating mutation in T7c, thus requiring more copies of folded T7n to be made in order to trigger a level of gill expression sufficient for phage propagation. The stringencies of APs used in this work are ranked as: pTW006ap1a (low), pTW357b (medium), and pTW357d5 (high). Sequencing of four clonal SP from this second PACE revealed that the evolved population fixed several mutations in the intein portion of the SICLOPPS construct, including a truncation of the last 21 amino acids of the Ssp DnaE N-terminal intein (FIG. 10B). Three out of these four SP exhibited markedly increased propagation activity on cells encoding the AP42 aggregation selection compared to the parent SP (FIG. 10C). To determine if the propagation of evolved SP still depended on the activity of the cyclic peptide sequence, we cloned in a negative control sequence (CEAGQLL (SEQ ID NO:39)) where we mutated three positions important for AP42 aggregation inhibition by CKVWQLL³¹ (SEQ ID NO:30) and tested the propagation activity of the resultant phage. 3 out of 4 SP clones showed higher propagation activity when encoding CKVWQLL (SEQ ID NO:30) compared to this negative control sequence, indicating that phage fitness remained sequence-dependent (FIG. 10C). SP clone 1 provided the largest discrimination between CKVWQLL (SEQ ID NO:30) and CEAGQLL (SEQ ID NO:39) and chosen for downstream experiments.

We characterized this more evolved SP through a series of experiments. First, we tested whether phage propagation was splicing-dependent using CKVWQLL SP where the first cysteine in the Ssp N-terminal intein was mutated to alanine, preventing formation of the first splicing intermediate (FIG. 7). As was seen from the SP isolated from PACE 1 and the parent SP, inactivation did not impact propagation of CKVWQLL SP evolved in PACE 2 (FIG. 10C) or its activity on a AP42-GFP folding reporter (FIG. 10D). Thus, we cannot rule out whether the SP-encoded cyclic peptide is being selected for activity as an intein-bound or spliced form, or both.

### Abbreviations for Solid-Phase Peptide Synthesis Methods

Dbz=3,4-diaminobenzoic acid
DCM=dichloromethane
DIPEA=diisopropylethylamine
DMF=dimethylformamide
Fmoc=9-fluorenylmethyloxycarbonyl
HATU=1-[Bis(dimethylamino)methylene]-1H-1,2,3-triazolo[4,5-b]pyridinium 3-oxide hexafluorophosphate
HPLC=high-performance liquid chromatography
MeCN=acetonitrile
Nbz=N-acylbenzimidazolinone
TFA=trifluoroacetic acid
TIPS=triisopropylsilane

SEQUENCES
PROMOTERS
P_{T7a} (attenuated T7 promoter)
(SEQ ID NO: 97)
taatacgtctcactataggg
Pₚᵣₒ₁⁵⁷
(SEQ ID NO: 98)
aacaccacgtcgtccctatctgctgccctaggtctatgagtggttgctggataactttacgggcatgcataaggctcggtatctatattcagggagaccac
aacggtttccctctacaaataattttgttt
Pₚᵣₒ₅⁵⁷
(SEQ ID NO: 99)
aacaccacgtcgtccctatctgctgccctaggtctatgagtggttgctggataactttacgggcatgcataaggctcgtaggatatattcagggagaccac
aacggtttccctctacaaataattttgttt
Pₚₛₚ
(SEQ ID NO: 100)
gatgaaattcgccacttgttagtgtaattcgctaactcatcctggcatgttgctgttgattcttcaatcagatctttataaatcaaaaagataaaaaattg
gcacgcaaattgtattaacagttcagcaggacaatcctgaacgcagaaatcaagaggacaacattacctgc
RIBOSOME BINDING SITES
sd5⁵⁶
(SEQ ID NO: 101)
aaaaaaggaaaaaa
SD8⁵⁶
(SEQ ID NO: 102)
aaggaggaaaaaaaa
GENES
glll (recoded)
(SEQ ID NO: 103)
atgaaaaaattattattcgcaattcctttagttgttcctttctattctcactccgctgaaactgttgaaagttgtttagcaaaaccccatacagaaaattc
atttactaacgtctggaaagacgacaaaactttagatcgttacgctaactatgagggctgtctgtggaatgctacaggcgttgtagtttgtactggtgacg
aaactcagtgttacggtacatgggttcctattgggcttgctatccctgaaaatgagggtggtggctctgagggtggtggttctgagggtggcggttctgag
ggtggcggtactaaacctcctgagtacggtgatacacctattccgggctatacttatatcaaccctctcgacggcacttatccgcctggtactgagcaaaa
ccccgctaatcctaatccttctcttgaggagtctcagcctcttaatactttcatgtttcagaataataggttccgaaataggcagggggcattaactgttt
atacgggcactgttactcaaggcactgaccccgttaaaacttattaccagtacactcctgtatcatcaaaagccatgtatgacgcttactggaacggtaaa
ttcagagactgcgctttccattctggctttaatgaggatccattcgtttgtgaatatcaaggccaatcgtctgacctgcctcaacctcctgtcaatgctgg
cggcggctctggtggtggttctggtggcggctctgagggtggtggctctgagggtggcggttctgagggtggcggctctgagggaggcggttccggtggtg
gctctggttccggtgattttgattatgaaaagatggcaaacgctaataagggggctatgaccgaaaatgccgatgaaaacgcgctacagtctgacgctaaa
ggcaaacttgattctgtcgctactgattacggtgctgctatcgatggtttcattggtgacgtttccggccttgctaatggtaatggtgctactggtgattt
tgctggctctaattcccaaatggctcaagtcggtgacggtgataattcacctttaatgaataatttccgtcaatatttaccttccctccctcaatcggttg
aatgtcgcccttttgtctttggcgctggtaaaccttacgagttcagtatcgactgcgataagatcaacctgttccgcggtgtctttgcgtttcttttatat
gttgccacctttatgtatgtattttctacgtttgctaacatactgcgtaataaggagtcttaa
glll-neg
(SEQ ID NO: 104)
atgaaaaaattattattcgcaattcctttagttgttcctttctattctcactccgctgaaactgttcatcaccatcaccatcacgctgaaactgttgaaag
ttgtttagcaaaaccccatacagaaaattcatttactaacgtctggaaagacgacaaaactttagatcgttacgctaactatgagggctgtctgtggaatg
ctacaggcgttgtagtttgtactggtgacgaaactcagtgttacggtacatgggttcctattgggcttgctatccctgaaaatgagggtggtggctctgag
ggtggcggttctgagggtggcggttctgagggtggcggtactaaacctcctgagtacggtgatacacctattccgggctatacttatatcaaccctctcga
cggcacttatccgcctggtactgagcaaaaccccgctaatcctaatccttctcttgaggagtctcagcctcttaatactttcatgtttcagaataataggt
tccgaaataggcagggggcattaactgtttatacgggcactgttactcaaggcactgaccccgttaaaacttattaccagtacactcctgtatcatcaaaa
gccatgtatgacgcttactggaacggtaaattcagagactgcgctttccattctggctttaatgaggatccattcgtttgtgaatatcaaggccaatcgtc
tgacctgcctcaacctcctgtcaatgctggcggcggctctggtggtggttctggtggcggctctgagggtggtggctctgagggtggcggttctgagggtg
gcggctctgagggaggcggttccggtggtggctcttcccaaatggctcaagtcggtgacggtgataattcacctttaatgaataatttccgtcaatattta
ccttccctccctcaatcggttgaatgtcgcccttttgtctttggcgctggtaaaccttacgagttcagtatcgactgcgataagatcaacctgttccgcgg
tgtctttgcgtttcttttatatgttgccacctttatgtatgtattttctacgtttgctaacatactgcgtaataaggagtcttaa
T7c R632S
(SEQ ID NO: 105)
atgaaagcatttatgcaagttgtcgaggctgacatgctctctaagggtctactcggtggcgaggcgtggtcttcgtggcataaggaagactctattcatgt
aggagtacgctgcatcgagatgctcattgagtcaaccggaatggttagcttacaccgccaaaatgctggcgtagtaggtcaagactctgagactatcgaac
tcgcacctgaatacgctgaggctatcgcaacccgtgcaggtgcgctggctggcatctctccgatgttccaaccttgcgtagttcctcctaagccgtggact
ggcattactggtggtggctattgggctaacggtcgtcgtcctctggcgctggtgcgtactcacagtaagaaagcactgatgcgctacgaagacgtttacat
gcctgaggtgtacaaagcgattaacattgcgcaaaacaccgcatggaaaatcaacaagaaagtcctagcggtcgccaacgtaatcaccaagtggaagcatt
gtccggtcgaggacatccctgcgattgagcgtgaagaactcccgatgaaaccggaagacatcgacatgaatcctgaggctctcaccgcgtggaaacgtgct
gccgctgctgtgtaccgcaaggacaaggctcgcaagtctcgccgtatcagccttgagttcatgcttgagcaagccaataagtttgctaaccataaggccat
ctggttcccttacaacatggactggcgcggtcgtgtttacgctgtgtcaatgttcaacccgcaaggtaacgatatgaccaaaggactgcttacgctggcga
aaggtaaaccaatcggtaaggaaggttactactggctgaaaatccacggtgcaaactgtgcgggtgtcgataaggttccgttccctgagcgcatcaagttc
attgaggaaaaccacgagaacatcatggcttgcgctaagtctccactggagaacacttggtgggctgagcaagattctccgttctgcttccttgcgttctg
ctttgagtacgctggggtacagcaccacggcctgagctataactgctcccttccgctggcgtttgacgggtcttgctctggcatccagcacttctccgcga
tgctccgagatgaggtaggtggtcgcgcggttaacttgcttcctagtgaaaccgttcaggacatctacgggattgttgctaagaaagtcaacgagattcta
caagcagacgcaatcaatgggaccgataacgaagtagttaccgtgaccgatgagaacactggtgaaatctctgagaaagtcaagctgggcactaaggcact
ggctggtcaatggctggcttacggtgttactcgcagtgtgactaagagttcagtcatgacgctggcttacgggtccaaagagttcggcttccgtcaacaag
tgctggaagataccattcagccagctattgattccggcaagggtctgatgttcactcagccgaatcaggctgctggatacatggctaagctgatttgggaa
tctgtgagcgtgacggtggtagctgcggttgaagcaatgaactggcttaagtctgctgctaagctgctggctgctgaggtcaaagataagaagactggaga
gattcttcgcaagcgttgcgctgtgcattgggtaactcctgatggtttccctgtgtggcaggaatacaagaagcctattcagacgcgcttgaacctgatgt
tcctcggtcagttccgcttacagcctaccattaacaccaacaaagatagcgagattgatgcacacaaacaggagtctggtatcgctcctaactttgtacac
agccaagacggtagccaccttcgtaagactgtagtgtgggcacacgagaagtacggaatcgaatcttttgcactgattcacgactccttcggtaccattcc
ggctgacgctgcgaacctgttcaaagcagtgcgcgaaactatggttgacacatatgagtcttgtgatgtactggctgatttctacgaccagttcgctgacc
agttgcacgagtctcaattggacaaaatgccagcacttccggctaaaggtaacttgaacctccgtgacatcttagagtcggacttcgcgttcgcgtaa
T7c R632S/Q649S
(SEQ ID NO: 106)
atgaaagcatttatgcaagttgtcgaggctgacatgctctctaagggtctactcggtggcgaggcgtggtcttcgtggcataaggaagactctattcatgt
aggagtacgctgcatcgagatgctcattgagtcaaccggaatggttagcttacaccgccaaaatgctggcgtagtaggtcaagactctgagactatcgaac
tcgcacctgaatacgctgaggctatcgcaacccgtgcaggtgcgctggctggcatctctccgatgttccaaccttgcgtagttcctcctaagccgtggact
ggcattactggtggtggctattgggctaacggtcgtcgtcctctggcgctggtgcgtactcacagtaagaaagcactgatgcgctacgaagacgtttacat
gcctgaggtgtacaaagcgattaacattgcgcaaaacaccgcatggaaaatcaacaagaaagtcctagcggtcgccaacgtaatcaccaagtggaagcatt
gtccggtcgaggacatccctgcgattgagcgtgaagaactcccgatgaaaccggaagacatcgacatgaatcctgaggctctcaccgcgtggaaacgtgct
gccgctgctgtgtaccgcaaggacaaggctcgcaagtctcgccgtatcagccttgagttcatgcttgagcaagccaataagtttgctaaccataaggccat
ctggttcccttacaacatggactggcgcggtcgtgtttacgctgtgtcaatgttcaacccgcaaggtaacgatatgaccaaaggactgcttacgctggcga
aaggtaaaccaatcggtaaggaaggttactactggctgaaaatccacggtgcaaactgtgcgggtgtcgataaggttccgttccctgagcgcatcaagttc
attgaggaaaaccacgagaacatcatggcttgcgctaagtctccactggagaacacttggtgggctgagcaagattctccgttctgcttccttgcgttctg
ctttgagtacgctggggtacagcaccacggcctgagctataactgctcccttccgctggcgtttgacgggtcttgctctggcatccagcacttctccgcga
tgctccgagatgaggtaggtggtcgcgcggttaacttgcttcctagtgaaaccgttcaggacatctacgggattgttgctaagaaagtcaacgagattcta
caagcagacgcaatcaatgggaccgataacgaagtagttaccgtgaccgatgagaacactggtgaaatctctgagaaagtcaagctgggcactaaggcact
ggctggtcaatggctggcttacggtgttactcgcagtgtgactaagagttcagtcatgacgctggcttacgggtccaaagagttcggcttccgtcaaagtg
tgctggaagataccattcagccagctattgattccggcaagggtctgatgttcactcagccgaatcaggctgctggatacatggctaagctgatttgggaa
tctgtgagcgtgacggtggtagctgcggttgaagcaatgaactggcttaagtctgctgctaagctgctggctgctgaggtcaaagataagaagactggaga
gattcttcgcaagcgttgcgctgtgcattgggtaactcctgatggtttccctgtgtggcaggaatacaagaagcctattcagacgcgcttgaacctgatgt
tcctcggtcagttccgcttacagcctaccattaacaccaacaaagatagcgagattgatgcacacaaacaggagtctggtatcgctcctaactttgtacac
agccaagacggtagccaccttcgtaagactgtagtgtgggcacacgagaagtacggaatcgaatcttttgcactgattcacgactccttcggtaccattcc
ggctgacgctgcgaacctgttcaaagcagtgcgcgaaactatggttgacacatatgagtcttgtgatgtactggctgatttctacgaccagttcgctgacc
agttgcacgagtctcaattggacaaaatgccagcacttccggctaaaggtaacttgaacctccgtgacatcttagagtcggacttcgcgttcgcgtaa
Aß42-T7n
GGSGG linker in underline
T7n in italics
(SEQ ID NO: 107)
atggacgcagagttccgtcacgacagtggctatgaagtccaccaccagaaattggtttttttcgccgaggatgttggtagcaataaaggggctatcatcgg
tttaatggtaggtggagttgttattgcaggtggatctggtggtaacacgattaacatcgctaagaacgacttctctgacatcgaactggctgctatcccgt
tcaacactctggctgaccattacggtgagcgtttagctcgcgaacagttggcccttgagcatgagtcttacgagatgggtgaagcacgcttccgcaagatg
tttgagcgtcaacttaaagctggtgaggttgcggataacgctgccgccaagcctctcatcactaccctactccctaagatgattgcacgcatcaacgactg
gtttgaggaagtgaaagctaagcgcggcaagcgcccgacagccttccagttcctgcaagaaatcaagccggaagccgtagcgtacatcaccattaagacca
ctctggcttgcctaaccagtgctgacaatacaaccgttcaggctgtagcaagcgcaatcggtcgggccattgaggacgaggctcgcttcggtcgtatccgt
gaccttgaagctaagcacttcaagaaaaacgttgaggaacaactcaacaagcgcgtagggcacgtctacaagtaa
AB42 F19S/L34P-T7n
GGSGG linker in underline
T7n in italics
(SEQ ID NO: 108)
atggacgcagagttccgtcacgacagtggctatgaagtccaccaccagaaattggtttctttcgccgaggatgttggtagcaataaaggggctatcatcgg
tccgatggtaggtggagttgttattgcaggtggatctggtggtaacacgattaacatcgctaagaacgacttctctgacatcgaactggctgctatcccgt
tcaacactctggctgaccattacggtgagcgtttagctcgcgaacagttggcccttgagcatgagtcttacgagatgggtgaagcacgcttccgcaagatg
tttgagcgtcaacttaaagctggtgaggttgcggataacgctgccgccaagcctctcatcactaccctactccctaagatgattgcacgcatcaacgactg
gtttgaggaagtgaaagctaagcgcggcaagcgcccgacagccttccagttcctgcaagaaatcaagccggaagccgtagcgtacatcaccattaagacca
ctctggcttgcctaaccagtgctgacaatacaaccgttcaggctgtagcaagcgcaatcggtcgggccattgaggacgaggctcgcttcggtcgtatccgt
gaccttgaagctaagcacttcaagaaaaacgttgaggaacaactcaacaagcgcgtagggcacgtctacaagtaa
hlAPP-T7n
GGSGG linker in underline
T7n in italics
(SEQ ID NO: 109)
atgaaatgcaacactgccacatgtgcaacgcagcgcctggcaaattttttagttcattccagcaacaactttggtgccattctctcatctaccaacgtggg
atccaatacatatggtggatctggtggtaacacgattaacatcgctaagaacgacttctctgacatcgaactggctgctatcccgttcaacactctggctg
accattacggtgagcgtttagctcgcgaacagttggcccttgagcatgagtcttacgagatgggtgaagcacgcttccgcaagatgtttgagcgtcaactt
aaagctggtgaggttgcggataacgctgccgccaagcctctcatcactaccctactccctaagatgattgcacgcatcaacgactggtttgaggaagtgaa
agctaagcgcggcaagcgcccgacagccttccagttcctgcaagaaatcaagccggaagccgtagcgtacatcaccattaagaccactctggcttgcctaa
ccagtgctgacaatacaaccgttcaggctgtagcaagcgcaatcggtcgggccattgaggacgaggctcgcttcggtcgtatccgtgaccttgaagctaag
cacttcaagaaaaacgttgaggaacaactcaacaagcgcgtagggcacgtctacaagtaa
Parent SICLOPPS precursor for cyclo-CKVWQLL (SEQ ID NO: 30)
intC in bold
peptide sequence in underline
intN in italics
chitin-binding domain in bold underline
(SEQ ID NO: 110)
atggttaaagttatcggtcgtcgttccctcggagtgcaaagaatatttgatattggtcttccccaagaccataattttctgctagccaatggggcgatcgc
ccacaactgtaaggtgtggcagttgttgtgtctcagtttcggcaccgaaattttaaccgttgagtacggcccattgcccattggcaaaattgtgagtgaag
aaattaattgttctgtgtacagtgttgatccagaagggagagtttacacccaggcgatcgcccaatggcatgaccggggagagcaggaagtattggaatat
gaattggaagatggttcagtaatccgagctacctctgaccaccgctttttaaccaccgattatcaactgttggcgatcgaagaaatttttgctaggcaact
ggacttgttgactttagaaaatattaagcaaactgaagaagctcttgacaaccatcgtcttccctttccattacttgacgctgggacaattaaaacgacaa
atcctggtgtatccgcttggcaggtcaacacagcttatactgcgggacaattggtcacatataacggcaagacgtataaatgtttgcagccccacacctcc
ttggcaggatgggaaccatccaacgttcctgccttgtggcagcttcaatga
PACE 2 clone 1 SICLOPPS precursor for cyclo-CKVWQLL (SEQ ID NO: 30)
intC in bold
peptide sequence in underline
truncated intN in italics; new stop codon in capital letters
chitin-binding domain in bold underline
(SEQ ID NO: 111)
atggttaaagttatcggtcgtcgttccttcggagtgcaaagaatatttgatattggtcttccccaagaccataattttctgctagccaatggggcgatcgc
ccacaactgtaaggtgtggcagttgttgtgtctcagtttcggcaccgaaattttaaccgttgagtacggcccattgtccattggcaaaattgtgagtgaag
aaattaattgttctgtgtacagtgttgatccaaaagggaaagtttacacccaggcgatcgcccaatggcatgaccggggagagcaggaagtattggaatat
gaattggaagatggttcagtaatccgagctacctctgaccgccgctttttaaccaccgattatcgactggtggcgatcgaagaaatttttgcaaggcaact
ggacttgttgactttagaaaaaaatTAAgcaaactgaagaagctcttgacaaccatcgtcttccctttccattacttgacgctgggacaattaaaacgaca
aatcctggtgtatccgcttggcaggtcaacacagcttatactgcgggacaattggtcacatataacggcaagacgtataaatgtttgcagccccacacctc
cttggcaggatgggaaccatccaacgttcctgccttgtggcagcttcaatga
PACE 1 clone 2 SICLOPPS precursor for cyclo-CKVWQLL (SEQ ID NO: 30)
intC in bold
peptide sequence underline
truncated intN in italics; new stop codon in capital letters
chitin-binding domain in bold underline
(SEQ ID NO: 112)
atggttaaagttatcggtcgtcgttccctcggagtgcaaagaatatttgatattggtcttccccaagaccataattttctgctagccaatggggcgatcgc
ccacaactgtaaggtgtggcagttgttgtgtctcagtttcggcaccgaaattttaaccgttgagtacggcccattgcccattggcaaaattgtgagtgaag
aaattaattgttctgtgtacagtgttgatccaaaagggagagtttacacccaggcgatcgcccaatggcatgaccggggagagcaggaagtattggaatat
gaattggaagatggttcagtaatccgagctacctctgaccaccgctttttaaccaccgattatcaactgttggcgatcgaagaaatttttgctaggcaact
ggacttgttgactttagaaaaaaatTAAgcaaactgaagaagctcttgacaaccatcgtcttccctttccattacttgacgctgggacaattaaaacgaca
aatcctggtgtatccgcttggcaggtcaacacagcttatactgcgggacaattggtcacatataacggcaagacgtataaatgtttgcagccccacacctc
cttggcaggatgggaaccatccaacgttcctgccttgtggcagcttcaatga
PACE 2 clone 1 SICLOPPS precursor for cyclo-CKVWQLL (SEQ ID NO: 30)
intC in bold
peptide sequence underline
truncated intN in italics; new stop codon in capital letters
chitin-binding domain in bold underline
(SEQ ID NO: 113)
atggttaaagttatcggtcgtcgttccttcggagtgcaaagaatatttgatattggtcttccccaagaccataattttctgctagccaatggggcgatcgc
ccacaactgtaaggtgtggcagttgttgtgtctcagtttcggcaccgaaattttaaccgttgagtacggcccattgtccattggcaaaattgtgagtgaag
aaattaattgttctgtgtacagtgttgatccaaaagggaaagtttacacccaggcgatcgcccaatggcatgaccggggagagcaggaagtattggaatat
gaattggaagatggttcagtaatccgagctacctctgaccgccgctttttaaccaccgattatcgactggtggcgatcgaagaaatttttgcaaggcaact
ggacttgttgactttagaaaaaaatTAAgcaaactgaagaagctcttgacaaccatcgtcttccctttccattacttgacgctgggacaattaaaacgaca
aatcctggtgtatccgcttggcaggtcaacacagcttatactgcgggacaattggtcacatataacggcaagacgtataaatgtttgcagccccacacctc
cttggcaggatgggaaccatccaacgttcctgccttgtggcagcttcaatga

### REFERENCES

1. F. Chiti and C. M. Dobson, Annu. Rev. Biochem., 2017, 86, 27-68.
2. Y. S. Eisele, C. Monteiro, C. Fearns, S. E. Encalada, R. L. Wiseman, E. T. Powers and J. W. Kelly, Nat. Rev. Drug Discov., 2015, 14, 759-780.
3. E. E. Cawood, T. K. Karamanos, A. J. Wilson and S. E. Radford, Biophys. Chem., 2021, 268, 106505.
4. J. C. Saunders, L. M. Young, R. A. Mahood, M. P. Jackson, C. H. Revill, R. J. Foster, D. A. Smith, A. E. Ashcroft, D. J. Brockwell and S. E. Radford, Nat. Chem. Biol., 2016, 12, 94-101.
5. Z. Serkeny, F. Rocha, A. M. Damas, S. Macedo-Ribeiro and P. M. Martins, Chem. Asian J., 2019, 14, 500-508.
6. Y. Xu, R. Maya-Martinez, N. Guthertz, G. R. Heath, I. W. Manfield, A. L. Breeze, F. Sobott, R. Foster and S. E. Radford, Nat. Commun., 2022, 13, 1040.
7. A. J. Doig, M. P. del Castillo-Frias, O. Berthoumieu, B. Tarus, J. Nasica-Labouze, F. Sterpone, P. H. Nguyen, N. M. Hooper, P. Faller and P. Derreumaux, ACS Chem. Neurosci., 2017, 8, 1435-1437.
8. Q. Nie, X. Du and M. Geng, Acta Pharmacol. Sin., 2011, 32, 545-551.
9. A. Zorzi, K. Deyle and C. Heinis, Curr. Opin. Chem. Biol., 2017, 38, 24-29.
10. H. Zhang and S. Chen, RSC Chem. Biol., 2022, 3, 18-31.
11. M. R. Naylor, A. T. Bockus, M.-J. Blanco and R. S. Lokey, Curr. Opin. Chem. Biol., 2017, 38, 141-147.
12. X. Li, T. W. Craven and P. M. Levine, J. Med. Chem., 2022, 65, 11913-11926.
13. C. Sohrabi, A. Foster and A. Tavassoli, Nat. Rev. Chem., 2020, 4, 90-101.
14. Y. Huang, M. M. Wiedmann and H. Suga, Chem. Rev., 2018, 119, 10360-10391.
15. C. P. Scott, E. Abel-Santos, M. Wall, D. C. Wahnon and S. J. Benkovic, Proc. Natl. Acad. Sci., 1999, 96, 13638-13643.
16. X. Yang, K. R. Lennard, C. He, M. C. Walker, A. T. Ball, C. Doigneaux, A. Tavassoli and W. A. Van Der Donk, Nat. Chem. Biol., 2018, 14, 375-380.
17. A. M. King, D. A. Anderson, E. Glassey, T. H. Segall-Shapiro, Z. Zhang, D. L. Niquille, A. C. Embree, K. Pratt, T. L. Williams, D. B. Gordon and others, Nat. Commun., 2021, 12, 6343.
18. J. A. lannuzzelli and R. Fasan, Chem. Sci., 2020, 11, 6202-6208.
19. J. A. Kritzer, S. Hamamichi, J. M. McCaffery, S. Santagata, T. A. Naumann, K. A. Caldwell, G. A. Caldwell and S. Lindquist, Nat. Chem. Biol., 2009, 5, 655-663.
20. D. C. Delivoria, S. Chia, J. Habchi, M. Perni, I. Matis, N. Papaevgeniou, M. Reczko, N. Chondrogianni, C. M. Dobson, M. Vendruscolo and G. Skretas, Bacterial production and direct functional screening of expanded molecular libraries for discovering inhibitors of protein aggregation, 2019, vol. 5.
21. I. Matis, D. C. Delivoria, B. Mavroidi, N. Papaevgeniou, S. Panoutsou, S. Bellou, K. D. Papavasileiou, Z. I. Linardaki, A. V. Stavropoulou, K. Vekrellis, N. Boukos, F. N. Kolisis, E. S. Gonos, M. Margarity, M. G. Papadopoulos, S. Efthimiopoulos, M. Pelecanou, N. Chondrogianni and G. Skretas, Nat. Biomed. Eng., 2017, 1, 838-852.
22. M. S. Packer and D. R. Liu, Nat. Rev. Genet., 2015, 16, 379-394.
23. K. M. Esvelt, J. C. Carlson and D. R. Liu, Nature, 2011, 472, 499-503.
24. T. B. Roth, B. M. Woolston, G. Stephanopoulos and D. R. Liu, ACS Synth. Biol., 2019, 8, 796-806.
25. A. K. Brödel, A. Jaramillo and M. Isalan, Nat. Commun., 2016, 7, 13858.
26. J. A. Dewey, S.-A. Azizi, V. Lu and B. C. Dickinson, ACS Synth. Biol., 2021, 10, 2096-2110.
27. S. M. Miller, T. Wang and D. R. Liu, Nat. Protoc., 2020, 15, 4101-4127.
28. J. Zinkus-Boltz, C. DeValk and B. C. Dickinson, ACS Chem. Biol., 2019, 14, 2757-2767.
29. H. Cheruvara, V. L. Allen-Baume, N. M. Kad and J. M. Mason, J. Biol. Chem., 2015, 290, 7426-7435.
30. L. L. Lee, H. Ha, Y.-T. Chang and M. P. DeLisa, Protein Sci., 2009, 18, 277-286.
31. D. C. Delivoria, S. Chia, J. Habchi, M. Perni, I. Matis, N. Papaevgeniou, M. Reczko, N. Chondrogianni, C. M. Dobson, M. Vendruscolo and others, Sci. Adv., 2019, 5, eaax5108.
32. I. Matis, D. C. Delivoria, B. Mavroidi, N. Papaevgeniou, S. Panoutsou, S. Bellou, K. D. Papavasileiou, Z. I. Linardaki, A. V Stavropoulou, K. Vekrellis and Others, Nat. Biomed. Eng., 2017, 1, 838-852.
33. T. Wang, A. H. Badran, T. P. Huang and D. R. Liu, Nat. Chem. Biol., 2018, 14, 972-980.
34. C. Wurth, N. K. Guimard and M. H. Hecht, J. Mol. Biol., 2002, 319, 1279-1290.
35. B. P. Hubbard, A. H. Badran, J. A. Zuris, J. P. Guilinger, K. M. Davis, L. Chen, S. Q. Tsai, J. D. Sander, J. K. Joung and D. R. Liu, Nat. Methods, 2015, 12, 939-942.
36. B. W. Thuronyi, L. W. Koblan, J. M. Levy, W.-H. Yeh, C. Zheng, G. A. Newby, C. Wilson, M. Bhaumik, O. Shubina-Oleinik, J. R. Holt and others, Nat. Biotechnol., 2019, 37, 1070-1079.
37. A. Tavassoli, Q. Lu, J. Gam, H. Pan, S. J. Benkovic and S. N. Cohen, ACS Chem. Biol., 2008, 3, 757-764.
38. E. Miranda, I. K. Nordgren, A. L. Male, C. E. Lawrence, F. Hoakwie, F. Cuda, W. Court, K. R. Fox, P. A. Townsend, G. K. Packham, S. A. Eccles and A. Tavassoli, J. Am. Chem. Soc., 2013, 135, 10418-10425.
39. A. H. Badran and D. R. Liu, Nat. Commun., 2015, 6, 8425.
40. D. Milardi, E. Gazit, S. E. Radford, Y. Xu, R. U. Gallardo, A. Caflisch, G. T. Westermark, P. Westermark, C. La Rosa and A. Ramamoorthy, Chem. Rev., 2021, 121, 1845-1893.
41. A. Spanopoulou, L. Heidrich, H.-R. Chen, C. Frost, D. Hrle, E. Malideli, K. Hille, A. Grammatikopoulos, J. Bernhagen, M. Zacharias and others, Angew. Chemie, 2018, 130, 14711-14716.
42. P. Krotee, S. L. Griner, M. R. Sawaya, D. Cascio, J. A. Rodriguez, D. Shi, S. Philipp, K. Murray, L. Saelices, J. Lee and others, J. Biol. Chem., 2018, 293, 2888-2902.
43. A. R. Horswill, S. N. Savinov and S. J. Benkovic, Proc. Natl. Acad. Sci., 2004, 101, 15591-15596.
44. J. C. Carlson, A. H. Badran, D. A. Guggiana-Nilo and D. R. Liu, Nat. Chem. Biol., 2014, 10, 216-222.
45. L. E. Buchanan, E. B. Dunkelberger, H. Q. Tran, P.-N. Cheng, C.-C. Chiu, P. Cao, D. P. Raleigh, J. J. De Pablo, J. S. Nowick and M. T. Zanni, Proc. Natl. Acad. Sci., 2013, 110, 19285-19290.
46. K. Sivanesam, I. Shu, K. N. L. Huggins, M. Tatarek-Nossol, A. Kapurniotu and N. H. Andersen, FEBS Lett., 2016, 590, 2575-2583.
47. Y. Mao, L. Yu, R. Yang, C. Ma, L. Qu and P. de B. Harrington, Eur. J. Pharmacol., 2017, 804, 102-110.
48. C. K. Wang and D. J. Craik, Pept. Sci., 2016, 106, 901-909.
49. N. H. Shah, G. P. Dann, M. Vila-Perello, Z. Liu and T. W. Muir, J. Am. Chem. Soc., 2012, 134, 11338-11341.
50. J. E. Townend and A. Tavassoli, ACS Chem. Biol., 2016, 11, 1624-1630.
51. J. Pu, J. Zinkus-Boltz and B. C. Dickinson, Nat. Chem. Biol., 2017, 13, 432-438.
52. J. H. Hu, S. M. Miller, M. H. Geurts, W. Tang, L. Chen, N. Sun, C. M. Zeina, X. Gao, H. A. Rees, Z. Lin and others, Nature, 2018, 556, 57-63.
53. S. M. Miller, T. Wang, P. B. Randolph, M. Arbab, M. W. Shen, T. P. Huang, Z. Matuszek, G. A. Newby, H. A. Rees and D. R. Liu, Nat. Biotechnol., 2020, 38, 471-481.
54. S. M. Miller, T. Wang and D. R. Liu, Nat. Protoc., 2020, 15, 4101-4127.
55. K. Gade Malmos, L. M. Blancas-Mejia, B. Weber, J. Buchner, M. Ramirez-Alvarado, H. Naiki and D. Otzen, Amyloid, 2017, 24, 1-16.
56. Ringquist, S. et al. Translation initiation in Escherichia coli: sequences within the ribosome-binding site. Mol. Microbiol. 6, 1219-1229 (1992).
57. Davis, J. H., Rubin, A. J. & Sauer, R. T. Design, construction and characterization of a set of insulated bacterial promoters. Nucleic Acids Res. 39, 1131-1141 (2011).
58. Brödel AK, Jaramillo A, Isalan M. Engineering orthogonal dual transcription factors for multi-input synthetic promoters. Nat Commun. 2016 Dec. 16; 7:13858.
59. Segall-Shapiro T H, Meyer A J, Ellington A D, Sontag E D, Voigt C A. A ‘resource allocator’ for transcription based on a highly fragmented T7 RNA polymerase. Mol Syst Biol. 2014 Jul. 30; 10(7):742.
60. Shis D L, Bennett M R. Library of synthetic transcriptional AND gates built with split T7 RNA polymerase mutants. Proc Natl Acad Sci USA. 2013 Mar. 26; 110(13):5028-33.
61. M. G. ladanza, M. P. Jackson, E. W. Hewitt, N. A. Ranson, S. E. Radford, A new era for understanding amyloid structures and disease. Nat. Rev. Mol. Cell Biol. 19, 755-773 (2018).
62. Guthertz N, van der Kant R, Martinez R M, Xu Y, Trinh C H, lorga BI, Rousseau F, Schymkowitz J, Brockwell D J, Radford S E. The effect of mutation on an aggregation-prone protein: An in vivo, in vitro, and in silico analysis. Proc Natl Acad Sci USA. 2022 May 31; 119(22):e2200468119.
63. Giasson B I, Lee V M, Trojanowski J Q. Interactions of amyloidogenic proteins. Neuromolecular Med. 2003; 4(1-2):49-58.
64. Valentine J, Tavassoli A. Genetically Encoded Cyclic Peptide Libraries: From Hit to Lead and Beyond. Methods Enzymol. 2018; 610:117-134.
65. Tavassoli A. SICLOPPS cyclic peptide libraries in drug discovery. Curr Opin Chem Biol. 2017 June; 38:30-35.

1-18. (canceled)

19. A head-to-tail cyclic peptide comprising an amino acid sequence selected from the group consisting of DLGVFRXₙ (SEQ ID NO:1), RCVFSGXₙ (SEQ ID NO:2), HVVGVIXₙ (SEQ ID NO:3), HVHSYLXₙ (SEQ ID NO:4), LNYFHGXₙ (SEQ ID NO:5), YILSIGXₙ (SEQ ID NO:6), CGLYNIXₙ (SEQ ID NO:7), CHSFFRXₙ (SEQ ID NO:8), GIRSLGXₙ (SEQ ID NO:9), ISCHYGXₙ (SEQ ID NO:10), IYFHHHXₙ (SEQ ID NO:11), VSYILLXₙ (SEQ ID NO:12), FNLVVDXₙ (SEQ ID NO:13), FFRGSDXₙ (SEQ ID NO:14), NRLDVSXₙ (SEQ ID NO:15), GLGHGNXₙ (SEQ ID NO:16), RVWQLCXₙ (SEQ ID NO:17), IVWQLCXₙ (SEQ ID NO:18), KVWQLAXₙ (SEQ ID NO:19), RVWCARXₙ (SEQ ID NO:20), RVYQVLXₙ (SEQ ID NO:21), QVWSAAXₙ (SEQ ID NO:22), RVSQVLXₙ (SEQ ID NO:23), KVWGGLXₙ (SEQ ID NO:24), RVYPVLXₙ (SEQ ID NO:25), QVWSARXₙ (SEQ ID NO:26), QVWCARXₙ (SEQ ID NO:27), TVWTCLXₙ (SEQ ID NO:28), and KVYTAPXₙ (SEQ ID NO:29) wherein X is any amino acid and n is an integer from 0-30.

20. The cyclic peptide of claim 19, wherein the cyclic peptide has a sequence selected from the group consisting of DLGVFRXₙ (SEQ ID NO:1), RCVFSGXₙ (SEQ ID NO:2), HVVGVIXₙ (SEQ ID NO:3), HVHSYLXₙ (SEQ ID NO:4), LNYFHGXₙ (SEQ ID NO:5), YILSIGXₙ (SEQ ID NO:6), CGLYNIXₙ (SEQ ID NO:7), CHSFFRXₙ (SEQ ID NO:8), GIRSLGXₙ (SEQ ID NO:9), ISCHYGXₙ (SEQ ID NO:10), IYFHHHXₙ (SEQ ID NO:11), VSYILLXₙ (SEQ ID NO:12), FNLVVDXₙ (SEQ ID NO:13), FFRGSDXₙ (SEQ ID NO:14), NRLDVSXₙ (SEQ ID NO:15), and GLGHGNXₙ (SEQ ID NO:16).

21. The cyclic peptide of claim 19, wherein the cyclic peptide has a sequence selected from the group consisting of DLGVFRXₙ (SEQ ID NO:1), RCVFSGXₙ (SEQ ID NO:2), and HVVGVIXₙ (SEQ ID NO:3).

22. The cyclic peptide of claim 19, wherein the cyclic peptide has a sequence selected from the group consisting of RVWQLCXₙ (SEQ ID NO:17), IVWQLCXₙ (SEQ ID NO:18), and KVWQLAXₙ (SEQ ID NO:19, RVWCARXₙ (SEQ ID NO:20), RVYQVLXₙ (SEQ ID NO:21), QVWSAAXₙ (SEQ ID NO:22), RVSQVLXₙ (SEQ ID NO:23), KVWGGLXₙ (SEQ ID NO:24), RVYPVLXₙ (SEQ ID NO:25), QVWSARXₙ (SEQ ID NO:26), QVWCARXₙ (SEQ ID NO:27), TVWTCLXₙ (SEQ ID NO:28), and KVYTAPXₙ (SEQ ID NO:29).

23. A method of reducing aggregation of an aggregation-prone protein, the method comprising contacting the aggregation-prone protein with a cyclic peptide as recited in claim 19.

24. The method of claim 23, wherein the cyclic Peptide has a sequence selected from the group consisting of DLGVFRXₙ (SEQ ID NO:1), RCVFSGXₙ (SEO ID NO:2), HVVGVIXₙ (SEQ ID NO:3), HVHSYLXₙ (SEQ ID NO:4), LNYFHGXₙ (SEO ID NO:5), YILSIGXₙ (SEO ID NO:6), CGLYNIXₙ (SEO ID NO:7), CHSFFRXₙ (SEO ID NO:8), GIRSLGXₙ(SEO ID NO:9), ISCHYGXₙ (SEO ID NO:10), IYFHHHXₙ (SEO ID NO:11), VSYILLXₙ (SEQ ID NO:12), FNLVVDXₙ (SEO ID NO:13), FFRGSDXₙ (SEO ID NO:14), NRLDVSXₙ (SEQ ID NO:15), and GLGHGNXₙ (SEO ID NO:16).

25. The method of claim 24, wherein the aggregation-prone protein comprises human islet amyloid polypeptide.

26. The method of claim 25, wherein the contacting is performed in a subject with type 2 diabetes.

27. The method of claim 23, wherein the cyclic peptide has a sequence selected from the group consisting of RVWQLCXₙ (SEO ID NO:17), IVWQLCXₙ (SEQ ID NO:18), and KVWQLAXₙ (SEQ ID NO:19, RVWCARXₙ (SEQ ID NO:20), RVYQVLXₙ(SEO ID NO:21), QVWSAAXₙ (SEO ID NO:22), RVSQVLXₙ (SEO ID NO:23), KVWGGLXₙ (SEO ID NO:24), RVYPVLXₙ (SEO ID NO:25), QVWSARXₙ (SEO ID NO:26), QVWCARXₙ (SEQ ID NO:27), TVWTCLXₙ (SEQ ID NO:28), and KVYTAPXₙ (SEQ ID NO:29).

28. The method of claim 27, wherein the aggregation-prone protein comprises amyloid-β42.

29. The method of claim 28, wherein the contacting is performed in a subject with Alzheimer's disease.

30. The method of claim 23, wherein the contacting is performed in vivo.

31. The method of claim 23, wherein the contacting is performed in vitro.
